(19) United States
(12) Patent Application Publication
(10) Pub. No.: US 2026/0138949 A1
(43) Pub. Date: May 21, 2026

(54) NOVEL COMPOUND AS PHOSPHOLIPASE D INHIBITOR AND USE THEREOF

(71) Applicant: UIF (UNIVERSITY INDUSTRY FOUNDATION), YONSEI UNIVERSITY, Seoul (KR)

(72) Inventors: Gyoonhee Han, Seoul (KR); Do Sik Min, Seoul (KR); Chang Mok Oh, Seoul (KR); Chulho Lee, Seoul (KR); Doona Song, Seoul (KR); Won Chan Hwang, Seoul (KR); Hye Sung Lee, Seoul (KR)

(73) Assignee: UIF (UNIVERSITY INDUSTRY FOUNDATION), YONSEI UNIVERSITY, Seoul (KR)

(21) Appl. No.: 18/992,341

(22) PCT Filed: Jul. 6, 2023
(86) PCT No.: PCT/KR2023/009613

§ 371 Date: Jan. 8, 2025

(30) Foreign Application Priority Data
Jul. 8, 2022 (KR) 1020220084614

(51) Int. Cl. C07D 209/42; A61K 31/404; A61K 31/405; A61K 31/4188; A61K 31/4439; A61P 35/00; C07D 209/18; C07D 401/12; C07D 495/04

(52) U.S. Cl. CPC C07D 209/42; A61K 31/404; A61K 31/405; A61K 31/4188; A61K 31/4439; A61P 35/00; C07D 209/18; C07D 401/12; C07D 495/04

(57) ABSTRACT

The present invention relates to novel compounds as phospholipase D (PLD) inhibitors and use thereof, more specifically to novel compounds that exhibit excellent phospholipase D inhibitory activity and can be utilized as therapeutic agents for cancer and degenerative neurological diseases.

### TECHNICAL FIELD

The present invention relates to novel compounds functioning as phospholipase D (PLD) inhibitor and use thereof, and more specifically, to novel compounds that exhibit excellent phospholipase D inhibitory activity, which can be utilized as therapeutic agents for cancer and degenerative neurological diseases.

### BACKGROUND OF THE INVENTION

Phospholipase D (PLD) enzymes are phosphodiesterases that play a crucial role in various signaling and metabolic pathways. They are encoded by a gene superfamily1 and can be defined by several highly conserved motifs. These enzymes catalyze the removal of the head group from glycerophospholipids to produce phosphatidic acid (PtdOH), a reaction that results in the stoichiometric release of free head groups1,2,3,4,5,6,7. One of the four subgroups of PLD enzymes is characterized by a conserved H-X-K-X4-D-X6-G-(G/S) catalytic motif, commonly known as the HKD motif Members of this subgroup typically hydrolyze phosphodiester bonds through the HKD catalytic motif using a similar reaction mechanism. However, some family members exhibit lipid hydrolase activity, while others do not. Additionally, several PLD enzymes lacking the HKD motif that produces PtdOH5 have been identified.

In mammalian cells, the isoenzymes PLD1 and PLD2, which contain the HKD motif and highly conserved phox and pleckstrin homology (PX-PH) domains, are ubiquitously present. These two isoenzymes often act as nodes at points where signaling pathways converge. They are known to participate in cellular process involving membrane remodeling or biogenesis, such as vesicle transport, endocytosis, degranulation, and cell cycle progression. While the primary substrate for PLD1 and PLD2 is generally phosphatidylcholine, these enzymes can also hydrolyze other amine-containing glycerophospholipids, including phosphatidylethanolamine, phosphatidylserine, and phosphatidylglycerol.

Direct and indirect inhibitors of PLD activity have been identified despite long-standing skepticism about the efficacy of this pathway. After raloxifene and halopemide were identified as direct inhibitors, isoenzyme-specific compounds were systematically developed to further differentiate the functions of PLD1 and PLD2.

Meanwhile, PLD activity and expression have been found to be upregulated in various types of human cancers, where PLD enzymes function downstream of several known oncogenes. Inhibition of PtdOH production has been shown to significantly impacts tumor formation and malignant invasion.

Additionally, PLD1, PLD2, and PLD3 are implicated in Alzheimer's disease and other neurodegenerative conditions, although the mechanisms explaining the roles of these proteins in central nervous system pathophysiology have not yet been identified.

Therefore, the inhibition of PLD is being increasingly recognized as a therapeutic strategy for cancer and neurodegenerative diseases.

### SUMMARY OF INVENTION

### Problem to be Solved

The present inventors have conducted extensive research to develop novel compounds exhibiting PLD inhibitory activity. As a result, they have discovered a series of compounds represented by Chemical Formula 1, which exhibit excellent PLD inhibitory activity and have preventive or therapeutic effects on cancer and/or degenerative neurological diseases, thereby completing the present invention.

Therefore, an object of the present invention is to provide a compound represented by the following Chemical Formula 1 or a pharmaceutically acceptable salt thereof:

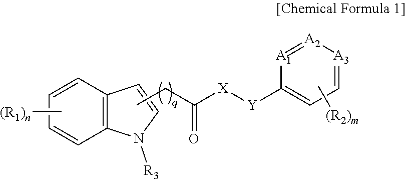

wherein,
R1 is each independently hydrogen, substituted or unsubstituted C1-C10 alkyl, halogen, C1-C10 alkoxycarbonyl, C1-C10 hydroxycarbonyl, or

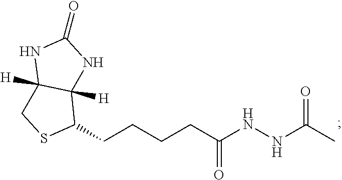

R2 is each independently hydrogen, nitro, halogen, C1-C10 alkoxy, hydroxy, C1-C10 alkoxycarbonyl, substituted or unsubstituted C1-C10 alkylene, or substituted or unsubstituted C1-C6 alkynyl;
R3 is substituted or unsubstituted C1-C10 alkyl;
X is N or;
Y is absent, substituted or unsubstituted C1-C10 alkylene or N;
A1, A2 and A3 are each independently C or N;
n is 1, 2 or 3;
m is 1, 2 or 3;
q is 0, 1, 2, 3, 4 or 5; and
when R1, R2, R3 and Y are substituted, they are each independently substituted with one or more substituents selected from the group consisting of halogen, C1-C10 alkyl, nitro, hydroxy, cyano, amino, thiol, carboxyl, amide, nitrile, sulfide, disulfide, sulfonyl, formyl, formyloxy and formylamino.

Another object of the present invention is to provide a pharmaceutical composition for preventing or treating cancer or degenerative neurological diseases, comprising the compound or a pharmaceutically acceptable salt thereof as an active ingredient.

Additionally, another object of the present invention is to provide a pharmaceutical composition for preventing or treating cancer or degenerative neurological diseases, consisting of the compound or a pharmaceutically acceptable salt thereof.

Furthermore, another object of the present invention is to provide a pharmaceutical composition for preventing or treating cancer or degenerative neurological diseases, consisting essentially of the compound or a pharmaceutically acceptable salt thereof.

Another object of the present invention is to provide the use of the compound or a pharmaceutically acceptable salt thereof for preparing a pharmaceutical composition for treating cancer or a degenerative neurological disease.

Another object of the present invention is to provide a method for treating cancer or a degenerative neurological disease comprising administering an effective amount of a pharmaceutical composition comprising the compound or a pharmaceutically acceptable salt thereof as an active ingredient to a subject in need thereof.

### Means for Solving the Problem

To achieve the aforementioned objectives of the present invention, the present invention provides a compound represented by the following Chemical Formula 1 or a pharmaceutically acceptable salt thereof.

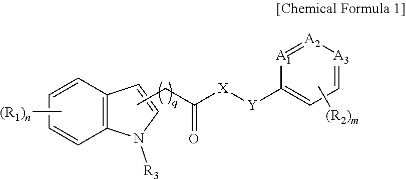

wherein,
R1 is each independently hydrogen, substituted or unsubstituted C1-C10 alkyl, halogen, C1-C10 alkoxycarbonyl, C1-C10 hydroxycarbonyl, or

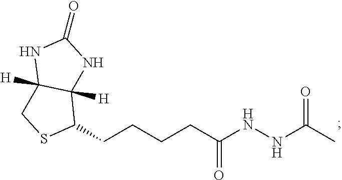

R2 is each independently hydrogen, nitro, halogen, C1-C10 alkoxy, hydroxy, C1-C10 alkoxycarbonyl, substituted or unsubstituted C1-C10 alkylene, or substituted or unsubstituted C1-C6 alkynyl;
R3 is substituted or unsubstituted C1-C10 alkyl;
X is N or O;
Y is absent, substituted or unsubstituted C1-C10 alkylene or N;
A1, A2 and A3 are each independently C or N;
n is 1, 2 or 3;
m is 1, 2 or 3;
q is 0, 1, 2, 3, 4 or 5; and
when R1, R2, R3 and Y are substituted, they are each independently substituted with one or more substituents selected from the group consisting of halogen, C1-C10 alkyl, nitro, hydroxy, cyano, amino, thiol, carboxyl, amide, nitrile, sulfide, disulfide, sulfonyl, formyl, formyloxy and formylamino.

To achieve another objective of the present invention, the present invention provides a pharmaceutical composition for preventing or treating cancer or degenerative neurological diseases, comprising the compound or a pharmaceutically acceptable salt thereof as an active ingredient.

Additionally, to achieve another objective of the present invention, the present invention provides a pharmaceutical composition for preventing or treating cancer or degenerative neurological diseases, consisting of the compound or a pharmaceutically acceptable salt thereof.

Furthermore, to achieve another objective of the present invention, the present invention provides a pharmaceutical composition for preventing or treating cancer or degenerative neurological diseases, consisting essentially of the compound or a pharmaceutically acceptable salt thereof.

To achieve another objective of the present invention, the present invention provides the use of the compound or a pharmaceutically acceptable salt thereof for preparing a pharmaceutical composition for treating cancer or a degenerative neurological disease.

To achieve another objective of the present invention, the present invention provides a method for treating cancer or a degenerative neurological disease comprising administering an effective amount of a pharmaceutical composition comprising the compound or a pharmaceutically acceptable salt thereof as an active ingredient to a subject in need thereof.

Hereinafter, the present invention will be described in detail.

The present invention provides a compound represented by the following Chemical Formula 1 or a pharmaceutically acceptable salt thereof:

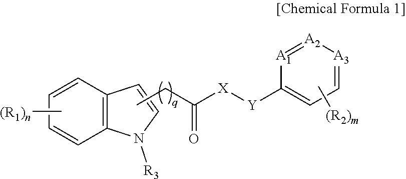

wherein,
R1 is each independently hydrogen, substituted or unsubstituted C1-C10 alkyl, halogen, C1-C10 alkoxycarbonyl, C1-C10 hydroxycarbonyl, or

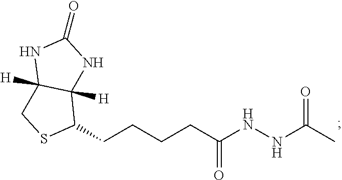

R2 is each independently hydrogen, nitro, halogen, C1-C10 alkoxy, hydroxy, C1-C10 alkoxycarbonyl, substituted or unsubstituted C1-C10 alkylene, or substituted or unsubstituted C1-C6 alkynyl;
R3 is substituted or unsubstituted C1-C10 alkyl;
X is N or;
Y is absent, substituted or unsubstituted C1-C10 alkylene or N;
A1, A2 and A3 are each independently C or N;
n is 1, 2 or 3;
m is 1, 2 or 3;
q is 0, 1, 2, 3, 4 or 5; and
when R1, R2, R3 and Y are substituted, they are each independently substituted with one or more substituents selected from the group consisting of halogen, C1-C10 alkyl, nitro, hydroxy, cyano, amino, thiol, carboxyl, amide, nitrile, sulfide, disulfide, sulfonyl, formyl, formyloxy and formylamino.

In the present invention, ‘halogen’ refers to fluorine (F), chlorine (Cl), bromine (Br), or iodine (I). In the present invention, ‘alkyl’ refers to a monovalent group formed by losing one hydrogen atom from an aliphatic saturated hydrocarbon. In the present invention, alkyl preferably refers to a linear (straight-chain form) or branched (including side-chain form) alkyl type of 1 to 10 carbon atoms (C1, C2, C3, C4, C5, C6, C7, C8, C9, and C10), whether substituted or unsubstituted. Linear or branched alkyls of C1-C10 include, but are not limited to, methyl, ethyl, n-propyl, isopropyl, n-butyl, sec-butyl, tert-butyl, n-pentyl, isopentyl, neopentyl, tert-pentyl, n-hexyl, isohexyl, etc. Preferably, in the present invention, alkyl may be a linear or branched alkyl of C1-C6, whether substituted or unsubstituted, and more preferably, a linear or branched alkyl of C1-C4, whether substituted or unsubstituted.

In the present invention, ‘alkynyl’ refers to an aliphatic unsaturated hydrocarbon group having at least one (i.e., one or more) triple bonds. In the present invention, alkynyl preferably refers to a linear or branched alkynyl type of 1 to 6 carbon atoms (C1, C2, C3, C4, C5, or C6), whether substituted or unsubstituted. Linear or branched alkynyls of C1-C6 include, but are not limited to, ethynyl (—C≡CH), —C≡CH(CH₃), —C≡C(CH₂CH₃), —CH₂C≡CH, —CH₂C≡C(CH₃), and —CH₂C≡C(CH₂CH₃). More preferably, in the present invention, alkynyl may be a linear or branched alkynyl of C1-C4, whether substituted or unsubstituted, and more preferably, a linear or branched alkynyl of C1-C3, whether substituted or unsubstituted.

In the present invention, ‘alkoxy’ refers to ‘—O-alkyl’, and alkyl is as disclosed above. In the present invention, alkoxy preferably refers to a linear or branched alkoxy substituent type of 1 to 10 carbon atoms (C1, C2, C3, C4, C5, C6, C7, C8, C9, and C10), whether substituted or unsubstituted. Linear or branched alkoxys of C1-C10 include, but are not limited to, methoxy, trifluoromethoxy, ethoxy, n-propoxy, isopropoxy, n-butoxy, t-butoxy, sec-butoxy, n-pentoxy, etc. More preferably, in the present invention, alkoxy may be a linear or branched alkoxy of C1-C6, whether substituted or unsubstituted, and more preferably, a linear or branched alkoxy of C1-C4, whether substituted or unsubstituted.

In the present invention, ‘alkylene’ refers to a linear or branched saturated divalent hydrocarbon radical. The alkylene may have 1 to 10 carbon atoms. Preferably, the alkylene may have 2 to 5 carbon atoms. Examples of such alkylene radicals include methylene, ethylene, propylene, etc., but are not limited thereto.

In the present invention, the term ‘substituted’ refers to, unless otherwise specified, including at least one substituent, for example, one or more selected from the group consisting of halogen, C1-C10 alkyl, nitro, hydroxy, cyano, amino, thiol, carboxyl, amide, nitrile, sulfide, disulfide, sulfonyl, formyl, formyloxy, and formylamino. Unless otherwise specified, any group or structure described for the compound represented by Chemical Formula 1 of the present invention may be substituted, provided that the structure obtained by such substitution does not significantly adversely affect the properties of the compound represented by Chemical Formula 1 of the present invention (particularly, the activity related to the intended use of the present invention).

In a preferred embodiment of the present invention, R1 may each independently be hydrogen, halogen, methoxycarbonyl, hydroxycarbonyl, —CF₃ or

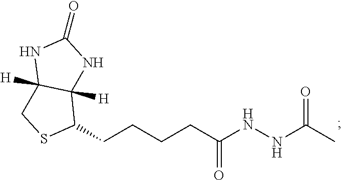

In a preferred embodiment of the present invention, R2 may each independently be hydrogen, nitro, halogen, methoxy, hydroxy, methoxycarbonyl, —CF₃, t-butyl, ethoxycarbonyl, ethynyl, 2-propyl, 2-butyl, or methyl.

In a preferred embodiment of the present invention, R3 may be methyl.

In a preferred embodiment of the present invention, one of A1, A2, and A3 may be N, and the other two may be C.

In a preferred embodiment of the present invention, the compound of Chemical Formula 1 may be characterized by being selected from the group consisting of the following compounds:
N-(4-chloro-3-nitrophenyl)-1H-indole-3-carboxamide,
N-(4-bromophenyl)-1H-indole-3-carboxamide,
N-(3,4-dimethoxyphenyl)-1H-indole-3-carboxamide,
N-(3,4-dichlorophenyl)-1H-indole-3-carboxamide,
N-(3,4-dichlorobenzyl)-1H-indole-3-carboxamide,
N′-(3,4-dichlorophenyl)-1H-indole-2-carbohydrazide,
N-(2-hydroxyphenyl)-2-(1H-indol-3-yl)acetamide,
N-(3-hydroxyphenyl)-2-(1H-indol-3-yl)acetamide,
2-(1H-indol-3-yl)-N-(2-methoxyphenyl)acetamide,
2-(1H-indol-3-yl)-N-(3-methoxyphenyl)acetamide,
2-(1H-indol-3-yl)-N-(4-methoxyphenyl)acetamide,
N-(2-fluorophenyl)-2-(1H-indol-3-yl)acetamide,
N-(3-fluorophenyl)-2-(1H-indol-3-yl)acetamide,
N-(4-fluorophenyl)-2-(1H-indol-3-yl)acetamide,
N-(2-bromophenyl)-2-(1H-indol-3-yl)acetamide,
N-(3-bromophenyl)-2-(1H-indol-3-yl)acetamide,
N-(4-bromophenyl)-2-(1H-indol-3-yl)acetamide,
N-(3,5-difluorophenyl)-2-(1H-indol-3-yl)acetamide,
N-(4-chloro-3-nitrophenyl)-2-(1H-indol-3-yl)acetamide,
N-(3,4-dichlorophenyl)-2-(1H-indol-3-yl)acetamide,
N-(5-bromopyridin-2-yl)-2-(1H-indol-3-yl)acetamide,
N-(5-fluoropyridin-2-yl)-2-(1H-indol-3-yl)acetamide,
2-(1H-indol-3-yl)-N-phenylacetamide,
N-(4-hydroxyphenyl)-2-(1H-indol-3-yl)acetamide,
methyl 4-(2-(1H-indol-3-yl)acetamido)benzoate,
2-(1H-indol-3-yl)-N-(4-(trifluoromethyl)phenyl)acetamide,
N-(4-tert-butylphenyl)-2-(1H-indol-3-yl)acetamide,
N-(3,4-dimethoxyphenyl)-2-(1H-indol-3-yl)acetamide,
ethyl 4-(2-(1H-indol-3-yl)acetamido)benzoate,
2-(1H-indol-3-yl)-N-(3-nitrophenyl)acetamide,
N-(4-ethynylphenyl)-2-(1H-indol-3-yl)acetamide,
N-(3,5-bis(trifluoromethyl)phenyl)-2-(1H-indol-3-yl)acetamide,
2-(1H-indol-3-yl)-N-(4-isopropylphenyl)acetamide,
N-(4-sec-butylphenyl)-2-(1H-indol-3-yl)acetamide,
N-(2-fluorobenzyl)-2-(1H-indol-3-yl)acetamide,
N-(3-fluorobenzyl)-2-(1H-indol-3-yl)acetamide,
N-(4-fluorobenzyl)-2-(1H-indol-3-yl)acetamide,
methyl 4-((2-(1H-indol-3-yl)acetamido)methyl)benzoate,
2-(1H-indol-3-yl)-N-(4-methoxybenzyl)acetamide,
2-(1H-indol-3-yl)-N-(3-methylbenzyl)acetamide,
N-(4-tert-butylbenzyl)-2-(1H-indol-3-yl)acetamide,
2-(1H-indol-3-yl)-N-(3-(trifluoromethyl)benzyl)acetamide,
2-(1H-indol-3-yl)-N-(4-(trifluoromethyl)benzyl)acetamide,
N-(2,4-dimethoxybenzyl)-2-(1H-indol-3-yl)acetamide,
N-(3,4-dimethoxybenzyl)-2-(1H-indol-3-yl)acetamide,
2-(1H-indol-3-yl)-N-(4-nitrobenzyl)acetamide,
N-(2,3-dichlorobenzyl)-2-(1H-indol-3-yl)acetamide,
N-(2-chlorobenzyl)-2-(1H-indol-3-yl)acetamide,
N-(4-chlorobenzyl)-2-(1H-indol-3-yl)acetamide,
2-(1H-indol-3-yl)-N-(2-methoxybenzyl)acetamide,
N′-(2-chlorophenyl)-2-(1H-indol-3-yl)acetohydrazide,
N′-(3-chlorophenyl)-2-(1H-indol-3-yl)acetohydrazide,
N′-(4-chlorophenyl)-2-(1H-indol-3-yl)acetohydrazide,
N′-(3,4-dichlorophenyl)-2-(1H-indol-3-yl)acetohydrazide,
N′-(4-bromophenyl)-2-(1H-indol-3-yl)acetohydrazide,
2-(1H-indol-3-yl)-N-(pyridin-2-ylmethyl)acetamide,
2-(1H-indol-3-yl)-N-(pyridin-3-ylmethyl)acetamide,
2-(1H-indol-3-yl)-N-(pyridin-4-ylmethyl)acetamide,
2-(1H-indol-3-yl)-N-phenethylacetamide,
N-(4-bromophenethyl)-2-(1H-indol-3-yl)acetamide,
N-(2-fluorophenyl)-3-(1H-indol-3-yl)propanamide,
N-(3-fluorophenyl)-3-(1H-indol-3-yl)propanamide,
N-(4-fluorophenyl)-3-(1H-indol-3-yl)propanamide,
N-(3-bromophenyl)-3-(1H-indol-3-yl)propanamide,
N-(4-bromophenyl)-3-(1H-indol-3-yl)propanamide,
N-(3,5-difluorophenyl)-3-(1H-indol-3-yl)propanamide,
N-(3,5-bis(trifluoromethyl)phenyl)-3-(1H-indol-3-yl)propanamide,
N-(4-chloro-3-nitrophenyl)-3-(1H-indol-3-yl)propanamide,
N-(3,4-dichlorophenyl)-3-(1H-indol-3-yl)propanamide,
N-(3,4-dimethoxyphenyl)-3-(1H-indol-3-yl)propanamide,
3-(1H-indol-3-yl)-N-(4-methoxybenzyl)propanamide,
N-(3,4-dichlorobenzyl)-3-(1H-indol-3-yl)propanamide,
N-(3,4-dimethoxybenzyl)-3-(1H-indol-3-yl)propanamide,
N-(3-fluorophenyl)-4-(1H-indol-3-yl)butanamide,
N-(4-fluorophenyl)-4-(1H-indol-3-yl)butanamide,
N-(4-bromophenyl)-4-(1H-indol-3-yl)butanamide,
N-(3,5-bis(trifluoromethyl)phenyl)-4-(1H-indol-3-yl)butanamide,
N-(4-chloro-3-nitrophenyl)-4-(1H-indol-3-yl)butanamide,
N-(3,4-dichlorophenyl)-4-(1H-indol-3-yl)butanamide,
methyl 4-((4-(1H-indol-3-yl)butanamido)methyl)benzoate,
4-(1H-indol-3-yl)-N-(4-methoxybenzyl)butanamide,
N-(3,4-dichlorobenzyl)-4-(1H-indol-3-yl)butanamide,
N-(4-fluorophenyl)-1H-indole-2-carboxamide,
N-(3,5-bis(trifluoromethyl)phenyl)-1H-indole-2-carboxamide,
N-(4-chloro-3-nitrophenyl)-1H-indole-2-carboxamide,
N-(3,4-dichlorophenyl)-1H-indole-2-carboxamide,
N-(3,4-dimethoxyphenyl)-1H-indole-2-carboxamide,
methyl 4-((1H-indole-2-carboxamido)methyl)benzoate,
N′-(3,4-dichlorophenyl)-1H-indole-2-carbohydrazide,
5-fluoro-N-(4-fluorophenyl)-1H-indole-2-carboxamide,
N-(3,5-bis(trifluoromethyl)phenyl)-5-fluoro-1H-indole-2-carboxamide,
N-(3,5-difluorophenyl)-5-fluoro-1H-indole-2-carboxamide,
N-(3,4-dimethoxyphenyl)-5-fluoro-1H-indole-2-carboxamide,
N-(3,4-dimethoxybenzyl)-5-fluoro-1H-indole-2-carboxamide,
5-fluoro-N-(3-(trifluoromethyl)phenyl)-1H-indole-2-carboxamide,
N-(3,5-dimethoxyphenyl)-5-fluoro-1H-indole-2-carboxamide,
5-fluoro-N-(2-fluorophenyl)-1H-indole-2-carboxamide,
5-fluoro-N-(3-fluorophenyl)-1H-indole-2-carboxamide,
N-(3,5-bis(trifluoromethyl)phenyl)-5-chloro-1H-indole-2-carboxamide,
methyl 2-((3,5-bis(trifluoromethyl)phenyl)carbamoyl)-1H-indole-5-carboxylate,
2-((3,5-bis(trifluoromethyl)phenyl)carbamoyl)-1H-indole-5-carboxylic acid,
N-(3,5-bis(trifluoromethyl)phenyl)-5-(trifluoromethyl)-1H-indole-2-carboxamide,
N-(3,5-dimethylphenyl)-5-fluoro-1H-indole-2-carboxamide,
N-(3,5-dimethylphenyl)-1H-indole-2-carboxamide,
N-(3,4-dihydroxyphenyl)-1H-indole-3-carboxamide,
N-(3,4-dihydroxyphenyl)-2-(1H-indol-3-yl)acetamide,
N-(3,4-dihydroxybenzyl)-2-(1H-indol-3-yl)acetamide,
N-(3,4-dihydroxyphenyl)-3-(1H-indol-3-yl)propanamide,
N-(3,4-dihydroxyphenyl)-1H-indole-2-carboxamide,
N-(3,4-dihydroxyphenyl)-5-fluoro-1H-indole-2-carboxamide,
N-(3,4-dihydroxybenzyl)-5-fluoro-1H-indole-2-carboxamide,
N-(3,5-dihydroxyphenyl)-5-fluoro-1H-indole-2-carboxamide,
N-(3,5-bis(trifluoromethyl)phenyl)-5,6-difluoro-1H-indole-2-carboxamide,
N-(3,5-bis(trifluoromethyl)phenyl)-5,6-difluoro-1-methyl-1H-indole-2-carboxamide,
4-nitrobenzyl 2-(1H-indol-3-yl)acetate,
4-methoxybenzyl 1H-indole-2-carboxylate,
4-methoxybenzyl 4-(1H-indol-3-yl)butanoate,
3,5-bis(trifluoromethyl)phenyl 5-fluoro-1H-indole-2-carboxylate or
N-(3,5-bis(trifluoromethyl)phenyl)-5-(2-(5-((3aS,4S,6aR)-2-oxohexahydro-TH-thieno[3,4-d]imidazol-4-yl)pentanoyl)hydrazine-1-carbonyl)-1H-indole-2-carboxamide.

In one embodiment of the present invention, it can be understood that some of the compounds of Chemical Formula 1 provided by the present invention contain one or more chiral centers and thus exist in the form of two or more stereoisomers. Racemates of these isomers, individual isomers enriched in one enantiomer, mixtures, partial stereoisomers with two chiral centers, and mixtures partially enriched in specific stereoisomers are included within the scope of the present invention. A person skilled in the art will understand that the present invention includes individual stereoisomers (e.g., enantiomers), racemates, or partially resolved mixtures of the compounds of Chemical Formula 1, and, as appropriate, individual tautomers.

In one embodiment of the present invention, the present invention provides compounds comprising various purities of stereoisomers, i.e., partial stereoisomers or enantiomers with various “ee” or “de”. In some embodiments, the compound of Chemical Formula I has an enantiomeric purity of at least 60% ee (e.g., 60%, 65%, 70%, 75%, 80%, 85%, 90%, 91%, 92%, 93%, 94%, 95%, 96%, 97%, 98%, 99%, 99.5%, 99.9% ee, or a range between these values). In one embodiment, the compound of Chemical Formula 1 has an enantiomeric purity exceeding 95% ee up to 99.9% ee. In one embodiment, the compound of Chemical Formula 1 (e.g., as described herein) has a partial stereoisomeric purity of at least 60% de (e.g., 60%, 65%, 70%, 75%, 80%, 85%, 90%, 91%, 92%, 93%, 94%, 95%, 96%, 97%, 98%, 99%, 99.5%, 99.9% de, or a range between these values). In one embodiment, the compound of Chemical Formula 1 (e.g., as described herein) has a partial stereoisomeric purity exceeding 99.9% de.

In the present invention, the term “enantiomeric excess” or “ee” refers to the extent to which one enantiomer is present compared to the other components. In a mixture of R and S enantiomers, it is defined as a percentage of enantiomeric excess, where R and S represent the molar or weight ratio of each enantiomer in the mixture, and R+S=1. According to the knowledge of optical rotation of chiral substances, the percentage of enantiomeric excess is defined as ([a]obs/[a]max)*100, where [a]obs is the optical rotation of the enantiomeric mixture, and [a]max is the optical rotation of the pure enantiomer.

In the present invention, the term “diastereomeric excess” or “de” refers to the extent to which one diastereomer is present compared to the other components and is defined similarly to the enantiomeric excess above. Thus, in a mixture of diastereomers D1 and D2, the percentage of diastereomeric excess is defined as (D1−D2)*100, where D1 and D2 represent the molar or weight ratio of each diastereomer in the mixture, and D1+D2=1.

In one embodiment of the present invention, racemates can be used as they are or decomposed into individual isomers. Upon decomposition, a stereochemically pure compound or a mixture enriched in one or more isomers can be provided. Isomer separation methods are known in the art and include physical methods such as chromatography using chiral adsorbents. Each isomer can be prepared in chiral form from chiral precursors. Alternatively, diastereomeric salts can be formed with individual enantiomers such as 10-camphorsulfonic acid, camphoric acid, alpha-bromocamphoric acid, tartaric acid, diacetyl tartaric acid, malic acid, pyrrolidone-5-carboxylic acid, and the like, and these salts can be fractionally crystallized, and one or both of the decomposed bases can be liberated, and optionally, the above process can be repeated to chemically separate individual isomers from the mixture, thereby obtaining the desired stereoisomer in a form with substantial optical purity, i.e., for example, at least 91%, 92%, 93%, 94%, 95%, 96%, 97%, 98%, 99% or 99.5%. Alternatively, as is known to those skilled in the art, the chiral auxiliary may be chemically removed to provide the pure enantiomer, after which the racemate may be covalently bonded to a chiral compound (auxiliary) to produce diastereomers which can be separated by chromatography or fractional crystallization.

In the present invention, the term “tautomer” is a type of structural isomer, which is a type of structural isomer of a compound that is easily converted into another compound by tautomerization in which rearrangement of hydrogen atoms occurs. A chemical equilibrium is achieved due to the rearrangement of hydrogen atoms. For example, there may be eno-keto, lactam-lactim, amide-eimidic acid, and amine-imine tautomers. In addition, in the case of heteroaromatic compounds such as triazole, tautomers may be formed due to the movement of hydrogen atoms.

In one embodiment of the present invention, the compound of Chemical Formula 1 provided by the present invention includes its salt form, and the salt may preferably be in the form of a pharmaceutically acceptable salt. In the present invention, the term “pharmaceutically acceptable” refers to properties that do not impair the biological activity and physical properties of the compound. In the present invention, the pharmaceutically acceptable salt refers to a compound group represented by Chemical Formula 1, which is pharmaceutically acceptable and has the desired pharmacological activity as defined above. Examples of such salts include, but are not limited to, acid addition salts formed with inorganic acids (e.g., hydrochloric acid, hydrobromic acid, sulfuric acid, phosphoric acid, nitric acid, etc.), and salts formed with organic acids such as acetic acid, oxalic acid, tartaric acid, succinic acid, malic acid, fumaric acid, maleic acid, ascorbic acid, benzoic acid, tannic acid, pamoic acid, alginic acid, polyglutamic acid, naphthalene sulfonic acid, naphthalene disulfonic acid, and poly-galacturonic acid. The compound may also be administered in the form of pharmaceutically acceptable quaternary salts known to those skilled in the art, particularly chloride, bromide, iodide, —O-alkyl, toluenesulfonate, methylsulfonate, sulfonate, phosphate, or carboxylate (e.g., benzoate, succinate, acetate, glycolate, maleate, malate, fumarate, citrate, tartrate, ascorbate, cinnamate, mandelate, and diphenylacetate).

The present invention also provides a pharmaceutical composition for preventing or treating cancer or degenerative neurological diseases, comprising the compound or a pharmaceutically acceptable salt thereof as an active ingredient.

Additionally, the present invention also provides a pharmaceutical composition for preventing or treating cancer or degenerative neurological diseases, consisting of the compound or a pharmaceutically acceptable salt thereof.

Furthermore, the present invention also provides a pharmaceutical composition for preventing or treating cancer or degenerative neurological diseases, consisting essentially of the compound or a pharmaceutically acceptable salt thereof.

In the present invention, the cancer is not particularly limited in type but may be selected from the group consisting of blood cancer, multiple myeloma, acute myeloid leukemia, malignant lymphoma, aplastic anemia, thymic cancer, ovarian cancer, cervical cancer, breast cancer, colorectal cancer, liver cancer, stomach cancer, pancreatic cancer, colon cancer, peritoneal metastatic cancer, skin cancer, bladder cancer, prostate cancer, thyroid cancer, lung cancer, osteosarcoma, fibrous tumor, and brain tumor.

In the present invention, the degenerative neurological disease is not particularly limited but may be selected from the group consisting of cognitive dysfunction, dementia, Alzheimer's disease, Parkinson's disease, Lou Gehrig's disease, Huntington's disease, amyotrophic lateral sclerosis, multiple sclerosis, immune system abnormality brain dysfunction, progressive neurodegenerative disease, metabolic encephalopathy, Niemann-Pick disease, Pick's disease, dementia due to cerebral ischemia, and dementia due to cerebral hemorrhage.

The pharmaceutical composition according to the invention may contain the compound of Chemical Formula 1 or a pharmaceutically acceptable salt thereof alone or may further contain one or more pharmaceutically acceptable carriers, excipients, or diluents.

Pharmaceutically acceptable carriers may include, for example, carriers for oral administration or carriers for parenteral administration. Carriers for oral administration may include lactose, starch, cellulose derivatives, magnesium stearate, stearic acid, and the like. Additionally, carriers for parenteral administration may include water, suitable oils, saline, aqueous glucose, and glycols, and may further include stabilizers and preservatives. Suitable stabilizers include antioxidants such as sodium bisulfite, sodium sulfite, or ascorbic acid. Suitable preservatives include benzalkonium chloride, methyl- or propyl-paraben, and chlorobutanol. Other pharmaceutically acceptable carriers are known in the art.

The pharmaceutical composition of the present invention can be administered to mammals, including humans, by any method. For example, it can be administered orally or parenterally. Parenteral administration methods include, but are not limited to, intravenous, intramuscular, intra-arterial, intramedullary, intradural, intracardiac, transdermal, subcutaneous, intraperitoneal, intranasal, enteral, topical, sublingual, or rectal administration. For example, the pharmaceutical composition of the present invention can be administered by preparing it in an injectable form and lightly pricking the skin with a fine needle of 30 gauge, or by directly applying it to the skin.

The pharmaceutical composition of the present invention can be formulated for oral or parenteral administration according to the administration route described above.

In the case of oral formulations, the composition of the present invention can be formulated into powders, granules, tablets, pills, sugar-coated tablets, capsules, liquids, gels, syrups, slurries, suspensions, etc., using methods known in the art. For example, oral formulations can be obtained by mixing the active ingredient with a solid excipient, grinding it, adding suitable auxiliaries, and processing it into a granulated mixture to obtain tablets or sugar-coated tablets. Suitable excipients include sugars such as lactose, dextrose, sucrose, sorbitol, mannitol, xylitol, erythritol, and maltitol, starches such as corn starch, wheat starch, rice starch, and potato starch, celluloses such as cellulose, methylcellulose, sodium carboxymethylcellulose, and hydroxypropylmethyl-cellulose, and fillers such as gelatin and polyvinylpyrrolidone. Additionally, cross-linked polyvinylpyrrolidone, agar, alginic acid, or sodium alginate can be added as disintegrants. Furthermore, the pharmaceutical composition of the present invention may further include anti-caking agents, lubricants, wetting agents, flavoring agents, emulsifiers, and preservatives.

In the case of parenteral formulations, they can be formulated into injections, creams, lotions, external ointments, oils, moisturizers, gels, aerosols, and nasal inhalants using methods known in the art. These formulations are generally known in all pharmaceutical chemistry.

The total effective amount of the pharmaceutical composition of the present invention can be administered to a patient as a single dose or as multiple doses over a long period in a fractionated treatment protocol. The pharmaceutical composition of the present invention may vary in the content of the active ingredient depending on the severity of the disease. Preferably, the total dose of the pharmaceutical composition of the present invention may be about 0.01 ug to 1,000 mg per kg of patient body weight per day, more preferably 0.1 ug to 100 mg. However, the dosage of the pharmaceutical composition of the present invention is determined by considering various factors such as the administration route, frequency of treatment, age, weight, health condition, gender, severity of the disease, diet, and excretion rate of the patient, so an effective dose for the patient is determined accordingly. Considering these factors, those skilled in the art can determine the appropriate effective dose of the pharmaceutical composition of the present invention for specific uses as a treatment for degenerative neurological diseases. The pharmaceutical composition according to the present invention is not particularly limited in its formulation, administration route, and administration method as long as it exhibits the effects of the present invention.

The present invention also provides the use of the compound or a pharmaceutically acceptable salt thereof for preparing a pharmaceutical composition for treating cancer or a degenerative neurological disease.

The present invention provides a method for treating cancer or a degenerative neurological disease comprising administering an effective amount of a pharmaceutical composition comprising the compound or a pharmaceutically acceptable salt thereof as an active ingredient to a subject in need thereof.

The ‘effective amount’ of the present invention refers to an amount that, when administered to a subject, exhibits an effect of improving, treating, detecting, diagnosing, or inhibiting or reducing cancer or the disease, and the ‘subject’ may be an animal, preferably a mammal, particularly an animal including a human, and may also be cells, tissues, organs, etc., derived from an animal. The subject may be a patient in need of the effect.

The ‘treatment’ of the present invention comprehensively refers to improving symptoms caused by cancer or the disease, which may include curing the disease, substantially preventing it, or improving the condition, and includes alleviating, curing, or preventing one or most of the symptoms derived from the disease, but is not limited thereto.

The term “comprising” as used herein is used in the same sense as “including” or “characterized by,” and in the composition or method according to the present invention, it does not exclude additional components or steps of the method that are not specifically mentioned. The term “consisting of” refers to excluding additional elements, steps, or components that are not separately described. The term “consisting essentially of” refers to that the scope of the composition or method may include substances or steps that do not substantially affect the basic characteristics of the described substance or step.

### Effect of the Invention

The compound according to the present invention has excellent activity in inhibiting PLD, making it highly useful for the development of treatments for cancer and/or degenerative neurological diseases.

### BRIEF DESCRIPTION OF THE DRAWINGS

FIG. 1a to FIG. 1b show the results of evaluating the PLD inhibitory activity of the compound according to the present invention in vitro.

FIG. 2 shows the results of evaluating the cytotoxicity of the compound according to the present invention on colon cancer cell lines.

FIG. 3 shows the results of evaluating the effect of the compound according to the present invention on the cell cycle of normal cells and cancer cells, respectively.

FIG. 4 shows the results of evaluating the effect of the compound according to the present invention on apoptosis of normal cells and cancer cells, respectively.

FIG. 5a to FIG. 5d show the results of evaluating the anticancer activity of the compound according to the present invention in an inflammatory colon cancer animal model (AOM/DSS model).

FIG. 6a to FIG. 6b show the results of evaluating the anticancer activity of the compound according to the present invention in a colon cancer cell line orthotopic animal model (orthotopic model).

### DETAILED DESCRIPTION OF THE INVENTION

Hereinafter, the present invention will be described in detail with reference to the following examples. However, the following examples are merely illustrative of the present invention, and the present invention is not limited thereto.

Hereinafter, the present invention will be described in detail.

However, the following examples are merely illustrative of the present invention, and the content of the present invention is not limited to the following examples.

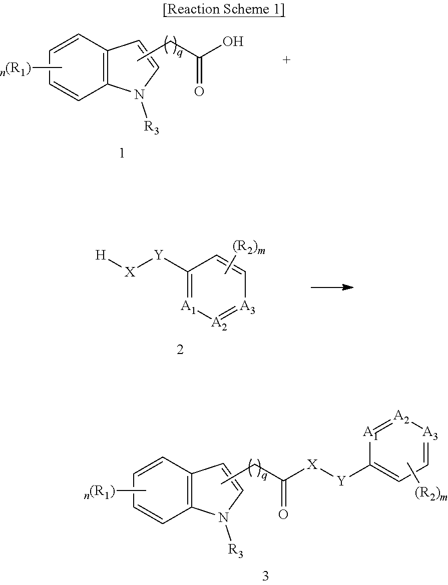

### Example 1. Preparation of Compounds

The following examples were prepared in the same manner as Examples 1-3 prepared according to the above Reaction Scheme 1.

### <Example 1-3> Preparation of N-(3,4-dimethoxyphenyl)-1H-indole-3-carboxamide

Indole-3-carboxylic acid (Compound 1, 1 eq) and 3,4-dimethoxyaniline (Compound 2, 1 eq) were added to anhydrous dichloromethane (DCM) with 1-ethyl-3-(3-dimethylaminopropyl)carbodiimide hydrochloride (EDC HCl, 2 eq), N,N-diisopropylethylamine (DIPEA, 3 eq), and 1-hydroxybenzotriazole hydrate (HOBt·H₂O, 2 eq), and stirred overnight at room temperature. After the reaction was completed, the reaction mixture was concentrated under reduced pressure, and the concentrate was purified by column chromatography (eluent: ethyl acetate/hexane 1:7 v/v mixture) to obtain the target compound of Example 1-3.

### <Example 1-1> Preparation of N-(4-chloro-3-nitrophenyl)-1H-indole-3-carboxamide

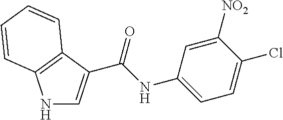 [SMILES: O=C(Nc1ccc(Cl)c([N+](=O)[O-])c1)c1c[nH]c2ccccc12]

The target compound was obtained by reacting in the same manner as in Example 1-3.

1H NMR (400 MHz, DMSO-d6) δ 11.83 (s, 1H), 10.20 (s, 1H), 8.58 (d, 1H, J=2.5 Hz), 8.31 (s, 1H), 8.16 (d, 1H, J=7.2 Hz), 8.01 (dd, 1H, J=8.9, 2.5 Hz), 7.70 (d, 1H, J=8.9 Hz), 7.46 (d, 1H, J=7.6 Hz), 7.21-7.11 (m, 2H). ESI (m/z) 314 (MH−).

### <Example 1-2> Preparation of N-(4-bromophenyl)-1H-indole-3-carboxamide

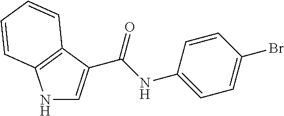 [SMILES: O=C(Nc1ccc(Br)cc1)c1c[nH]c2ccccc12]

The target compound was obtained by reacting in the same manner as in Example 1-3.

1H NMR (400 MHz, DMSO-d6) δ 11.74 (s, 1H), 9.81 (s, 1H), 8.27 (d, 1H, J=2.0 Hz), 8.17 (d, 1H, J=7.6 Hz), 7.75 (d, 2H, J=8.6 Hz), 7.57-7.38 (m, 3H), 7.22-7.03 (m, 2H). 13C NMR (400 MHz, DMSO-d6) δ 163.7, 139.6, 136.6, 131.7, 131.7, 129.3, 126.7, 122.6, 121.9, 121.9, 121.4, 121.2, 114.4, 112.4, 110.6. ESI (m/z) 313 (MH−), 315 (MH−).

### <Example 1-3> Preparation of N-(3,4-dimethoxyphenyl)-1H-indole-3-carboxamide

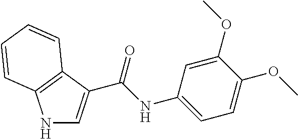 [SMILES: COc1ccc(NC(=O)c2c[nH]c3ccccc23)cc1OC]

The target compound was obtained by reacting in the same manner as in Example 1-3.

1H NMR (400 MHz, DMSO-d6) δ 11.67 (s, 1H), 9.56 (s, 1H), 8.24 (d, 1H, J=1.4 Hz), 8.18 (d, 1H, J=7.5 Hz), 7.46 (d, 1H, J=2.2 Hz), 7.44 (d, 1H, J=7.8 Hz), 7.28 (dd, 1H, J=8.7, 2.2 Hz), 7.17-7.08 (m, 2H), 6.88 (d, 1H, J=8.7 Hz), 3.74 (s, 3H), 3.70 (s, 3H). 13C NMR (400 MHz, DMSO-d6) δ 163.4, 148.9, 144.8, 136.6, 133.8, 128.7, 126.8, 122.5, 121.5, 121.0, 112.4, 112.3, 112.0, 111.0, 105.4, 56.1, 55.8. ESI (m/z) 297 (MH+), 319 (MNa+), 295 (MH−)

### <Example 1-4> Preparation of N-(3,4-dichlorophenyl)-1H-indole-3-carboxamide

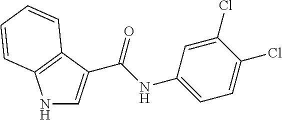 [SMILES: O=C(Nc1ccc(Cl)c(Cl)c1)c1c[nH]c2ccccc12]

The target compound was obtained by reacting in the same manner as in Example 1-3.

1H NMR (400 MHz, DMSO-d6) δ 11.78 (s, 1H), 9.94 (s, 1H), 8.27 (s, 1H), 8.17-8.11 (m, 2H), 7.70 (dd, 1H, J=8.8, 2.4 Hz), 7.55 (d, 1H, J=8.8 Hz), 7.45 (d, 1H, J=7.7 Hz), 7.20-7.09 (m, 2H). ESI (m/z) 303 (MH−).

### <Example 1-5> Preparation of N-(3,4-dichlorobenzyl)-1H-indole-3-carboxamide

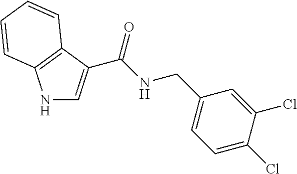 [SMILES: O=C(NCc1ccc(Cl)c(Cl)c1)c1c[nH]c2ccccc12]

The target compound was obtained by reacting in the same manner as in Example 1-3.

1H NMR (400 MHz, DMSO-d6) δ 11.58 (s, 1H), 8.51 (t, 1H, J=6.0 Hz), 8.12 (d, 1H, J=7.6 Hz), 8.05 (d, 1H, J=2.9 Hz), 7.58-7.52 (m, 1H), 7.41 (d, 1H, J=7.8 Hz), 7.31 (dd, 1H, J=8.4, 1.8 Hz), 7.15-7.10 (m, 1H), 7.10-7.05 (m, 1H), 4.44 (d, 2H, J=6.0 Hz). 13C NMR (400 MHz, DMSO-d6) δ 165.1, 142.2, 136.5, 131.2, 130.8, 129.5, 129.4, 128.4, 128.0, 126.5, 122.3, 121.3, 120.8, 112.2, 110.5, 41.3. ESI (m/z) 317 (MH−).

### <Example 1-6> Preparation of N′-(3,4-dichlorophenyl)-1H-indole-2-carbohydrazide

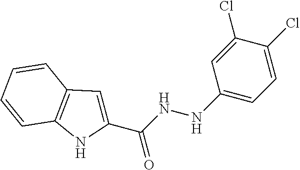 [SMILES: O=C(NNc1ccc(Cl)c(Cl)c1)c1cc2ccccc2[nH]1]

The target compound was obtained by reacting in the same manner as in Example 1-3.

1H NMR (400 MHz, DMSO-d6) δ 11.70 (s, 1H), 10.46 (s, 1H), 8.41 (s, 1H), 7.62 (d, 1H, J=8.0 Hz), 7.42 (d, 1H, J=8.2 Hz), 7.35 (d, 1H, J=8.8 Hz), 7.26 (s, 1H), 7.18 (t, 1H, J=7.5 Hz), 7.03 (t, 1H, J=7.4 Hz), 6.90 (s, 1H), 6.75 (d, 1H, J=8.8 Hz). 13C NMR (400 MHz, DMSO-d6) δ 161.7, 150.1, 137.1, 131.7, 131.1, 129.8, 127.4, 124.1, 122.1, 120.4, 119.8, 113.4, 112.9, 112.8, 103.7. ESI (m/z) 342 (MNa+), 318 (MH−).

### <Example 1-7> Preparation of N-(2-hydroxyphenyl)-2-(1H-indol-3-yl)acetamide

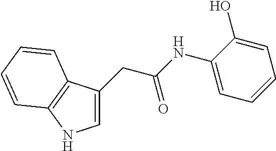 [SMILES: O=C(Cc1c[nH]c2ccccc12)Nc1ccccc1O]

The target compound was obtained by reacting in the same manner as in Example 1-3.

1H NMR (400 MHz, DMSO-d6) δ 10.94 (s, 1H), 9.69 (s, 1H), 9.05 (s, 1H), 7.81 (d, J=7.8 Hz, 1H), 7.56 (d, J=7.8 Hz, 1H), 7.33 (d, J=8.0 Hz, 1H), 7.29 (s, 1H), 7.05 (t, J=7.5 Hz, 1H), 6.95 (t, J=7.4 Hz, 1H), 6.85 (t, J=7.4 Hz, 1H), 6.75 (d, J=7.8 Hz, 1H), 6.70 (t, J=7.5 Hz, 1H), 3.79 (s, 2H). ESI (m/z) 267 (MH+), 289 (MNa+).

### <Example 1-8> Preparation of N-(3-hydroxyphenyl)-2-(1H-indol-3-yl)acetamide

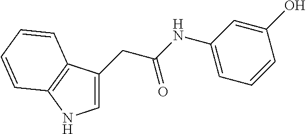 [SMILES: O=C(Cc1c[nH]c2ccccc12)Nc1cccc(O)c1]

The target compound was obtained by reacting in the same manner as in Example 1-3.

1H NMR (400 MHz, DMSO-d6) δ 10.99 (s, 1H), 7.55 (d, 1H, J=7.9 Hz), 7.12-7.05 (m, 1H), 7.02-6.93 (m, 2H), 6.38 (dd, 1H, J=8.0, 1.3 Hz), 6.20 (t, 1H, J=2.1 Hz), 6.15 (dd, 1H, J=7.9, 1.5 Hz), 5.24 (s, 2H), 3.93 (s, 2H). 13C NMR (400 MHz, DMSO-d6) δ 170.7, 151.9, 150.4, 136.5, 129.9, 127.4, 124.7, 121.5, 119.0, 118.8, 111.9, 111.6, 108.7, 107.1, 107.0, 31.3. ESI (m/z) 267 (MH+), 289 (MNa+).

### <Example 1-9> Preparation of 2-(1H-indol-3-yl)-N-(2-methoxyphenyl)acetamide

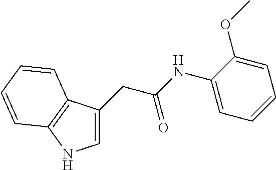 [SMILES: COc1ccccc1NC(=O)Cc1c[nH]c2ccccc12]

The target compound was obtained by reacting in the same manner as in Example 1-3.

1H NMR (400 MHz, DMSO-d6) δ 10.96 (s, 1H), 8.98 (s, 1H), 7.97 (d, 1H, J=7.3 Hz), 7.59 (d, 1H, J=7.9 Hz), 7.35 (d, 1H, J=8.1 Hz), 7.29 (s, 1H), 7.06 (t, 1H, J=7.2 Hz), 7.02-6.92 (m, 3H), 6.87-6.81 (m, 1H), 3.80 (s, 2H), 3.68 (s, 3H). 13C NMR (400 MHz, DMSO-d6) δ 170.1, 149.3, 136.6, 127.8, 127.5, 124.6, 124.4, 121.5, 121.2, 120.7, 119.1, 118.9, 111.8, 111.4, 108.8, 56.0, 34.0. ESI (m/z) 281 (MH+), 282 (MNa+)

### <Example 1-10> Preparation of 2-(1H-indol-3-yl)-N-(3-methoxyphenyl)acetamide

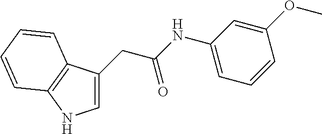 [SMILES: COc1cccc(NC(=O)Cc2c[nH]c3ccccc23)c1]

The target compound was obtained by reacting in the same manner as in Examples 1-3.

1H NMR (400 MHz, DMSO-d6) δ 10.90 (s, 1H), 10.08 (s, 1H), 7.57 (d, J=7.8 Hz, 1H), 7.35-7.29 (m, 2H), 7.23 (d, J=1.4 Hz, 1H), 7.18-7.10 (m, 2H), 7.04 (t, J=7.5 Hz, 1H), 6.95 (t, J=7.4 Hz, 1H), 6.57 (d, J=8.8 Hz, 1H), 3.69 (s, 2H), 3.67 (s, 3H). ESI (m/z) 281 (MH+), 282 (MNa+).

### <Example 1-11> Preparation of 2-(1H-indol-3-yl)-N-(4-methoxyphenyl)acetamide

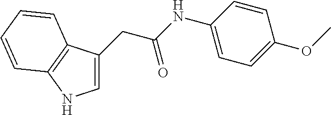 [SMILES: COc1ccc(NC(=O)Cc2c[nH]c3ccccc23)cc1]

The target compound was obtained by reacting in the same manner as in Examples 1-3.

1H NMR (400 MHz, DMSO-d6) δ 10.89 (s, 1H), 9.95 (s, 1H), 7.58 (d, 1H, J=7.8 Hz), 7.49 (d, 2H, J=8.8 Hz), 7.32 (d, 1H, J=8.0 Hz), 7.22 (s, 1H), 7.04 (t, 1H, J=7.3 Hz), 6.95 (t, 1H, J=7.4 Hz), 6.83 (d, 2H, J=8.8 Hz), 3.66 (s, 5H). 13C NMR (400 MHz, DMSO-d6) δ 169.6, 155.4, 136.5, 132.9, 127.6, 124.2, 121.4, 120.9, 120.9, 119.1, 118.7, 114.1, 114.1, 111.7, 109.1, 55.5, 34.1. ESI (m/z) 281 (MH+), 282 (MNa+).

### <Example 1-12> Preparation of N-(2-fluorophenyl)-2-(1H-indol-3-yl)acetamide

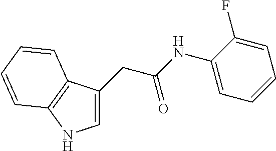 [SMILES: O=C(Cc1c[nH]c2ccccc12)Nc1ccccc1F]

The target compound was obtained by reacting in the same manner as in Examples 1-3.

1H NMR (400 MHz, DMSO-d6) δ 10.92 (s, 1H), 9.81 (s, 1H), 7.90-7.81 (m, 1H), 7.59 (d, 1H, J=7.8 Hz), 7.33 (d, 1H, J=8.1 Hz), 7.27-7.16 (m, 2H), 7.13-7.01 (m, 3H), 6.96 (t, 1H, J=7.4 Hz), 3.79 (s, 2H). ESI (m/z) 269 (MH+), 291 (MNa+).

### <Example 1-13> Preparation of N-(3-fluorophenyl)-2-(1H-indol-3-yl)acetamide

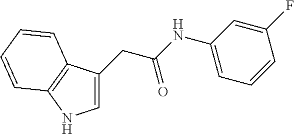 [SMILES: O=C(Cc1c[nH]c2ccccc12)Nc1cccc(F)c1]

The target compound was obtained by reacting in the same manner as in Examples 1-3.

1H NMR (400 MHz, DMSO-d6) δ 10.92 (s, 1H), 10.32 (s, 1H), 7.63-7.53 (m, 2H), 7.34-7.22 (m, 4H), 7.04 (d, 1H, J=7.6 Hz), 6.96 (t, 1H, J=7.4 Hz), 6.85-6.77 (m, 1H), 3.71 (s, 2H). ESI (m/z) 269 (MH+), 291 (MNa+).

### <Example 1-14> Preparation of N-(4-fluorophenyl)-2-(1H-indol-3-yl)acetamide

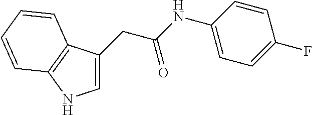 [SMILES: O=C(Cc1c[nH]c2ccccc12)Nc1ccc(F)cc1]

The target compound was obtained by reacting in the same manner as in Examples 1-3.

1H NMR (400 MHz, DMSO-d6) δ 10.90 (s, 1H), 10.15 (s, 1H), 7.62-7.56 (m, 3H), 7.32 (d, 1H, J=8.1 Hz), 7.23 (d, 1H, J=2.1 Hz), 7.13-7.01 (m, 3H), 6.98-6.92 (m, 1H), 3.69 (s, 2H). ESI (m/z) 269 (MH+), 291 (MNa+).

### <Example 1-15> Preparation of N-(2-bromophenyl)-2-(1H-indol-3-yl)acetamide

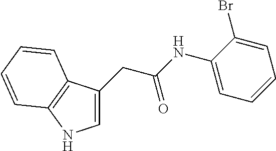 [SMILES: O=C(Cc1c[nH]c2ccccc12)Nc1ccccc1Br]

The target compound was obtained by reacting in the same manner as in Examples 1-3.

1H NMR (400 MHz, DMSO-d6) δ 10.96 (s, 1H), 9.29 (s, 1H), 7.71 (d, 1H, J=7.9 Hz), 7.58 (t, 2H, J=7.1 Hz), 7.36-7.27 (m, 3H), 7.09-7.01 (m, 2H), 6.96 (t, 1H, J=7.4 Hz), 3.79 (s, 2H). ESI (m/z) 329 (MH+), 331 (MH+), 351 (MNa−), 353 (MNa−)

### <Example 1-16> Preparation of N-(3-bromophenyl)-2-(1H-indol-3-yl)acetamide

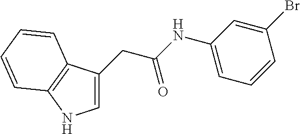 [SMILES: O=C(Cc1c[nH]c2ccccc12)Nc1cccc(Br)c1]

The target compound was obtained by reacting in the same manner as in Examples 1-3.

1H NMR (400 MHz, DMSO-d6) δ 10.92 (s, 1H), 10.27 (s, 1H), 7.95 (t, 1H, J=1.7 Hz), 7.56 (d, 1H, J=7.8 Hz), 7.50-7.46 (m, 1H), 7.32 (d, 1H, J=8.1 Hz), 7.25-7.16 (m, 3H), 7.07-7.01 (m, 1H), 6.98-6.92 (m, 1H), 3.71 (s, 2H). 13C NMR (400 MHz, DMSO-d6) δ 170.5, 141.3, 136.5, 131.1, 127.5, 126.0, 124.3, 121.9, 121.7, 121.4, 119.0, 118.8, 118.1, 111.8, 108.6, 34.2. ESI (m/z) 329 (MH+), 331 (MH+), 351 (MNa−), 353 (MNa−).

### <Example 1-17> Preparation of N-(4-bromophenyl)-2-(1H-indol-3-yl)acetamide

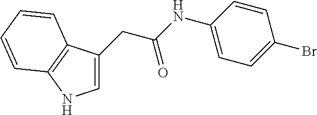 [SMILES: O=C(Cc1c[nH]c2ccccc12)Nc1ccc(Br)cc1]

The target compound was obtained by reacting in the same manner as in Examples 1-3.

1H NMR (400 MHz, DMSO-d6) δ 10.91 (s, 1H), 10.23 (s, 1H), 7.56 (d, 3H, J=8.7 Hz), 7.44 (d, 2H, J=8.8 Hz), 7.32 (d, 1H, J=8.1 Hz), 7.23 (d, 1H, J=1.7 Hz), 7.04 (t, 1H, J=7.3 Hz), 6.95 (t, 1H, J=7.4 Hz), 3.70 (s, 2H). 13C NMR (400 MHz, DMSO-d6) δ170.3, 139.1, 136.5, 131.9, 131.9, 127.5, 124.3, 121.4, 121.3, 121.3, 119.0, 118.8, 114.9, 111.8, 108.7, 34.2. ESI (m/z) 329 (MH+), 331 (MH+), 351 (MNa−), 353 (MNa−).

### <Example 1-18> Preparation of N-(3,5-difluorophenyl)-2-(1H-indol-3-yl)acetamide

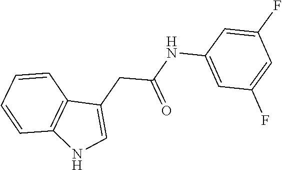 [SMILES: O=C(Cc1c[nH]c2ccccc12)Nc1cc(F)cc(F)c1]

The target compound was obtained by reacting in the same manner as in Examples 1-3.

1H NMR (400 MHz, DMSO-d6) δ 10.91 (s, 1H), 10.45 (s, 1H), 7.54 (d, 1H, J=7.9 Hz), 7.34-7.28 (m, 3H), 7.23 (d, 1H, J=1.8 Hz), 7.04 (t, 1H, J=7.5 Hz), 6.95 (t, 1H, J=7.4 Hz), 6.85 (t, 1H, J=9.4 Hz), 3.71 (s, 2H). ESI (m/z) 285 (MH−).

### <Example 1-19> Preparation of N-(4-chloro-3-nitrophenyl)-2-(1H-indol-3-yl)acetamide

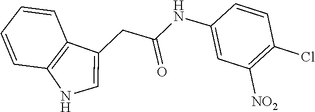 [SMILES: O=C(Cc1c[nH]c2ccccc12)Nc1ccc(Cl)c([N+](=O)[O-])c1]

The target compound was obtained by reacting in the same manner as in Examples 1-3.

1H NMR (400 MHz, DMSO-d6) δ 10.93 (s, 1H), 10.64 (s, 1H), 8.41 (d, 1H, J=2.2 Hz), 7.79 (dd, 1H, J=8.9, 2.3 Hz), 7.65 (d, 1H, J=8.8 Hz), 7.56 (d, 1H, J=7.9 Hz), 7.33 (d, 1H, J=8.1 Hz), 7.26 (s, 1H), 7.05 (t, 1H, J=7.5 Hz), 6.96 (t, 1H, J=7.4 Hz), 3.75 (s, 2H). 13C NMR (400 MHz, DMSO-d6) δ 171.0, 147.6, 139.6, 136.5, 132.4, 127.5, 124.5, 124.3, 121.4, 119.0, 118.9, 118.6, 115.7, 111.8, 108.2, 34.2. ESI (m/z) 328 (MH−).

### <Example 1-20> Preparation of N-(3,4-dichlorophenyl)-2-(1H-indol-3-yl)acetamide

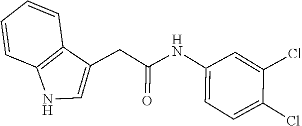 [SMILES: O=C(Cc1c[nH]c2ccccc12)Nc1ccc(Cl)c(Cl)c1]

The target compound was obtained by reacting in the same manner as in Examples 1-3.

1H NMR (400 MHz, DMSO-d6) δ 10.92 (s, 1H), 10.38 (s, 1H), 7.99 (d, 1H, J=1.4 Hz), 7.56 (d, 1H, J=7.8 Hz), 7.53-7.47 (m, 2H), 7.33 (d, 1H, J=8.1 Hz), 7.24 (d, 1H, J=1.5 Hz), 7.05 (t, 1H, J=7.5 Hz), 6.96 (t, 1H, J=7.4 Hz), 3.72 (s, 2H). 13C NMR (400 MHz, DMSO-d6) δ 170.6, 139.8, 136.5, 131.3, 131.0, 127.5, 124.8, 124.4, 121.4, 120.6, 119.4, 119.0, 118.9, 111.8, 108.4, 34.2. ESI (m/z) 317 (MH−), 318 (MH−).

### <Example 1-21> Preparation of N-(5-bromopyridin-2-yl)-2-(1H-indol-3-yl)acetamide

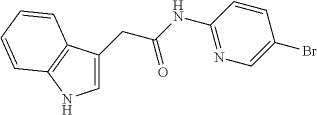 [SMILES: O=C(Cc1c[nH]c2ccccc12)Nc1ccc(Br)cn1]

The target compound was obtained by reacting in the same manner as in Examples 1-3.

1H NMR (400 MHz, DMSO-d6) δ 10.90 (s, 1H), 10.72 (s, 1H), 8.39 (d, 1H, J=2.5 Hz), 8.03 (d, 1H, J=8.9 Hz), 7.93 (dd, 1H, J=8.9, 2.5 Hz), 7.57 (d, 1H, J=7.9 Hz), 7.32 (d, 1H, J=8.1 Hz), 7.25 (d, 1H, J=1.7 Hz), 7.04 (t, 1H, J=7.5 Hz), 6.95 (t, 1H, J=7.4 Hz), 3.79 (s, 2H). 13C NMR (400 MHz, DMSO-d6) δ171.1, 151.5, 148.8, 140.9, 136.5, 127.5, 124.5, 121.4, 119.1, 118.8, 115.3, 113.6, 111.8, 108.4, 33.9. ESI (m/z) 330 (MH+), 332 (MH+), 328 (MH−), 330 (MH−).

### <Example 1-22> Preparation of N-(5-fluoropyridin-2-yl)-2-(1H-indol-3-yl)acetamide

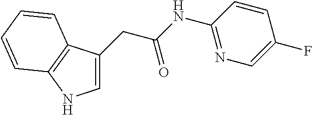 [SMILES: O=C(Cc1c[nH]c2ccccc12)Nc1ccc(F)cn1]

The target compound was obtained by reacting in the same manner as in Examples 1-3.

1H NMR (400 MHz, DMSO-d6) δ 10.89 (s, 1H), 10.63 (s, 1H), 8.27 (d, 1H, J=2.5 Hz), 8.07 (dd, 1H, J=9.1, 4.0 Hz), 7.67 (td, 1H, J=8.9, 2.7 Hz), 7.57 (d, 1H, J=7.9 Hz), 7.32 (d, 1H, J=8.1 Hz), 7.25 (s, 1H), 7.03 (t, 1H, J=7.5 Hz), 6.94 (t, 1H, J=7.4 Hz), 3.77 (s, 2H). ESI (m/z) 270 (MH+), 268 (MH−).

### <Example 1-23> Preparation of 2-(1H-indol-3-yl)-N-phenylacetamide

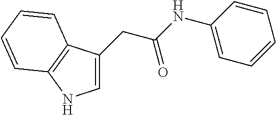 [SMILES: O=C(Cc1c[nH]c2ccccc12)Nc1ccccc1]

The target compound was obtained by reacting in the same manner as in Examples 1-3.

1H NMR (400 MHz, DMSO-d6) δ 10.91 (s, 1H), 10.09 (s, 1H), 7.61 (d, 3H, J=7.6 Hz), 7.35 (d, 1H, J=7.6 Hz), 7.27 (d, 2H, J=8.4 Hz), 7.26 (s, 1H), 7.06 (t, 1H, J=7.0 Hz), 7.00 (t, 1H, J=8.0 Hz), 6.98 (t, 1H, J=7.6 Hz), 3.73 (s, 2H). 13C NMR (400 MHz, DMSO-d6) δ 170.2, 139.8, 136.6, 129.1, 129.1, 127.7, 124.3, 123.5, 121.4, 119.5, 119.5, 119.1, 118.8, 111.8, 109.0, 34.3. ESI (m/z) 251 (MH+), 273 (MNa+).

### <Example 1-24> Preparation of N-(4-hydroxyphenyl)-2-(1H-indol-3-yl)acetamide

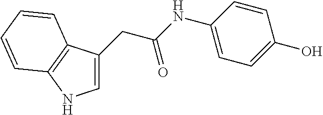 [SMILES: O=C(Cc1c[nH]c2ccccc12)Nc1ccc(O)cc1]

The target compound was obtained by reacting in the same manner as in Examples 1-3.

1H NMR (400 MHz, DMSO-d6) δ 10.86 (s, 1H), 9.80 (s, 1H), 9.13 (s, 1H), 7.60 (d, 1H, J=8.0 Hz), 7.36 (d, 2H, J=8.8 Hz), 7.33 (d, 1H, J=8.8 Hz), 7.22 (s, 1H), 7.05 (t, 1H, J=7.4 Hz), 6.96 (t, 1H, J=7.2 Hz), 6.67 (d, 2H, J=8.8 Hz), 3.66 (s, 2H). 13C NMR (400 MHz, DMSO-d6) δ 169.5, 153.6, 136.5, 131.5, 127.7, 124.2, 121.4, 121.3, 121.3, 119.2, 118.8, 115.4, 115.4, 111.8, 109.3, 34.1. ESI (m/z) 267 (MH+), 289 (MNa+).

### <Example 1-25> Preparation of methyl 4-(2-(1H-indol-3-yl)acetamido)benzoate

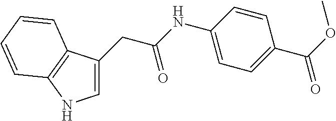 [SMILES: COC(=O)c1ccc(NC(=O)Cc2c[nH]c3ccccc23)cc1]

The target compound was obtained by reacting in the same manner as in Examples 1-3.

1H NMR (400 MHz, DMSO-d6) δ 10.91 (s, 1H), 10.42 (s, 1H), 7.88 (d, 2H, J=7.6 Hz), 7.74 (d, 2H, J=8.0 Hz), 7.58 (d, 1H, J=7.6 Hz), 7.34 (d, 1H, J=8.0 Hz), 7.25 (s, 1H), 7.05 (t, 1H, J=7.4 Hz), 6.96 (t, 1H, J=7.2 Hz), 3.78 (s, 3H), 3.76 (s, 2H). 13C NMR (400 MHz, DMSO-d6) δ 170.8, 166.2, 144.2, 136.5, 130.7, 130.7, 127.6, 124.4, 124.2, 121.5, 119.1, 118.9, 118.8, 118.8, 111.8, 108.6, 52.3, 34.4. ESI (m/z) 309 (MH+), 331 (MNa+).

### <Example 1-26> Preparation of 2-(1H-indol-3-yl)-N-(4-(trifluoromethyl)phenyl)acetamide

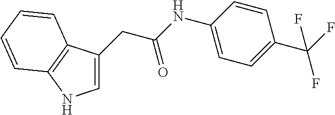 [SMILES: O=C(Cc1c[nH]c2ccccc12)Nc1ccc(C(F)(F)F)cc1]

The target compound was obtained by reacting in the same manner as in Examples 1-3.

1H NMR (400 MHz, DMSO-d6) δ 10.92 (s, 1H), 10.45 (s, 1H), 7.81 (d, 2H, J=8.4 Hz), 7.63 (d, 2H, J=8.4 Hz), 7.58 (d, 1H, J=8.0 Hz), 7.34 (d, 1H, J=8.0 Hz), 7.26 (s, 1H), 7.05 (t, 1H, J=7.4 Hz), 6.96 (t, 1H, J=7.4 Hz), 3.76 (s, 2H). ESI (m/z) 319 (MH+).

### <Example 1-27> Preparation of N-(4-tert-butylphenyl)-2-(1H-indol-3-yl)acetamide

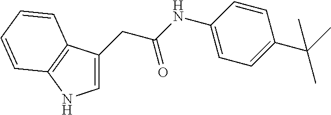 [SMILES: CC(C)(C)c1ccc(NC(=O)Cc2c[nH]c3ccccc23)cc1]

The target compound was obtained by reacting in the same manner as in Examples 1-3.

1H NMR (400 MHz, DMSO-d6) δ 10.88 (s, 1H), 10.00 (s, 1H), 7.59 (d, 1H, J=7.2 Hz), 7.51 (d, 2H, J=8.4 Hz), 7.34 (d, 1H, J=7.6 Hz), 7.27 (d, 2H, J=8.0 Hz), 7.23 (s, 1H), 7.05 (t, 1H, J=7.2 Hz), 6.96 (t, 1H, J=7.0 Hz), 3.70 (s, 2H), 1.22 (s, 9H). 13C NMR (400 MHz, DMSO-d6) δ 169.9, 145.7, 137.3, 136.6, 127.7, 125.7, 125.7, 124.3, 121.4, 119.3, 119.3, 119.1, 118.8, 111.8, 109.1, 34.4, 34.2, 31.6, 31.6, 31.6. ESI (m/z) 307 (MH+), 329 (MNa+).

### <Example 1-28> Preparation of N-(3,4-dimethoxyphenyl)-2-(1H-indol-3-yl)acetamide

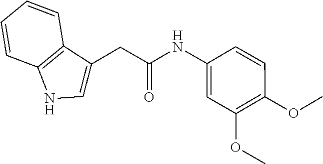 [SMILES: COc1ccc(NC(=O)Cc2c[nH]c3ccccc23)cc1OC]

The target compound was obtained by reacting in the same manner as in Examples 1-3.

1H NMR (400 MHz, DMSO-d6) δ 10.89 (s, 1H), 9.94 (s, 1H), 7.59 (d, 1H, J=7.8 Hz), 7.36-7.29 (m, 2H), 7.23 (s, 1H), 7.12-7.01 (m, 2H), 6.96 (t, 1H, J=7.4 Hz), 6.83 (d, 1H, J=8.7 Hz), 3.69-3.65 (m, 7H). 13C NMR (400 MHz, DMSO-d6) δ 169.7, 148.9, 145.0, 136.5, 133.5, 127.6, 124.2, 121.4, 119.1, 118.8, 112.4, 111.7, 111.2, 109.1, 104.6, 56.1, 55.7, 34.1. ESI (m/z) 311 (MH+), 333 (MNa+), 309 (MH−).

### <Example 1-29> Preparation of ethyl 4-(2-(1H-indol-3-yl)acetamido)benzoate

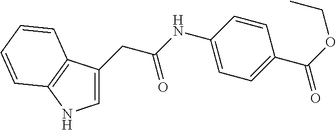 [SMILES: CCOC(=O)c1ccc(NC(=O)Cc2c[nH]c3ccccc23)cc1]

The target compound was obtained by reacting in the same manner as in Examples 1-3.

1H NMR (400 MHz, DMSO-d6) δ 10.91 (s, 1H), 10.42 (s, 1H), 7.88 (d, 2H, J=8.8 Hz), 7.74 (d, 2H, J=8.8 Hz), 7.58 (d, 1H, J=7.6 Hz), 7.34 (d, 1H, J=8.0 Hz), 7.25 (s, 1H), 7.05 (t, 1H, J=7.2 Hz), 6.96 (t, 1H, J=7.2 Hz), 4.24 (q, 2H, J=8.4 Hz), 3.76 (s, 2H), 1.26 (t, 3H, J=7.2 Hz). 13C NMR (400 MHz, DMSO-d6) δ 170.7, 165.8, 144.2, 136.5, 130.6, 130.6, 127.6, 124.5, 124.4, 121.5, 119.1, 118.9, 118.8, 118.8, 111.8, 108.6, 60.8, 34.3, 14.6. ESI (m/z) 323 (MH+), 345 (MNa+).

### <Example 1-30> Preparation of 2-(1H-indol-3-yl)-N-(3-nitrophenyl)acetamide

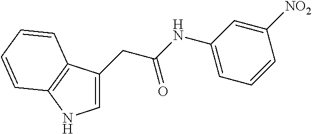 [SMILES: O=C(Cc1c[nH]c2ccccc12)Nc1cccc([N+](=O)[O-])c1]

The target compound was obtained by reacting in the same manner as in Examples 1-3.

1H NMR (400 MHz, DMSO-d6) δ 10.92 (s, 1H), 10.57 (s, 1H), 8.62 (s, 1H), 7.92 (d, 1H, J=8.0 Hz), 7.85 (d, 1H, J=8.0 Hz), 7.58 (d, 1H, J=8.8 Hz), 7.55 (t, 1H, J=8.4 Hz), 7.34 (d, 1H, J=8.0 Hz), 7.27 (s, 1H), 7.05 (t, 1H, J=7.2 Hz), 6.97 (t, 1H, J=7.2 Hz), 3.77 (s, 2H). 13C NMR (400 MHz, DMSO-d6) δ170.9, 148.4, 140.9, 136.6, 130.5, 127.6, 125.4, 124.5, 121.5, 119.0, 118.9, 118.0, 113.5, 111.8, 108.4, 34.3. ESI (m/z) 296 (MH+), 318 (MNa+).

### <Example 1-31> Preparation of N-(4-ethynylphenyl)-2-(1H-indol-3-yl)acetamide

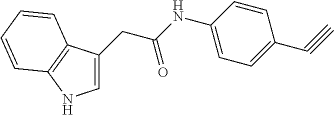 [SMILES: C#Cc1ccc(NC(=O)Cc2c[nH]c3ccccc23)cc1]

The target compound was obtained by reacting in the same manner as in Examples 1-3.

1H NMR (400 MHz, DMSO-d6) δ 10.90 (s, 1H), 10.25 (s, 1H), 7.55 (d, 1H, J=8.0 Hz), 7.50 (dd, 4H, J=8.2 Hz, 93.8 Hz), 7.34 (d, 1H, J=8.0 Hz), 7.24 (s, 1H), 7.05 (t, 1H, J=7.4 Hz), 6.96 (t, 1H, J=7.4 Hz), 4.03 (s, 1H), 3.73 (s, 2H). 13C NMR (400 MHz, DMSO-d6) δ 170.4, 140.4, 136.5, 132.8, 132.8, 127.6, 124.4, 121.5, 119.3, 119.3, 119.1, 118.9, 116.4, 111.8, 108.8, 84.0, 80.2, 34.3. ESI (m/z) 275 (MH+), 297 (MNa+).

### <Example 1-32> Preparation of N-(3,5-bis(trifluoromethyl)phenyl)-2-(1H-indol-3-yl)acetamide

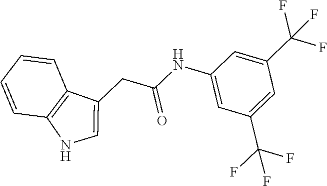 [SMILES: O=C(Cc1c[nH]c2ccccc12)Nc1cc(C(F)(F)F)cc(C(F)(F)F)c1]

The target compound was obtained by reacting in the same manner as in Examples 1-3.

1H NMR (400 MHz, DMSO-d6) δ 10.94 (s, 1H), 10.72 (s, 1H), 8.27 (s, 2H), 7.68 (s, 1H), 7.57 (d, 1H, J=7.6 Hz), 7.34 (d, 1H, J=8.0 Hz), 7.28 (s, 1H), 7.05 (t, 1H, J=7.6 Hz), 6.96 (t, 1H, J=7.4 Hz), 3.78 (s, 2H). ESI (m/z) 387 (MH+).

### <Example 1-33> Preparation of 2-(1H-indol-3-yl)-N-(4-isopropylphenyl)acetamide

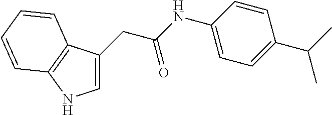 [SMILES: CC(C)c1ccc(NC(=O)Cc2c[nH]c3ccccc23)cc1]

The target compound was obtained by reacting in the same manner as in Examples 1-3.

1H NMR (400 MHz, DMSO-d6) δ 10.89 (s, 1H), 9.99 (s, 1H), 7.60 (d, 1H, J=7.6 Hz), 7.51 (d, 2H, J=8.0 Hz), 7.34 (d, 1H, J=8.0 Hz), 7.24 (s, 1H), 7.12 (d, 2H, J=8.0 Hz), 7.05 (t, 1H, J=7.4 Hz), 6.97 (t, 1H, J=7.2 Hz), 3.71 (s, 2H), 2.81-2.75 (m, 1H), 1.13 (d, 6H, J=7.2 Hz). 13C NMR (400 MHz, DMSO-d6) δ169.9, 143.5, 137.6, 136.6, 127.7, 126.8, 126.8, 124.3, 121.4, 119.6, 119.6, 119.1, 118.8, 111.8, 109.1, 34.2, 33.3, 24.4, 24.4. ESI (m/z) 293 (MH+), 315 (MNa+).

### <Example 1-34> Preparation of N-(4-sec-butylphenyl)-2-(1H-indol-3-yl)acetamide

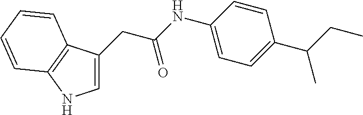 [SMILES: CCC(C)c1ccc(NC(=O)Cc2c[nH]c3ccccc23)cc1]

The target compound was obtained by reacting in the same manner as in Examples 1-3.

1H NMR (400 MHz, DMSO-d6) δ 10.88 (s, 1H), 9.99 (s, 1H), 7.60 (d, 1H, J=8.0 Hz), 7.51 (d, 2H, J=8.0 Hz), 7.34 (d, 1H, J=8.0 Hz), 7.24 (s, 1H), 7.07 (d, 2H, J=8.0 Hz), 7.05 (t, 1H, J=7.2 Hz), 6.97 (t, 1H, J=7.2 Hz), 3.70 (s, 2H), 2.53-2.42 (m, 1H), 1.52-1.43 (m, 2H), 1.22 (d, 3H, J=6.0 Hz), 0.71 (t, 3H, J=6.6 Hz). 13C NMR (400 MHz, DMSO-d6) δ 169.9, 142.2, 137.6, 136.6, 127.7, 127.4, 127.4, 124.3, 121.4, 119.6, 119.6, 119.1, 118.8, 111.8, 109.1, 40.8, 34.2, 31.0, 22.3, 12.5. ESI (m/z) 307 (MH+), 329 (MNa+).

### <Example 1-35> Preparation of N-(2-fluorobenzyl)-2-(1H-indol-3-yl)acetamide

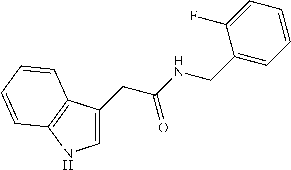 [SMILES: O=C(Cc1c[nH]c2ccccc12)NCc1ccccc1F]

The target compound was obtained by reacting in the same manner as in Examples 1-3.

1H NMR (400 MHz, DMSO-d6) δ 10.88 (s, 1H), 8.41 (t, 1H, J=5.6 Hz), 7.53 (d, 1H, J=8.0 Hz), 7.32 (d, 1H, J=5.2 Hz), 7.26 (d, 1H, J=5.2 Hz), 7.24 (t, 1H, J=6.2 Hz), 7.18 (d, 1H, J=2.0 Hz), 7.14 (d, 1H, J=10.0 Hz), 7.09 (t, 1H, J=6.0 Hz), 7.05 (t, 1H, J=8.8 Hz), 6.94 (t, 1H, J=7.4 Hz), 4.29 (d, 2H, J=5.6 Hz), 3.57 (s, 2H). ESI (m/z) 283 (MH+), 305 (MNa+).

### <Example 1-36> Preparation of N-(3-fluorobenzyl)-2-(1H-indol-3-yl)acetamide

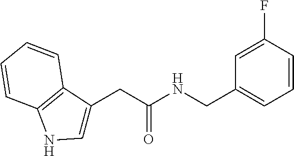 [SMILES: O=C(Cc1c[nH]c2ccccc12)NCc1cccc(F)c1]

The target compound was obtained by reacting in the same manner as in Examples 1-3.

1H NMR (400 MHz, DMSO-d6) δ 10.89 (s, 1H), 8.44 (t, 1H, J=5.6 Hz), 7.53 (d, 1H, J=8.0 Hz), 7.33 (d, 1H, J=8.0 Hz), 7.27 (t, 1H, J=7.4 Hz), 7.19 (d, 1H, J=1.6 Hz), 7.06 (d, 1H, J=7.6 Hz), 7.05 (s, 1H), 7.02 (d, 1H, J=8.4 Hz), 7.01 (t, 1H, J=20.8 Hz), 6.94 (t, 1H, J=7.4 Hz), 4.26 (d, 2H, J=6.0 Hz), 3.57 (s, 2H). ESI (m/z) 283 (MH+), 305 (MNa+).

### <Example 1-37> Preparation of N-(4-fluorobenzyl)-2-(1H-indol-3-yl)acetamide

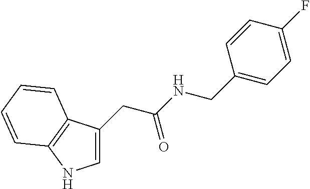 [SMILES: O=C(Cc1c[nH]c2ccccc12)NCc1ccc(F)cc1]

The target compound was obtained by reacting in the same manner as in Examples 1-3.

1H NMR (400 MHz, DMSO-d6) δ 10.87 (s, 1H), 8.41 (t, 1H, J=5.6 Hz), 7.51 (d, 1H, J=8.0 Hz), 7.32 (d, 1H, J=8.0 Hz), 7.24 (d, 1H, J=6.0 Hz), 7.22 (d, 1H, J=6.0 Hz), 7.17 (d, 2H, J=1.6 Hz), 7.08 (d, 1H, J=7.6 Hz), 7.05 (t, 1H, J=3.4 Hz), 7.03 (d, 1H, J=7.6 Hz), 6.94 (t, 1H, J=7.2 Hz), 4.22 (d, 2H, J=6.0 Hz), 3.55 (s, 2H). 13C NMR (400 MHz, DMSO-d6) δ 171.2, 162.7, 160.3, 136.5, 136.3, 136.3, 129.6, 129.6, 127.6, 124.3, 121.4, 119.1, 118.7, 115.4, 115.2, 111.7, 109.2, 41.9, 33.1. ESI (m/z) 283 (MH+), 305 (MNa+).

### <Example 1-38> Preparation of methyl 4-((2-(1H-indol-3-yl)acetamido)methyl)benzoate

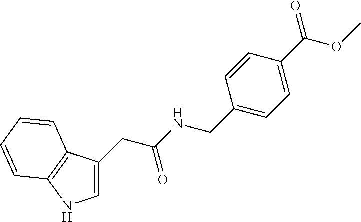 [SMILES: COC(=O)c1ccc(CNC(=O)Cc2c[nH]c3ccccc23)cc1]

The target compound was obtained by reacting in the same manner as in Examples 1-3.

1H NMR (400 MHz, DMSO-d6) δ 10.89 (s, 1H), 8.48 (t, 1H, J=5.8 Hz), 7.84 (d, 2H, J=8.4 Hz), 7.53 (d, 1H, J=7.6 Hz), 7.33 (d, 1H, J=8.8 Hz), 7.32 (d, 2H, J=8.0 Hz), 7.19 (d, 1H, J=1.6 Hz), 7.05 (t, 1H, J=7.6 Hz), 6.95 (t, 1H, J=7.4 Hz), 4.31 (d, 2H, J=6.0 Hz), 3.80 (s, 3H), 3.58 (s, 2H). 13C NMR (400 MHz, DMSO-d6) δ 171.4, 166.5, 145.8, 136.5, 129.5, 129.5, 128.4, 127.7, 127.7, 127.6, 124.3, 121.4, 119.0, 118.7, 111.7, 109.1, 52.4, 42.3, 33.1. ESI (m/z) 323 (MH+), 345 (MNa+).

### <Example 1-39> Preparation of 2-(1H-indol-3-yl)-N-(4-methoxybenzyl)acetamide

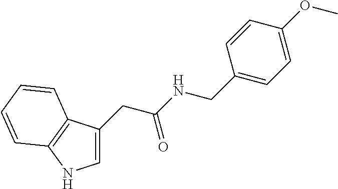 [SMILES: COc1ccc(CNC(=O)Cc2c[nH]c3ccccc23)cc1]

The target compound was obtained by reacting in the same manner as in Examples 1-3.

1H NMR (400 MHz, DMSO-d6) δ 10.87 (s, 1H), 8.33 (t, 1H, J=5.7 Hz), 7.52 (d, 1H, J=7.8 Hz), 7.32 (d, 1H, J=8.1 Hz), 7.18-7.10 (m, 3H), 7.04 (t, 1H, J=7.4 Hz), 6.94 (t, 1H, J=7.4 Hz), 6.81 (d, 2H, J=8.5 Hz), 4.17 (d, 2H, J=5.8 Hz), 3.68 (s, 3H), 3.53 (s, 2H). 13C NMR (400 MHz, DMSO-d6) δ 171.0, 158.5, 136.5, 131.9, 129.0, 129.0, 127.6, 124.2, 121.3, 119.1, 118.6, 114.0, 114.0, 111.7, 109.3, 55.4, 42.1, 33.1. ESI (m/z) 295 (MH+), 317 (MNa+).

### <Example 1-40> Preparation of 2-(1H-indol-3-yl)-N-(3-methylbenzyl)acetamide

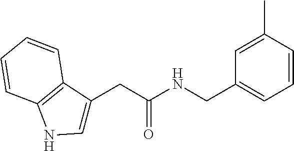 [SMILES: Cc1cccc(CNC(=O)Cc2c[nH]c3ccccc23)c1]

The target compound was obtained by reacting in the same manner as in Examples 1-3.

1H NMR (400 MHz, DMSO-d6) δ 10.88 (s, 1H), 8.36 (t, 1H, J=5.7 Hz), 7.56 (d, 1H, J=7.9 Hz), 7.33 (d, 1H, J=8.1 Hz), 7.19 (s, 1H), 7.13 (t, 1H, J=7.5 Hz), 7.05 (t, 1H, J=7.4 Hz), 7.02-6.92 (m, 4H), 4.21 (d, 2H, J=5.9 Hz), 3.56 (s, 2H), 2.18 (s, 3H). 13C NMR (400 MHz, DMSO-d6) δ 171.2, 139.9, 137.7, 136.6, 128.5, 128.1, 127.7, 127.6, 124.7, 124.3, 121.4, 119.2, 118.7, 111.7, 109.3, 42.5, 33.2, 21.4. ESI (m/z) 279 (MH+), 301 (MNa+).

### <Example 1-41> Preparation of N-(4-tert-butylbenzyl)-2-(1H-indol-3-yl)acetamide

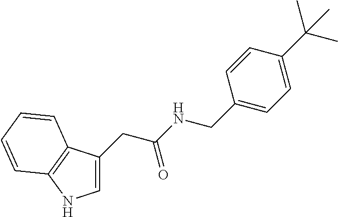 [SMILES: CC(C)(C)c1ccc(CNC(=O)Cc2c[nH]c3ccccc23)cc1]

The target compound was obtained by reacting in the same manner as in Examples 1-3.

1H NMR (400 MHz, DMSO-d6) δ 10.86 (s, 1H), 8.33 (t, 1H, J=5.7 Hz), 7.54 (d, 1H, J=7.9 Hz), 7.33 (d, 1H, J=8.1 Hz), 7.28 (d, 2H, J=8.3 Hz), 7.19 (d, 1H, J=2.2 Hz), 7.14 (d, 2H, J=8.3 Hz), 7.06 (t, 1H, J=8.0 Hz), 6.96 (t, 1H, J=8.0 Hz), 4.21 (d, 2H, J=5.8 Hz), 3.55 (s, 2H), 1.24 (s, 9H). 13C NMR (400 MHz, DMSO-d6) δ 171.0, 160.0, 158.1, 136.5, 129.2, 127.6, 124.2, 121.3, 119.4, 119.1, 118.6, 111.7, 109.4, 104.5, 98.5, 55.7, 55.6, 37.6, 33.0, 33.0, 33.0. ESI (m/z) 321 (MH+).

### <Example 1-42> Preparation of 2-(1H-indol-3-yl)-N-(3-(trifluoromethyl)benzyl)acetamide

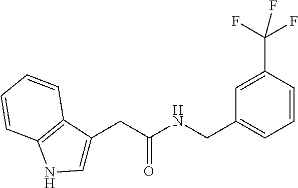 [SMILES: O=C(Cc1c[nH]c2ccccc12)NCc1cccc(C(F)(F)F)c1]

The target compound was obtained by reacting in the same manner as in Examples 1-3.

1H NMR (400 MHz, DMSO-d6) δ 10.86 (s, 1H), 8.46 (t, 1H, J=5.2 Hz), 7.55-7.47 (m, 5H), 7.31 (d, 1H, J=8.4 Hz), 7.17 (s, 1H), 7.03 (t, 1H, J=7.4 Hz), 6.92 (t, 1H, J=7.4 Hz), 4.32 (d, 2H, J=6.0 Hz), 3.56 (s, 2H). ESI (m/z) 333 (MH+), 355 (MNa+).

### <Example 1-43> Preparation of 2-(1H-indol-3-yl)-N-(4-(trifluoromethyl)benzyl)acetamide

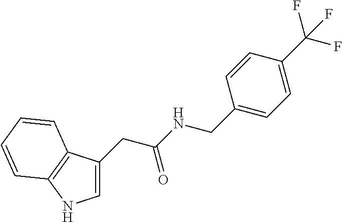 [SMILES: O=C(Cc1c[nH]c2ccccc12)NCc1ccc(C(F)(F)F)cc1]

The target compound was obtained by reacting in the same manner as in Examples 1-3.

1H NMR (400 MHz, DMSO-d6) δ 10.86 (s, 1H), 8.46 (t, 1H, J=5.4 Hz), 7.60 (d, 2H, J=8.0 Hz), 7.51 (d, 1H, J=7.6 Hz), 7.40 (d, 2H, J=8.0 Hz), 7.32 (d, 1H, J=8.0 Hz), 7.18 (d, 1H, J=2.0 Hz), 7.04 (t, 1H, J=7.2 Hz), 6.94 (t, 1H, J=7.0 Hz), 4.32 (d, 2H, J=6.0 Hz), 3.57 (s, 2H). ESI (m/z) 333 (MH+).

### <Example 1-44> Preparation of N-(2,4-dimethoxybenzyl)-2-(1H-indol-3-yl)acetamide

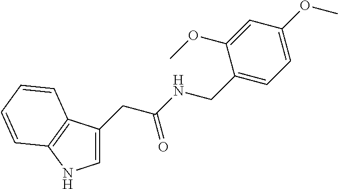 [SMILES: COc1ccc(CNC(=O)Cc2c[nH]c3ccccc23)c(OC)c1]

The target compound was obtained by reacting in the same manner as in Examples 1-3.

1H NMR (400 MHz, DMSO-d6) δ 10.84 (s, 1H), 8.01 (t, 1H, J=5.6 Hz), 7.52 (d, 1H, J=7.6 Hz), 7.32 (d, 1H, J=8.0 Hz), 7.16 (s, 1H), 7.04 (t, 1H, J=7.6 Hz), 7.00 (d, 1H, J=8.8 Hz), 6.94 (t, 1H, J=7.4 Hz), 6.49 (s, 1H), 6.38 (d, 1H, J=6.8 Hz), 4.12 (d, 2H, J=5.6 Hz), 3.69 (s, 6H), 3.53 (s, 2H). 13C NMR (400 MHz, DMSO-d6) δ 171.0, 149.0, 148.0, 136.5, 132.5, 127.6, 124.2, 121.3, 119.6, 119.1, 118.7, 112.0, 111.7, 111.3, 109.3, 55.9, 55.5, 42.3, 33.2. ESI (m/z) 325 (MH+), 347 (MNa+).

### <Example 1-45> Preparation of N-(3,4-dimethoxybenzyl)-2-(1H-indol-3-yl)acetamide

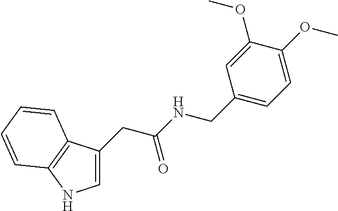 [SMILES: COc1ccc(CNC(=O)Cc2c[nH]c3ccccc23)cc1OC]

The target compound was obtained by reacting in the same manner as in Examples 1-3.

1H NMR (400 MHz, DMSO-d6) δ 10.87 (s, 1H), 8.31 (t, 1H, J=5.8 Hz), 7.57 (d, 1H, J=7.9 Hz), 7.33 (d, 1H, J=8.1 Hz), 7.19 (d, 1H, J=2.2 Hz), 7.05 (t, 1H, J=7.2 Hz), 6.95 (t, 1H, J=7.4 Hz), 6.82 (d, 1H, J=8.7 Hz), 6.75-6.69 (m, 2H), 4.19 (d, J=5.9 Hz, 2H), 3.68 (s, 3H), 3.55 (s, 2H), 3.54 (s, 3H). 13C NMR (400 MHz, DMSO-d6) δ 171.0, 149.0, 148.0, 136.5, 132.5, 127.6, 124.2, 121.3, 119.6, 119.1, 118.7, 112.0, 111.7, 111.3, 109.3, 55.9, 55.5, 42.3, 33.2. ESI (m/z) 325 (MH+), 347 (MNa+).

### <Example 1-46> Preparation of 2-(1H-indol-3-yl)-N-(4-nitrobenzyl)acetamide

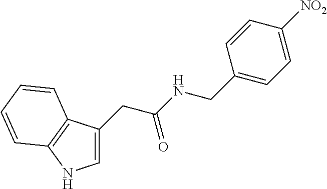 [SMILES: O=C(Cc1c[nH]c2ccccc12)NCc1ccc([N+](=O)[O-])cc1]

The target compound was obtained by reacting in the same manner as in Examples 1-3.

1H NMR (400 MHz, DMSO-d6) δ 10.86 (s, 1H), 8.49 (s, 1H), 8.10 (d, 2H, J=8.8 Hz), 7.52 (d, 1H, J=7.6 Hz), 7.44 (d, 2H, J=8.4 Hz), 7.33 (d, 1H, J=8.0 Hz), 7.19 (s, 1H), 7.05 (t, 1H, J=7.2 Hz), 6.95 (t, 1H, J=7.4 Hz), 4.36 (d, 2H, J=6.0 Hz), 3.57 (s, 2H). 13C NMR (400 MHz, DMSO-d6) δ 171.6, 148.3, 146.8, 136.6, 128.6, 128.6, 127.6, 124.4, 123.8, 123.8, 121.4, 119.0, 118.7, 111.8, 109.0, 42.3, 33.1. ESI (m/z) 308 (MH−).

### <Example 1-47> Preparation of N-(2,3-dichlorobenzyl)-2-(1H-indol-3-yl)acetamide

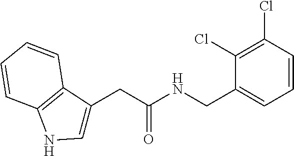 [SMILES: O=C(Cc1c[nH]c2ccccc12)NCc1cccc(Cl)c1Cl]

The target compound was obtained by reacting in the same manner as in Examples 1-3.

1H NMR (400 MHz, DMSO-d6) δ 10.87 (s, 1H), 8.43 (t, 1H, J=5.7 Hz), 7.54 (d, 1H, J=7.9 Hz), 7.48 (dd, 1H, J=6.9, 2.5 Hz), 7.33 (d, 1H, J=8.0 Hz), 7.25-7.18 (m, 3H), 7.05 (t, 1H, J=7.5 Hz), 6.95 (t, 1H, J=7.4 Hz), 4.33 (d, 2H, J=5.8 Hz), 3.60 (s, 2H). 13C NMR (400 MHz, DMSO-d6) δ 171.5, 139.6, 136.5, 132.0, 130.3, 129.3, 128.2, 127.6, 127.6, 124.3, 121.4, 119.0, 118.7, 111.7, 109.0, 41.3, 33.0. ESI (m/z) 355 (MNa+), 331 (MH−).

### <Example 1-48> Preparation of N-(2-chlorobenzyl)-2-(1H-indol-3-yl)acetamide

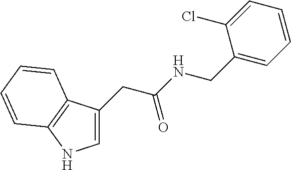 [SMILES: O=C(Cc1c[nH]c2ccccc12)NCc1ccccc1Cl]

The target compound was obtained by reacting in the same manner as in Examples 1-3.

1H NMR (400 MHz, DMSO-d6) δ 10.89 (s, 1H), 8.40 (t, 1H, J=5.6 Hz), 7.55 (d, 1H, J=8.0 Hz), 7.38 (d, 1H, J=5.2 Hz), 7.33 (d, 1H, J=8.0 Hz), 7.25-7.18 (m, 4H), 7.05 (t, 1H, J=7.4 Hz), 6.96 (t, 1H, J=7.2 Hz), 4.31 (d, 2H, J=5.6 Hz), 3.66 (s, 2H). 13C NMR (400 MHz, DMSO-d6) δ 171.4, 136.8, 136.5, 132.4, 129.4, 129.1, 128.9, 127.6, 127.4, 124.3, 121.4, 119.1, 118.7, 111.7, 109.1, 40.5, 33.0. ESI (m/z) 299 (MH+), 321 (MNa+).

### <Example 1-49> Preparation of N-(4-chlorobenzyl)-2-(1H-indol-3-yl)acetamide

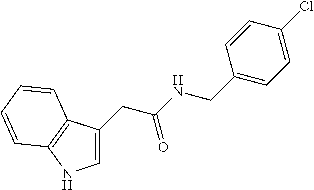 [SMILES: O=C(Cc1c[nH]c2ccccc12)NCc1ccc(Cl)cc1]

The target compound was obtained by reacting in the same manner as in Examples 1-3.

1H NMR (400 MHz, DMSO-d6) δ 10.88 (s, 1H), 8.42 (t, 1H, J=5.8 Hz), 7.51 (d, 1H, J=7.6 Hz), 7.32 (d, 2H, J=7.6 Hz), 7.19 (s, 1H), 7.21 (d, 2H, J=8.4 Hz), 7.18 (d, 1H, J=1.6 Hz), 7.05 (t, 1H, J=7.4 Hz), 6.94 (t, 1H, J=7.4 Hz), 4.22 (d, 2H, J=6.0 Hz), 3.55 (s, 2H). 13C NMR (400 MHz, DMSO-d6) δ 171.3, 139.1, 136.5, 131.6, 129.5, 129.5, 128.5, 128.5, 127.6, 124.2, 121.3, 119.1, 118.7, 111.7, 109.1, 41.9, 33.1. ESI (m/z) 299 (MH+), 321 (MNa+).

### <Example 1-50> Preparation of 2-(1H-indol-3-yl)-N-(2-methoxybenzyl)acetamide

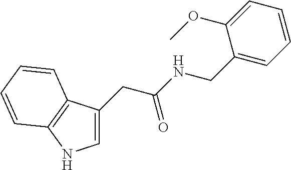 [SMILES: COc1ccccc1CNC(=O)Cc1c[nH]c2ccccc12]

The target compound was obtained by reacting in the same manner as in Examples 1-3.

1H NMR (400 MHz, DMSO-d6) δ 10.88 (s, 1H), 8.16 (t, 1H, J=5.8 Hz), 7.54 (d, 1H, J=8.0 Hz), 7.32 (d, 1H, J=8.0 Hz), 7.19 (d, 1H, J=5.2 Hz), 7.17 (t, 1H, J=7.4 Hz), 7.08 (d, 1H, J=7.2 Hz), 7.04 (t, 1H, J=7.6 Hz), 6.94 (t, 1H, J=7.4 Hz), 6.91 (d, 1H, J=8.0 Hz), 6.80 (t, 1H, J=7.4 Hz), 4.20 (d, 2H, J=5.6 Hz), 3.71 (s, 3H), 3.56 (s, 2H). 13C NMR (400 MHz, DMSO-d6) δ 171.2, 157.0, 136.5, 128.4, 128.1, 127.6, 127.2, 124.2, 121.3, 120.4, 119.1, 118.6, 111.7, 110.7, 109.3, 55.6, 37.8, 33.0. ESI (m/z) 295 (MH+), 317 (MNa+).

### <Example 1-51> Preparation of N′-(2-chlorophenyl)-2-(1H-indol-3-yl)acetohydrazide

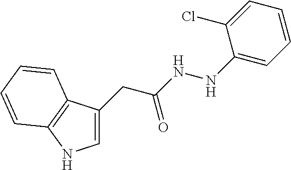 [SMILES: O=C(Cc1c[nH]c2ccccc12)NNc1ccccc1Cl]

The target compound was obtained by reacting in the same manner as in Examples 1-3.

1H NMR (400 MHz, DMSO-d6) δ 10.90 (s, 1H), 9.97 (d, 1H, J=1.4 Hz), 7.61 (d, 1H, J=7.8 Hz), 7.42 (d, 1H, J=9.3 Hz), 7.33 (d, 1H, J=8.1 Hz), 7.25-7.20 (m, 2H), 7.06 (t, 1H, J=7.5 Hz), 7.03-6.95 (m, 2H), 6.70-6.64 (m, 2H), 3.62 (s, 2H). 13C NMR (400 MHz, DMSO-d6) δ 170.9, 145.1, 136.4, 129.5, 128.0, 127.5, 124.3, 121.4, 119.7, 119.1, 118.7, 117.4, 113.2, 111.7, 108.5, 31.0. ESI (m/z) 300 (MH+), 322 (MNa+).

### <Example 1-52> Preparation of N′-(3-chlorophenyl)-2-(1H-indol-3-yl)acetohydrazide

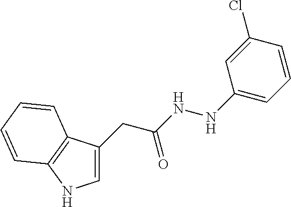 [SMILES: O=C(Cc1c[nH]c2ccccc12)NNc1cccc(Cl)c1]

The target compound was obtained by reacting in the same manner as in Examples 1-3.

1H NMR (400 MHz, DMSO-d6) δ 10.91 (s, 1H), 9.87 (d, 1H, J=2.0 Hz), 8.04 (d, 1H, J=2.0 Hz), 7.61 (d, 1H, J=7.8 Hz), 7.33 (d, 1H, J=8.1 Hz), 7.21 (d, 1H, J=1.8 Hz), 7.09-7.02 (m, 2H), 6.98 (t, 1H, J=7.3 Hz), 6.66-6.56 (m, 3H), 3.59 (s, 2H). 13C NMR (400 MHz, DMSO-d6) δ 171.0, 151.4, 136.5, 133.9, 130.6, 127.5, 124.3, 121.4, 119.0, 118.8, 118.1, 111.8, 111.6, 111.1, 108.6, 31.1. ESI (m/z) 300 (MH+), 322 (MNa+).

### <Example 1-53> Preparation of N′-(4-chlorophenyl)-2-(1H-indol-3-yl)acetohydrazide

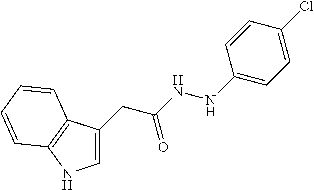 [SMILES: O=C(Cc1c[nH]c2ccccc12)NNc1ccc(Cl)cc1]

The target compound was obtained by reacting in the same manner as in Examples 1-3.

1H NMR (400 MHz, DMSO-d6) δ 10.88 (s, 1H), 9.83 (s, 1H), 7.90 (s, 1H), 7.60 (d, 1H, J=8.0 Hz), 7.34 (d, 1H, J=8.0 Hz), 7.21 (s, 1H), 7.08 (d, 2H, J=8.4 Hz), 7.07 (t, 1H, J=8.8 Hz), 6.98 (t, 1H, J=7.4 Hz), 6.65 (d, 2H, J=8.8 Hz), 3.58 (s, 2H). 13C NMR (400 MHz, DMSO-d6) δ 171.0, 148.8, 136.5, 128.8, 128.8, 127.6, 124.3, 122.0, 121.4, 119.1, 118.7, 114.0, 114.0, 111.8, 108.7, 31.1. ESI (m/z) 300 (MH+), 322 (MNa+).

### <Example 1-54> Preparation of N′-(3,4-dichlorophenyl)-2-(1H-indol-3-yl)acetohydrazide

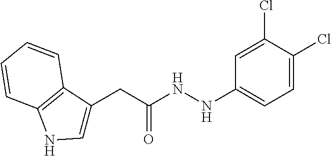 [SMILES: O=C(Cc1c[nH]c2ccccc12)NNc1ccc(Cl)c(Cl)c1]

The target compound was obtained by reacting in the same manner as in Examples 1-3.

1H NMR (400 MHz, DMSO-d6) δ 10.90 (s, 1H), 9.89 (s, 1H), 8.15 (s, 1H), 7.60 (d, 1H, J=7.8 Hz), 7.34 (d, 1H, J=8.1 Hz), 7.26 (d, 1H, J=8.8 Hz), 7.21 (s, 1H), 7.06 (t, 1H, J=7.4 Hz), 6.98 (t, 1H, J=7.4 Hz), 6.74 (s, 1H), 6.63 (d, 1H, J=8.7 Hz), 3.59 (s, 2H). 13C NMR (400 MHz, DMSO-d6) δ 171.1, 150.0, 136.5, 131.6, 130.8, 127.5, 124.3, 121.4, 119.5, 119.0, 118.8, 113.3, 112.9, 111.8, 108.5, 31.1. ESI (m/z) 332 (MH−).

### <Example 1-55> Preparation of N′-(4-bromophenyl)-2-(1H-indol-3-yl)acetohydrazide

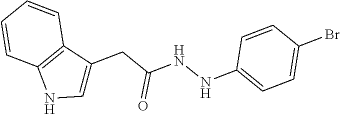 [SMILES: O=C(Cc1c[nH]c2ccccc12)NNc1ccc(Br)cc1]

The target compound was obtained by reacting in the same manner as in Examples 1-3.

1H NMR (400 MHz, DMSO-d6) δ 10.88 (s, 1H), 9.83 (s, 1H), 7.91 (s, 1H), 7.59 (d, 1H, J=7.8 Hz), 7.33 (d, 1H, J=8.0 Hz), 7.19 (d, 3H, J=8.6 Hz), 7.06 (t, 1H, J=7.4 Hz), 6.98 (t, 1H, J=7.4 Hz), 6.60 (d, 2H, J=8.3 Hz), 3.58 (s, 2H). ESI (m/z) 342 (MH−).

### <Example 1-56> Preparation of 2-(1H-indol-3-yl)-N-(pyridin-2-ylmethyl)acetamide

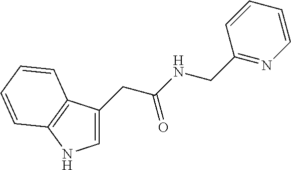 [SMILES: O=C(Cc1c[nH]c2ccccc12)NCc1ccccn1]

The target compound was obtained by reacting in the same manner as in Examples 1-3.

1H NMR (400 MHz, DMSO-d6) δ 10.88 (s, 1H), 8.59-8.37 (m, 2H), 7.65 (td, 1H, J=7.7, 1.5 Hz), 7.56 (d, 1H, J=7.8 Hz), 7.33 (d, 1H, J=8.0 Hz), 7.23-7.15 (m, 3H), 7.05 (t, 1H, J=7.5 Hz), 6.95 (t, 1H, J=7.4 Hz), 4.35 (d, 2H, J=5.9 Hz), 3.61 (s, 2H). 13C NMR (400 MHz, DMSO-d6) δ 171.4, 159.1, 149.1, 137.0, 136.5, 127.6, 124.3, 122.4, 121.3, 121.3, 119.1, 118.7, 111.7, 109.1, 44.7, 33.1. ESI (m/z) 266 (MH+), 288 (MNa+).

### <Example 1-57> Preparation of 2-(1H-indol-3-yl)-N-(pyridin-3-ylmethyl)acetamide

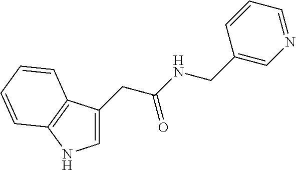 [SMILES: O=C(Cc1c[nH]c2ccccc12)NCc1cccnc1]

The target compound was obtained by reacting in the same manner as in Examples 1-3.

1H NMR (400 MHz, DMSO-d6) δ 10.88 (s, 1H), 8.50-8.43 (m, 2H), 8.40 (d, 1H, J=4.6 Hz), 7.58 (d, 1H, J=7.8 Hz), 7.51 (d, 1H, J=7.8 Hz), 7.33 (d, 1H, J=8.1 Hz), 7.27 (dd, 1H, J=7.8, 4.8 Hz), 7.18 (d, 1H, J=1.7 Hz), 7.05 (t, 1H, J=7.5 Hz), 6.94 (t, 1H, J=7.4 Hz), 4.27 (d, 2H, J=5.9 Hz), 3.57 (s, 2H). 13C NMR (400 MHz, DMSO-d6) δ 171.4, 149.1, 148.4, 136.5, 135.5, 135.4, 127.6, 124.3, 123.7, 121.4, 119.0, 118.7, 111.7, 109.1, 40.3, 33.1. ESI (m/z) 266 (MH+), 288 (MNa+), 264 (MH−).

### <Example 1-58> Preparation of 2-(1H-indol-3-yl)-N-(pyridin-4-ylmethyl)acetamide

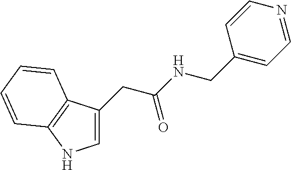 [SMILES: O=C(Cc1c[nH]c2ccccc12)NCc1ccncc1]

The target compound was obtained by reacting in the same manner as in Examples 1-3.

1H NMR (400 MHz, DMSO-d6) δ 10.89 (s, 1H), 8.45 (t, 1H, J=5.9 Hz), 8.42 (d, 2H, J=5.8 Hz), 7.54 (d, 1H, J=7.8 Hz), 7.33 (d, 1H, J=8.1 Hz), 7.20 (d, 1H, J=1.5 Hz), 7.17 (d, 2H, J=5.3 Hz), 7.05 (t, 1H, J=7.5 Hz), 6.96 (t, 1H, J=7.4 Hz), 4.26 (d, 2H, J=6.0 Hz), 3.59 (s, 2H). 13C NMR (400 MHz, DMSO-d6) δ 171.6, 149.8, 149.8, 149.1, 136.5, 127.6, 124.3, 122.5, 122.5, 121.4, 119.0, 118.7, 111.7, 109.0, 41.6, 33.0. ESI (m/z) 266 (MH+), 288 (MNa+), 264 (MH−).

### <Example 1-59> Preparation of 2-(1H-indol-3-yl)-N-phenethylacetamide

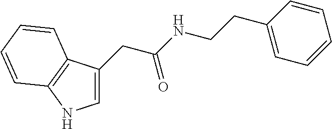 [SMILES: O=C(Cc1c[nH]c2ccccc12)NCCc1ccccc1]

The target compound was obtained by reacting in the same manner as in Examples 1-3.

1H NMR (400 MHz, DMSO-d6) δ 10.86 (s, 1H), 7.91 (t, 1H, J=5.0 Hz), 7.48 (d, 1H, J=8.0 Hz), 7.31 (d, 1H, J=5.0 Hz), 7.22 (t, 2H, J=6.8 Hz), 7.15 (t, 1H, J=6.8 Hz), 7.11 (d, 3H, J=8.0 Hz), 7.04 (t, 1H, J=7.4 Hz), 6.94 (t, 1H, J=7.4 Hz), 3.46 (s, 2H), 3.27-3.22 (m, 2H), 2.66 (t, 2H, J=7.2 Hz). 13C NMR (400 MHz, DMSO-d6) δ171.0, 139.9, 136.5, 129.1, 128.7, 127.6, 126.4, 124.2, 121.3, 119.1, 118.7, 111.7, 109.3, 40.8, 35.6, 33.2. ESI (m/z) 279 (MH+), 301 (MNa+).

### <Example 1-60> Preparation of N-(4-bromophenethyl)-2-(1H-indol-3-yl)acetamide

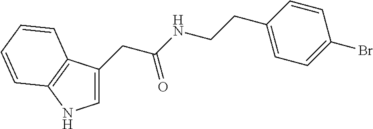 [SMILES: O=C(Cc1c[nH]c2ccccc12)NCCc1ccc(Br)cc1]

The target compound was obtained by reacting in the same manner as in Examples 1-3.

1H NMR (400 MHz, DMSO-d6) δ 10.84 (s, 1H), 7.84 (s, 1H), 7.46 (d, 1H, J=7.6 Hz), 7.36 (d, 2H, J=7.6 Hz), 7.33 (t, 1H, J=7.2 Hz), 7.14 (s, 1H), 7.04 (d, 3H, J=6.8 Hz), 6.94 (t, 1H, J=6.8 Hz), 3.45 (s, 2H), 3.24 (q, 2H, J=7.2 Hz), 2.63 (t, 2H, J=6.4 Hz). 13C NMR (400 MHz, DMSO-d6) δ 171.1, 139.4, 136.5, 131.5, 131.5, 131.4, 131.4, 127.6, 124.3, 121.3, 119.6, 119.1, 118.7, 111.7, 109.2, 40.4, 34.8, 33.2. ESI (m/z) 357 (MH+), 379 (MNa+).

### <Example 1-61> Preparation of N-(2-fluorophenyl)-3-(1H-indol-3-yl)propanamide

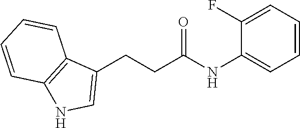 [SMILES: O=C(CCc1c[nH]c2ccccc12)Nc1ccccc1F]

The target compound was obtained by reacting in the same manner as in Examples 1-3.

1H NMR (400 MHz, DMSO-d6) δ 10.76 (s, 1H), 9.70 (s, 1H), 7.87 (d, 1H, J=6.8 Hz), 7.56 (d, 1H, J=7.6 Hz), 7.32 (d, 1H, J=8.0 Hz), 7.24-7.16 (m, 1H), 7.14-7.08 (m, 3H), 7.05 (t, 1H, J=7.6 Hz), 6.96 (t, 1H, J=7.4 Hz), 3.01 (t, 2H, J=7.4 Hz), 2.76 (t, 2H, J=7.2 Hz). 13C NMR (400 MHz, DMSO-d6) δ 171.8, 154.1, 136.7, 127.5, 126.7, 125.4, 124.7, 124.6, 122.6, 121.4, 118.8, 118.6, 115.8, 114.1, 111.8, 37.1, 21.3. ESI (m/z) 283 (MH+), 305 (MNa+).

### <Example 1-62> Preparation of N-(3-fluorophenyl)-3-(1H-indol-3-yl)propanamide

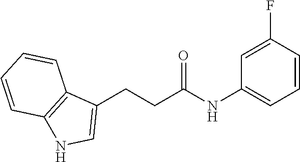 [SMILES: O=C(CCc1c[nH]c2ccccc12)Nc1cccc(F)c1]

The target compound was obtained by reacting in the same manner as in Examples 1-3.

1H NMR (400 MHz, DMSO-d6) δ 10.76 (s, 1H), 10.12 (s, 1H), 7.63 (d, 1H, J=12.0 Hz), 7.55 (d, 1H, J=7.6 Hz), 7.32 (d, 1H, J=8.0 Hz), 7.29 (t, 1H, J=5.2 Hz), 7.29 (s, 1H), 7.11 (s, 1H), 7.05 (t, 1H, J=7.4 Hz), 6.96 (t, 1H, J=7.4 Hz), 6.84-6.81 (m, 1H), 3.02 (t, 2H, J=7.6 Hz), 2.69 (t, 2H, J=7.4 Hz). 13C NMR (400 MHz, DMSO-d6) δ 171.8, 162.6, 141.5, 136.7, 130.7, 127.4, 122.6, 121.4, 118.8, 118.6, 115.2, 114.0, 111.8, 109.8, 106.2, 37.8, 21.1. ESI (m/z) 283 (MH+), 305 (MNa+).

### <Example 1-63> Preparation of N-(4-fluorophenyl)-3-(1H-indol-3-yl)propanamide

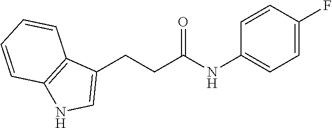 [SMILES: O=C(CCc1c[nH]c2ccccc12)Nc1ccc(F)cc1]

The target compound was obtained by reacting in the same manner as in Examples 1-3.

1H NMR (400 MHz, DMSO-d6) δ 10.75 (s, 1H), 9.95 (s, 1H), 7.60 (d, 1H, J=8.4 Hz), 7.59 (d, 1H, J=8.8 Hz), 7.55 (d, 1H, J=8.0 Hz), 7.31 (d, 1H, J=8.0 Hz), 7.11 (d, 2H, J=6.4 Hz), 7.10 (s, 1H), 7.04 (t, 1H, J=7.6 Hz), 6.95 (t, 1H, J=7.4 Hz), 3.01 (t, 2H, J=7.4 Hz), 2.66 (t, 2H, J=7.6 Hz). 13C NMR (400 MHz, DMSO-d6) δ171.3, 158.2, 136.7, 136.2, 127.4, 122.6, 121.4, 121.2, 121.1, 118.8, 118.6, 115.7, 115.5, 114.1, 111.8, 37.6, 21.2. ESI (m/z) 283 (MH+), 305 (MNa+).

### <Example 1-64> Preparation of N-(3-bromophenyl)-3-(1H-indol-3-yl)propanamide

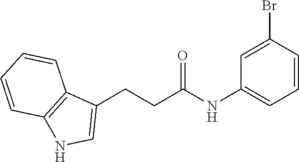 [SMILES: O=C(CCc1c[nH]c2ccccc12)Nc1cccc(Br)c1]

The target compound was obtained by reacting in the same manner as in Examples 1-3.

1H NMR (400 MHz, DMSO-d6) δ 10.75 (s, 1H), 10.07 (s, 1H), 7.97 (s, 1H), 7.54 (d, 1H, J=7.6 Hz), 7.47 (d, 1H, J=7.6 Hz), 7.32 (d, 1H, J=8.0 Hz), 7.22 (t, 1H, J=7.8 Hz), 7.20 (d, 1H, J=5.6 Hz), 7.11 (s, 1H), 7.04 (t, 1H, J=7.4 Hz), 6.96 (t, 1H, J=7.2 Hz), 3.01 (t, 2H, J=7.2 Hz), 2.68 (t, 2H, J=7.6 Hz). 13C NMR (400 MHz, DMSO-d6) δ 171.8, 141.3, 136.7, 131.1, 127.4, 126.0, 122.6, 122.0, 121.8, 121.4, 118.8, 118.6, 118.2, 114.0, 117.8, 37.8, 21.1. ESI (m/z) 343 (MH+), 345 (MH+), 365 (MNa−), 367 (MNa−).

### <Example 1-65> Preparation of N-(4-bromophenyl)-3-(1H-indol-3-yl)propanamide

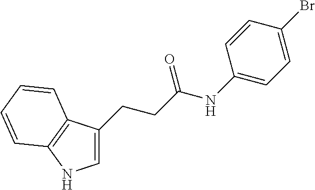 [SMILES: O=C(CCc1c[nH]c2ccccc12)Nc1ccc(Br)cc1]

The target compound was obtained by reacting in the same manner as in Examples 1-3.

1H NMR (400 MHz, DMSO-d6) δ 10.74 (s, 1H), 10.02 (s, 1H), 7.56 (d, 2H, J=8.8 Hz), 7.54 (d, 1H, J=8.4 Hz), 7.44 (d, 2H, J=8.8 Hz), 7.31 (d, 1H, J=8.0 Hz), 7.10 (s, 1H), 7.04 (t, 1H, J=7.2 Hz), 6.95 (t, 1H, J=7.2 Hz), 3.00 (t, 2H, J=7.6 Hz), 2.66 (t, 2H, J=7.6 Hz). 13C NMR (400 MHz, DMSO-d6) δ 171.6, 139.1, 136.7, 131.9, 131.9, 127.4, 122.6, 121.4, 121.4, 121.4, 118.8, 118.6, 114.9, 114.0, 111.8, 37.8, 21.2. ESI (m/z) 343 (MH+), 345 (MH+), 365 (MNa−), 367 (MNa−).

### <Example 1-66> Preparation of N-(3,5-difluorophenyl)-3-(1H-indol-3-yl)propanamide

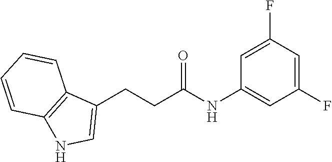 [SMILES: O=C(CCc1c[nH]c2ccccc12)Nc1cc(F)cc(F)c1]

The target compound was obtained by reacting in the same manner as in Examples 1-3.

1H NMR (400 MHz, DMSO-d6) δ 10.75 (s, 1H), 10.27 (s, 1H), 7.52 (d, 1H, J=7.8 Hz), 7.32-7.25 (m, 3H), 7.09 (d, 1H, J=1.8 Hz), 7.03 (t, 1H, J=7.4 Hz), 6.94 (t, 1H, J=7.4 Hz), 6.84 (tt, 1H, J=9.3, 2.1 Hz), 2.98 (t, 2H, J=7.5 Hz), 2.67 (t, 2H, J=7.6 Hz). ESI (m/z) 301 (MH+), 299 (MH−).

### <Example 1-67> Preparation of N-(3,5-bis(trifluoromethyl)phenyl)-3-(1H-indol-3-yl)propanamide

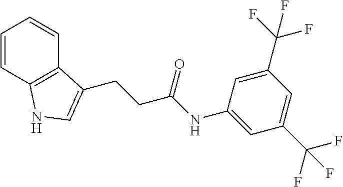 [SMILES: O=C(CCc1c[nH]c2ccccc12)Nc1cc(C(F)(F)F)cc(C(F)(F)F)c1]

The target compound was obtained by reacting in the same manner as in Examples 1-3.

1H NMR (400 MHz, DMSO-d6) δ 10.77 (s, 1H), 10.57 (s, 1H), 8.25 (s, 1H), 7.70 (s, 2H), 7.55 (d, 1H, J=7.8 Hz), 7.32 (d, 1H, J=8.1 Hz), 7.13 (d, 1H, J=1.7 Hz), 7.05 (t, 1H, J=7.5 Hz), 6.96 (t, 1H, J=7.4 Hz), 3.04 (t, 2H, J=7.5 Hz), 2.73 (t, 2H, J=7.5 Hz). ESI (m/z) 401 (MH+).

### <Example 1-68> Preparation of N-(4-chloro-3-nitrophenyl)-3-(1H-indol-3-yl)propanamide

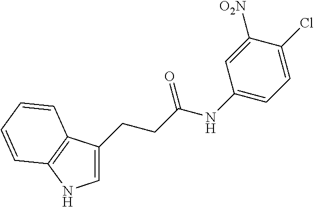 [SMILES: O=C(CCc1c[nH]c2ccccc12)Nc1ccc(Cl)c([N+](=O)[O-])c1]

The target compound was obtained by reacting in the same manner as in Examples 1-3.

1H NMR (400 MHz, DMSO-d6) δ 10.75 (s, 1H), 10.45 (s, 1H), 8.40 (d, 1H, J=2.4 Hz), 7.74 (dd, 1H, J=8.9, 2.4 Hz), 7.65 (d, 1H, J=8.8 Hz), 7.53 (d, 1H, J=7.8 Hz), 7.30 (d, 1H, J=8.1 Hz), 7.10 (d, 1H, J=1.8 Hz), 7.03 (t, 1H, J=7.5 Hz), 6.94 (t, 1H, J=7.4 Hz), 3.00 (t, 2H, J=7.5 Hz), 2.70 (t, 2H, J=7.6 Hz). 13C NMR (400 MHz, DMSO-d6) δ 172.2, 147.6, 139.5, 136.6, 132.3, 127.3, 124.2, 122.6, 121.3, 118.7, 118.6, 118.5, 115.6, 113.7, 111.8, 37.7, 20.9. ESI (m/z) 366 (MNa+), 342 (MH−).

### <Example 1-69> Preparation of N-(3,4-dichlorophenyl)-3-(1H-indol-3-yl)propanamide

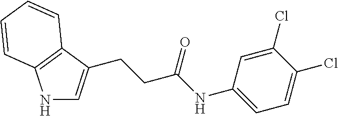 [SMILES: O=C(CCc1c[nH]c2ccccc12)Nc1ccc(Cl)c(Cl)c1]

The target compound was obtained by reacting in the same manner as in Examples 1-3.

1H NMR (400 MHz, DMSO-d6) δ 10.75 (s, 1H), 10.19 (s, 1H), 7.98 (d, 1H, J=2.2 Hz), 7.55-7.48 (m, 2H), 7.45 (dd, 1H, J=8.8, 2.3 Hz), 7.30 (d, 1H, J=8.1 Hz), 7.10 (d, 1H, J=1.7 Hz), 7.03 (t, 1H, J=7.5 Hz), 6.94 (t, 1H, J=7.4 Hz), 2.99 (t, 2H, J=7.5 Hz), 2.67 (t, 2H, J=7.6 Hz). 13C NMR (400 MHz, DMSO-d6) δ 171.9, 139.7, 136.6, 131.3, 131.0, 127.3, 124.7, 122.6, 121.3, 120.6, 119.4, 118.7, 118.6, 113.8, 111.7, 37.7, 21.0. ESI (m/z) 331 (MH−), 332 (MH−).

### <Example 1-70> Preparation of N-(3,4-dimethoxyphenyl)-3-(1H-indol-3-yl)propanamide

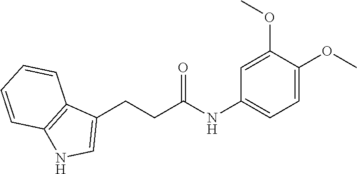 [SMILES: COc1ccc(NC(=O)CCc2c[nH]c3ccccc23)cc1OC]

The target compound was obtained by reacting in the same manner as in Examples 1-3.

1H NMR (400 MHz, DMSO-d6) δ 10.74 (s, 1H), 9.75 (s, 1H), 7.54 (d, 1H, J=7.8 Hz), 7.33-7.26 (m, 2H, J=9.8 Hz), 7.12-7.00 (m, 3H), 6.95 (t, 1H, J=7.4 Hz), 6.83 (d, 1H, J=8.7 Hz), 3.69 (s, 3H), 3.67 (s, 3H), 2.98 (t, 2H, J=7.5 Hz), 2.62 (t, 2H, J=7.6 Hz). 13C NMR (400 MHz, DMSO-d6) δ 170.9, 148.9, 145.0, 136.6, 133.4, 127.4, 122.5, 121.3, 118.7, 118.5, 114.1, 112.4, 111.7, 111.3, 104.7, 56.1, 55.7, 37.6, 21.3. ESI (m/z) 347 (MNa+), 323 (MH−).

### <Example 1-71> Preparation of 3-(1H-indol-3-yl)-N-(4-methoxybenzyl)propanamide

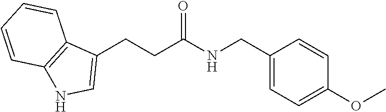 [SMILES: COc1ccc(CNC(=O)CCc2c[nH]c3ccccc23)cc1]

The target compound was obtained by reacting in the same manner as in Examples 1-3.

1H NMR (400 MHz, DMSO-d6) δ 10.78 (s, 1H), 8.27 (t, 1H, J=5.8 Hz), 7.54 (d, 1H, J=7.8 Hz), 7.34 (d, 1H, J=8.1 Hz), 7.10-7.03 (m, 4H), 6.99-6.93 (m, 1H), 6.85-6.80 (m, 2H), 4.19 (d, 2H, J=5.8 Hz), 3.70 (s, 3H), 2.96 (t, 2H, J=7.6 Hz), 2.50 (t, 2H, J=7.8 Hz). 13C NMR (400 MHz, DMSO-d6) δ 172.2, 158.5, 136.7, 131.9, 128.8, 128.8, 127.5, 122.6, 121.3, 118.8, 118.5, 114.2, 114.0, 114.0, 111.7, 55.4, 41.9, 36.7, 21.5. ESI (m/z) 309 (MH+), 331 (MNa+), 307 (MH−).

### <Example 1-72> Preparation of N-(3,4-dichlorobenzyl)-3-(1H-indol-3-yl)propanamide

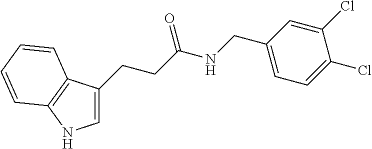 [SMILES: O=C(CCc1c[nH]c2ccccc12)NCc1ccc(Cl)c(Cl)c1]

The target compound was obtained by reacting in the same manner as in Examples 1-3.

1H NMR (400 MHz, DMSO-d6) δ 10.75 (s, 1H), 8.39 (t, 1H, J=5.7 Hz), 7.50 (d, 1H, J=7.9 Hz), 7.47 (d, 1H, J=8.3 Hz), 7.40 (s, 1H), 7.31 (d, 1H, J=8.0 Hz), 7.09-7.00 (m, 3H), 6.94 (t, 1H, J=7.4 Hz), 4.22 (d, 2H, J=5.9 Hz), 2.94 (t, 2H, J=7.5 Hz), 2.50 (t, 2H, J=7.5 Hz). 13C NMR (400 MHz, DMSO-d6) δ 172.5, 141.3, 136.6, 131.2, 130.7, 129.5, 129.4, 127.8, 127.4, 122.6, 121.3, 118.7, 118.5, 114.0, 111.7, 41.3, 36.6, 21.4. ESI (m/z) 345 (MH−), 346 (MH−).

### <Example 1-73> Preparation of N-(3,4-dimethoxybenzyl)-3-(1H-indol-3-yl)propanamide

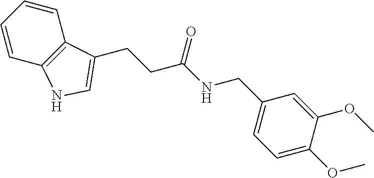 [SMILES: COc1ccc(CNC(=O)CCc2c[nH]c3ccccc23)cc1OC]

The target compound was obtained by reacting in the same manner as in Examples 1-3.

1H NMR (400 MHz, DMSO-d6) δ 10.75 (s, 1H), 8.26 (t, 1H, J=5.4 Hz), 7.51 (d, 1H, J=7.8 Hz), 7.31 (d, 1H, J=8.0 Hz), 7.08 (s, 1H), 7.04 (t, 1H, J=7.5 Hz), 6.94 (t, 1H, J=7.4 Hz), 6.84-6.78 (m, 2H), 6.67 (d, 1H, J=8.1 Hz), 4.16 (t, 2H, J=17.0 Hz), 3.69 (s, 3H), 3.65 (s, 3H), 2.94 (t, 2H, J=7.6 Hz), 2.50 (t, 2H, J=7.7 Hz). 13C NMR (400 MHz, DMSO-d6) δ 172.2, 149.0, 148.0, 136.6, 132.4, 127.4, 122.5, 121.3, 119.6, 118.8, 118.5, 114.2, 112.0, 111.7, 111.6, 55.9, 55.7, 42.2, 36.7, 21.5. ESI (m/z) 361 (MNa+), 337 (MH−).

### <Example 1-74> Preparation of N-(3-fluorophenyl)-4-(1H-indol-3-yl)butanamide

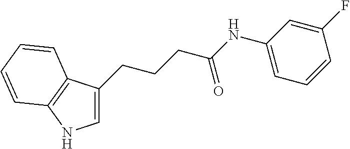 [SMILES: O=C(CCCc1c[nH]c2ccccc12)Nc1cccc(F)c1]

The target compound was obtained by reacting in the same manner as in Examples 1-3.

1H NMR (400 MHz, DMSO-d6) δ 10.75 (s, 1H), 10.06 (s, 1H), 7.61 (d, 1H, J=12.0 Hz), 7.50 (d, 1H, J=7.6 Hz), 7.32 (d, 1H, J=8.0 Hz), 7.28 (s, 1H), 7.28 (d, 1H, J=10.0 Hz), 7.10 (s, 1H), 7.04 (t, 1H, J=7.4 Hz), 6.94 (t, 1H, J=7.4 Hz), 6.83-6.80 (m, 1H), 2.72 (t, 2H, J=7.4 Hz), 2.36 (t, 2H, J=7.4 Hz), 1.99-1.92 (m, 2H). 13C NMR (400 MHz, DMSO-d6) δ 172.1, 162.6, 141.5, 136.8, 130.6, 127.6, 122.8, 121.3, 118.7, 118.6, 115.2, 114.4, 111.8, 109.7, 106.2, 36.6, 26.2, 24.7. ESI (m/z) 297 (MH+), 319 (MNa+).

### <Example 1-75> Preparation of N-(4-fluorophenyl)-4-(1H-indol-3-yl)butanamide

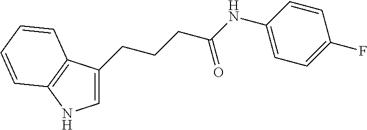 [SMILES: O=C(CCCc1c[nH]c2ccccc12)Nc1ccc(F)cc1]

The target compound was obtained by reacting in the same manner as in Examples 1-3.

1H NMR (400 MHz, DMSO-d6) δ 10.75 (s, 1H), 9.90 (s, 1H), 7.60 (d, 1H, J=8.8 Hz), 7.59 (d, 1H, J=8.8 Hz), 7.50 (d, 1H, J=7.6 Hz), 7.32 (d, 1H, J=8.0 Hz), 7.13-7.07 (m, 3H), 7.04 (t, 1H, J=7.2 Hz), 6.94 (t, 1H, J=7.4 Hz), 2.72 (t, 2H, J=7.4 Hz), 2.34 (t, 2H, J=7.4 Hz), 1.99-1.92 (m, 2H). 13C NMR (400 MHz, DMSO-d6) δ 171.5, 159.4, 136.8, 136.2, 127.6, 122.7, 121.3, 121.2, 121.1, 118.7, 118.5, 115.7, 115.5, 114.4, 111.8, 36.5, 26.3, 24.7. ESI (m/z) 297 (MH+), 319 (MNa+).

### <Example 1-76> Preparation of N-(4-bromophenyl)-4-(1H-indol-3-yl)butanamide

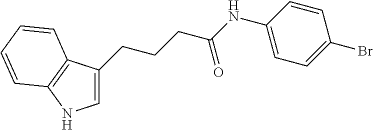 [SMILES: O=C(CCCc1c[nH]c2ccccc12)Nc1ccc(Br)cc1]

The target compound was obtained by reacting in the same manner as in Examples 1-3.

1H NMR (400 MHz, DMSO-d6) δ 10.75 (s, 1H), 9.98 (s, 1H), 7.56 (d, 2H, J=8.4 Hz), 7.49 (d, 1H, J=8.0 Hz), 7.43 (d, 2H, J=8.4 Hz), 7.31 (d, 1H, J=8.0 Hz), 7.10 (s, 1H), 7.03 (t, 1H, J=7.4 Hz), 6.94 (t, 1H, J=7.2 Hz), 2.71 (t, 2H, J=7.4 Hz), 2.35 (t, 2H, J=6.6 Hz), 1.99-1.93 (m, 2H). 13C NMR (400 MHz, DMSO-d6) δ 171.8, 139.2, 136.8, 131.9, 131.9, 127.6, 122.7, 121.4, 121.3, 121.3, 118.7, 118.6, 114.8, 114.4, 111.8, 36.6, 26.2, 24.7. ESI (m/z) 357 (MH+), 359 (MH+), 379 (MNa−), 381 (MNa−).

### <Example 1-77> Preparation of N-(3,5-bis(trifluoromethyl)phenyl)-4-(1H-indol-3-yl)butanamide

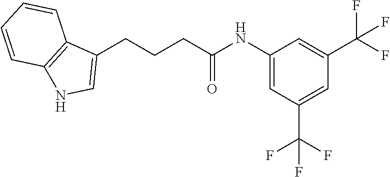 [SMILES: O=C(CCCc1c[nH]c2ccccc12)Nc1cc(C(F)(F)F)cc(C(F)(F)F)c1]

The target compound was obtained by reacting in the same manner as in Examples 1-3.

1H NMR (400 MHz, DMSO-d6) δ 10.76 (s, 1H), 10.51 (s, 1H), 8.25 (s, 1H), 7.69 (s, 2H), 7.50 (d, 1H, J=7.8 Hz), 7.32 (d, 1H, J=8.1 Hz), 7.12 (d, 2H, J=1.9 Hz), 7.04 (t, 1H, J=7.5 Hz), 6.94 (t, 1H, J=7.4 Hz), 2.74 (t, 2H, J=7.4 Hz), 2.41 (t, 2H, J=7.4 Hz), 2.03-1.92 (m, 2H). ESI (m/z) 415 (MH+).

### <Example 1-78> Preparation of N-(4-chloro-3-nitrophenyl)-4-(1H-indol-3-yl)butanamide

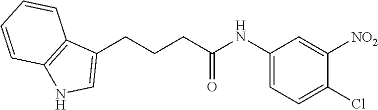 [SMILES: O=C(CCCc1c[nH]c2ccccc12)Nc1ccc(Cl)c([N+](=O)[O-])c1]

The target compound was obtained by reacting in the same manner as in Examples 1-3.

1H NMR (400 MHz, DMSO-d6) δ 10.75 (s, 1H), 10.40 (s, 1H), 8.39 (d, 1H, J=2.4 Hz), 7.73 (dd, 1H, J=8.9, 2.4 Hz), 7.64 (d, 1H, J=8.8 Hz), 7.49 (d, 1H, J=7.8 Hz), 7.30 (d, 1H, J=8.1 Hz), 7.10 (d, 1H, J=1.5 Hz), 7.02 (t, 1H, J=7.5 Hz), 6.93 (t, 1H, J=7.4 Hz), 2.71 (t, 2H, J=7.4 Hz), 2.38 (t, 2H, J=7.4 Hz), 2.01-1.90 (m, 2H, J=7.4 Hz). 13C NMR (400 MHz, DMSO-d6) δ 172.5, 147.6, 139.5, 136.7, 132.3, 127.5, 124.2, 122.8, 121.2, 118.7, 118.5, 118.4, 115.6, 114.2, 111.7, 36.5, 25.9, 24.6. ESI (m/z) 380 (MNa+), 356 (MH−).

### <Example 1-79> Preparation of N-(3,4-dichlorophenyl)-4-(1H-indol-3-yl)butanamide

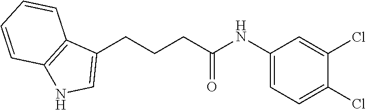 [SMILES: O=C(CCCc1c[nH]c2ccccc12)Nc1ccc(Cl)c(Cl)c1]

The target compound was obtained by reacting in the same manner as in Examples 1-3.

1H NMR (400 MHz, DMSO-d6) δ 10.75 (s, 1H), 10.14 (s, 1H), 7.98 (s, 1H), 7.55-7.41 (m, 3H), 7.30 (d, 1H, J=8.0 Hz), 7.10 (s, 1H), 7.03 (t, 1H, J=7.5 Hz), 6.93 (t, 1H, J=7.4 Hz), 2.70 (t, 2H, J=7.3 Hz), 2.35 (t, 2H, J=7.4 Hz), 1.98-1.90 (m, 2H). ESI (m/z) 369 (MNa+), 371 (MNa+), 345 (MH−).

### <Example 1-80> Preparation of methyl 4-((4-(1H-indol-3-yl)butanamido)methyl)benzoate

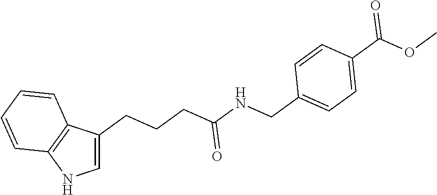 [SMILES: COC(=O)c1ccc(CNC(=O)CCCc2c[nH]c3ccccc23)cc1]

The target compound was obtained by reacting in the same manner as in Examples 1-3.

1H NMR (400 MHz, DMSO-d6) δ 10.77 (s, 1H), 8.43 (t, 1H, J=5.9 Hz), 7.91 (d, 2H, J=8.2 Hz), 7.48 (d, 1H, J=7.8 Hz), 7.38 (d, 2H, J=8.2 Hz), 7.33 (d, 1H, J=8.1 Hz), 7.10 (d, 1H, J=2.0 Hz), 7.05 (t, 1H, J=7.2 Hz), 6.95 (t, 1H, J=7.4 Hz), 4.34 (d, 2H, J=6.0 Hz), 3.82 (s, 3H), 2.68 (t, 2H, J=7.5 Hz), 2.24 (t, 2H, J=7.4 Hz), 1.96-1.87 (m, 2H). 13C NMR (400 MHz, DMSO-d6) δ 172.7, 166.5, 145.9, 136.7, 129.6, 129.6, 128.4, 127.7, 127.7, 127.6, 122.6, 121.2, 118.7, 118.5, 114.5, 111.7, 52.4, 42.2, 35.6, 26.6, 24.7. ESI (m/z) 351 (MH+), 373 (MNa+), 349 (NM−).

### <Example 1-81> Preparation of 4-(1H-indol-3-yl)-N-(4-methoxybenzyl)butanamide

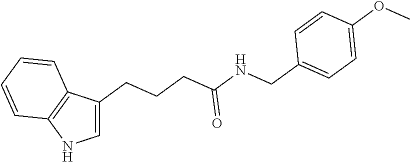 [SMILES: COc1ccc(CNC(=O)CCCc2c[nH]c3ccccc23)cc1]

The target compound was obtained by reacting in the same manner as in Examples 1-3.

1H NMR (400 MHz, DMSO-d6) δ 10.76 (s, 1H), 8.25 (t, 1H, J=5.8 Hz), 7.48 (d, 1H, J=7.8 Hz), 7.33 (d, 1H, J=8.1 Hz), 7.17 (d, 2H, J=8.5 Hz), 7.09 (d, 1H, J=1.9 Hz), 7.05 (t, 1H, J=7.5 Hz), 6.95 (t, 1H, J=7.4 Hz), 6.86 (d, 2H, J=8.6 Hz), 4.20 (d, 2H, J=5.9 Hz), 3.70 (s, 3H), 2.67 (t, 2H, J=7.5 Hz), 2.20 (t, 2H, J=7.4 Hz), 1.94-1.85 (m, 2H). 13C NMR (400 MHz, DMSO-d6) δ 172.4, 158.5, 136.7, 132.1, 128.9, 128.9, 127.6, 122.6, 121.2, 118.7, 118.5, 114.5, 114.0, 114.0, 111.7, 55.4, 41.9, 35.7, 26.7, 24.7. ESI (m/z) 323 (MH+), 345 (MNa+).

### <Example 1-82> Preparation of N-(3,4-dichlorobenzyl)-4-(1H-indol-3-yl)butanamide

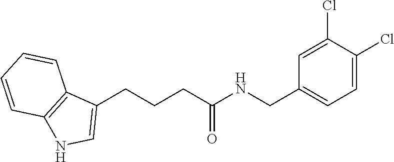 [SMILES: O=C(CCCc1c[nH]c2ccccc12)NCc1ccc(Cl)c(Cl)c1]

The target compound was obtained by reacting in the same manner as in Examples 1-3.

1H NMR (400 MHz, DMSO-d6) δ 10.74 (s, 1H), 8.38 (t, 1H, J=5.8 Hz), 7.53 (d, 1H, J=8.3 Hz), 7.48-7.41 (m, 2H), 7.30 (d, 1H, J=8.0 Hz), 7.21 (d, 1H, J=8.2 Hz), 7.07 (s, 1H), 7.03 (t, 1H, J=7.5 Hz), 6.93 (t, 1H, J=7.4 Hz), 4.23 (d, 2H, J=5.9 Hz), 2.65 (t, 2H, J=7.4 Hz), 2.20 (t, 2H, J=7.4 Hz), 1.93-1.81 (m, 2H). 13C NMR (400 MHz, DMSO-d6) δ 172.8, 141.5, 136.7, 131.2, 130.8, 129.6, 129.5, 127.9, 127.5, 122.6, 121.2, 118.7, 118.5, 114.4, 111.7, 41.4, 35.5, 26.5, 24.7. ESI (m/z) 383 (MNa+), 359 (MH−).

### <Example 1-83> Preparation of N-(4-fluorophenyl)-1H-indole-2-carboxamide

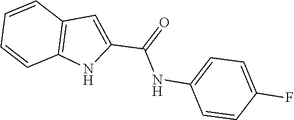 [SMILES: O=C(Nc1ccc(F)cc1)c1cc2ccccc2[nH]1]

The target compound was obtained by reacting in the same manner as in Examples 1-3.

1H NMR (400 MHz, DMSO-d6) δ 11.94 (s, 1H), 10.25 (s, 1H), 7.82 (d, 1H, J=8.4 Hz), 7.81 (d, 1H, J=8.8 Hz), 7.65 (d, 1H, J=8.0 Hz), 7.46 (d, 1H, J=8.0 Hz), 7.40 (s, 1H), 7.20 (d, 2H, J=8.8 Hz), 7.18 (t, 1H, J=5.6 Hz), 7.04 (t, 1H, J=7.4 Hz). 13C NMR (400 MHz, DMSO-d6) δ 160.1, 159.9, 137.3, 135.7, 131.8, 127.5, 124.2, 122.4, 122.3, 122.2, 120.4, 115.8, 115.6, 112.8, 104.3. ESI (m/z) 255 (MH+).

### <Example 1-84> Preparation of N-(3,5-bis(trifluoromethyl)phenyl)-1H-indole-2-carboxamide

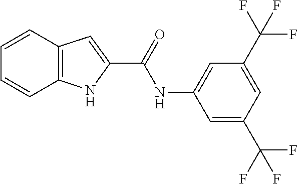 [SMILES: O=C(Nc1cc(C(F)(F)F)cc(C(F)(F)F)c1)c1cc2ccccc2[nH]1]

The target compound was obtained by reacting in the same manner as in Examples 1-3.

1H NMR (400 MHz, DMSO-d6) δ 11.83 (s, 1H), 10.77 (s, 1H), 8.53 (s, 2H), 7.77 (s, 1H), 7.70 (d, J=8.0 Hz, 1H), 7.48 (d, 1H, J=8.4 Hz), 7.47 (s, 1H), 7.24 (t, 1H, J=7.5 Hz), 7.08 (t, 1H, J=7.5 Hz). ESI (m/z) 373 (MH+), 371 (MH−).

### <Example 1-85> Preparation of N-(4-chloro-3-nitrophenyl)-1H-indole-2-carboxamide

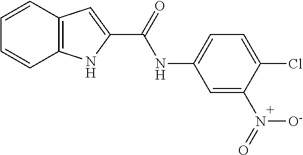

The target compound was obtained by reacting in the same manner as in Examples 1-3.

1H NMR (400 MHz, DMSO-d6) δ 11.81 (s, 1H), 10.67 (s, 1H), 8.58 (d, 1H, J=2.4 Hz), 8.08 (dd, 1H, J=8.9, 2.5 Hz), 7.73 (d, 1H, J=8.9 Hz), 7.67 (d, 1H, J=8.0 Hz), 7.48-7.42 (m, 2H), 7.22 (t, 1H, J=7.5 Hz), 7.05 (t, 1H, J=7.5 Hz). 13C NMR (400 MHz, DMSO-d6) δ 160.5, 147.5, 139.4, 137.5, 132.4, 130.9, 127.3, 125.1, 124.7, 122.4, 120.5, 119.0, 116.6, 112.9, 105.2. ESI (m/z) 314 (MH−).

### <Example 1-86> Preparation of N-(3,4-dichlorophenyl)-1H-indole-2-carboxamide

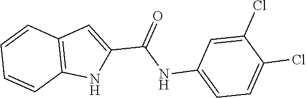 [SMILES: O=C(Nc1ccc(Cl)c(Cl)c1)c1cc2ccccc2[nH]1]

The target compound was obtained by reacting in the same manner as in Examples 1-3.

1H NMR (400 MHz, DMSO-d6) δ 11.78 (s, 1H), 10.42 (s, 1H), 8.15 (d, 1H, J=2.4 Hz), 7.77 (dd, 1H, J=8.8, 2.4 Hz), 7.66 (d, 1H, J=8.0 Hz), 7.60 (t, 1H, J=9.4 Hz), 7.45 (d, 1H, J=8.2 Hz), 7.41 (s, 1H), 7.21 (t, 1H, J=7.6 Hz), 7.05 (t, 1H, J=7.5 Hz). 13C NMR (400 MHz, DMSO-d6) δ 160.3, 139.5, 137.4, 131.3, 131.2, 131.0, 127.3, 125.3, 124.5, 122.3, 121.5, 120.4, 120.3, 112.8, 104.9. ESI (m/z) 303 (MH−).

### <Example 1-87> Preparation of N-(3,4-dimethoxyphenyl)-1H-indole-2-carboxamide

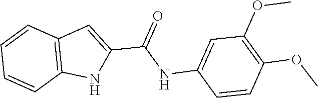 [SMILES: COc1ccc(NC(=O)c2cc3ccccc3[nH]2)cc1OC]

The target compound was obtained by reacting in the same manner as in Examples 1-3. ESI (m/z) 295 (MH−).

### <Example 1-88> Preparation of methyl 4-((1H-indole-2-carboxamido)methyl)benzoate

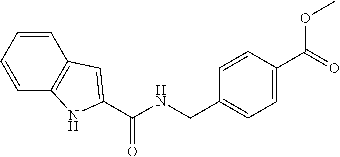 [SMILES: COC(=O)c1ccc(CNC(=O)c2cc3ccccc3[nH]2)cc1]

The target compound was obtained by reacting in the same manner as in Examples 1-3.

1H NMR (400 MHz, DMSO-d6) δ 11.63 (s, 1H), 9.13 (t, 1H, J=6.1 Hz), 7.93 (d, 2H, J=8.3 Hz), 7.61 (d, 1H, J=8.0 Hz), 7.47 (d, 2H, J=8.2 Hz), 7.43 (d, 1H, J=8.2 Hz), 7.22-7.13 (m, 2H), 7.03 (t, 1H, J=7.4 Hz), 4.59 (d, 2H, J=6.0 Hz), 3.82 (s, 3H). 13C NMR (400 MHz, DMSO-d6) δ 166.5, 161.7, 145.7, 136.9, 131.8, 129.7, 129.7, 128.6, 127.7, 127.5, 127.5, 123.8, 121.9, 120.2, 112.7, 103.1, 52.4, 42.4. ESI (m/z) 307 (MH−).

### <Example 1-89> Preparation of N′-(3,4-dichlorophenyl)-1H-indole-2-carbohydrazide

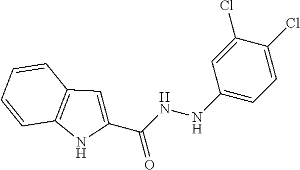 [SMILES: O=C(NNc1ccc(Cl)c(Cl)c1)c1cc2ccccc2[nH]1]

The target compound was obtained by reacting in the same manner as in Examples 1-3.

1H NMR (400 MHz, DMSO-d6) δ 11.70 (s, 1H), 10.46 (s, 1H), 8.41 (s, 1H), 7.62 (d, 1H, J=8.0 Hz), 7.42 (d, 1H, J=8.2 Hz), 7.35 (d, 1H, J=8.8 Hz), 7.26 (s, 1H), 7.18 (t, 1H, J=7.5 Hz), 7.03 (t, 1H, J=7.4 Hz), 6.90 (s, 1H), 6.75 (d, 1H, J=8.8 Hz). 13C NMR (400 MHz, DMSO-d6) δ 161.7, 150.1, 137.1, 131.7, 131.1, 129.8, 127.4, 124.1, 122.1, 120.4, 119.8, 113.4, 112.9, 112.8, 103.7. ESI (m/z) 342 (MNa+), 318 (MH−).

### <Example 1-90> Preparation of 5-fluoro-N-(4-fluorophenyl)-1H-indole-2-carboxamide

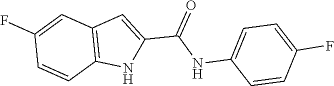 [SMILES: O=C(Nc1ccc(F)cc1)c1cc2cc(F)ccc2[nH]1]

The target compound was obtained by reacting in the same manner as in Examples 1-3.

1H NMR (400 MHz, DMSO-d6) δ 10.92 (s, 1H), 10.57 (s, 1H), 8.62 (s, 1H), 7.92 (d, 1H, J=8.0 Hz), 7.85 (d, 1H, J=8.0 Hz), 7.58 (d, 1H, J=8.8 Hz), 7.55 (t, 1H, J=8.4 Hz), 7.34 (d, 1H, J=8.0 Hz), 7.27 (s, 1H), 7.05 (t, 1H, J=7.2 Hz), 6.97 (t, 1H, J=7.2 Hz), 3.77 (s, 2H).

### <Example 1-91> Preparation of N-(3,5-bis(trifluoromethyl)phenyl)-5-fluoro-1H-indole-2-carboxamide

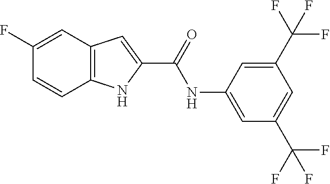 [SMILES: O=C(Nc1cc(C(F)(F)F)cc(C(F)(F)F)c1)c1cc2cc(F)ccc2[nH]1]

The target compound was obtained by reacting in the same manner as in Examples 1-3.

1H NMR (400 MHz, DMSO-d6) δ 11.93 (s, 1H), 10.79 (s, 1H), 8.50 (s, 2H), 7.78 (s, 1H), 7.51-7.41 (m, 3H), 7.09 (td, 1H, J=9.3, 2.5 Hz). ESI (m/z) 389 (MH−).

### <Example 1-92> Preparation of N-(3,5-difluorophenyl)-5-fluoro-1H-indole-2-carboxamide

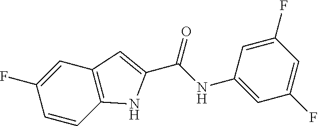 [SMILES: O=C(Nc1cc(F)cc(F)c1)c1cc2cc(F)ccc2[nH]1]

The target compound was obtained by reacting in the same manner as in Examples 1-3.

1H NMR (400 MHz, DMSO-d6) δ 11.91 (s, 1H), 10.53 (s, 1H), 7.58-7.51 (m, 2H), 7.47 (dd, 1H, J=10.0, 2.5 Hz), 7.45-7.41 (m, 1H), 7.39 (s, 1H), 7.08 (td, 1H, J=9.2, 2.3 Hz), 6.94 (t, 1H, J=9.3 Hz). ESI (m/z) 289 (MH−).

### <Example 1-93> Preparation of N-(3,4-dimethoxyphenyl)-5-fluoro-1H-indole-2-carboxamide

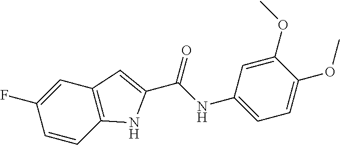 [SMILES: COc1ccc(NC(=O)c2cc3cc(F)ccc3[nH]2)cc1OC]

The target compound was obtained by reacting in the same manner as in Examples 1-3.

1H NMR (400 MHz, DMSO-d6) δ 11.79 (s, 1H), 10.10 (s, 1H), 7.46-7.39 (m, 3H), 7.37-7.32 (m, 2H), 7.05 (td, 1H, J=9.3, 2.4 Hz), 6.92 (d, 1H, J=8.7 Hz), 3.74 (s, 3H), 3.71 (s, 3H). ESI (m/z) 315 (MH+), 337 (MNa+), 313 (MH−).

### <Example 1-94> Preparation of N-(3,4-dimethoxybenzyl)-5-fluoro-1H-indole-2-carboxamide

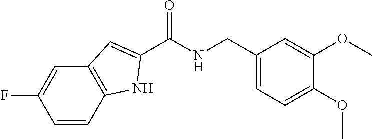 [SMILES: COc1ccc(CNC(=O)c2cc3cc(F)ccc3[nH]2)cc1OC]

The target compound was obtained by reacting in the same manner as in Examples 1-3.

1H NMR (400 MHz, DMSO-d6) δ 11.68 (s, 1H), 8.97 (t, 1H, J=5.9 Hz), 7.41-7.34 (m, 2H), 7.13 (s, 1H), 7.00 (td, 1H, J=9.3, 2.4 Hz), 6.93 (s, 1H), 6.89-6.80 (m, 2H), 4.40 (d, 2H, J=5.9 Hz), 3.70 (s, 3H), 3.69 (s, 3H). ESI (m/z) 327 (MH−).

### <Example 1-95> Preparation of 5-fluoro-N-(3-(trifluoromethyl)phenyl)-1H-indole-2-carboxamide

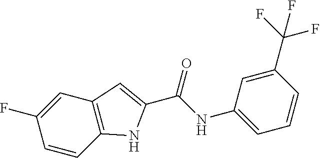 [SMILES: O=C(Nc1cccc(C(F)(F)F)c1)c1cc2cc(F)ccc2[nH]1]

The target compound was obtained by reacting in the same manner as in Examples 1-3.

1H NMR (400 MHz, DMSO-d6) δ 11.92 (s, 1H), 10.56 (s, 1H), 8.25 (s, 1H), 8.12 (d, 1H, J=8.3 Hz), 7.63 (t, 1H, J=8.0 Hz), 7.54-7.44 (m, 4H), 7.12 (td, 1H, J=9.4, 2.5 Hz). ESI (m/z) 323 (MH+), 321 (MH−).

### <Example 1-96> Preparation of N-(3,5-dimethoxyphenyl)-5-fluoro-1H-indole-2-carboxamide

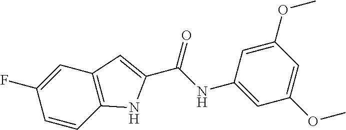 [SMILES: COc1cc(NC(=O)c2cc3cc(F)ccc3[nH]2)cc(OC)c1]

The target compound was obtained by reacting in the same manner as in Examples 1-3.

1H NMR (400 MHz, DMSO-d6) δ 11.86 (s, 1H), 10.16 (s, 1H), 7.50-7.43 (m, 2H), 7.41 (d, 1H, J=1.5 Hz), 7.13-7.06 (m, 3H), 6.29 (t, 1H, J=2.2 Hz), 3.76 (s, 6H). ESI (m/z) 315 (MH+), 313 (MH−).

### <Example 1-97> Preparation of 5-fluoro-N-(2-fluorophenyl)-1H-indole-2-carboxamide

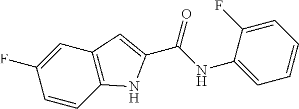 [SMILES: O=C(Nc1ccccc1F)c1cc2cc(F)ccc2[nH]1]

The target compound was obtained by reacting in the same manner as in Examples 1-3.

1H NMR (400 MHz, DMSO-d6) δ 11.89 (s, 1H), 10.16 (s, 1H), 7.64 (td, 1H, J=7.8, 1.7 Hz), 7.49-7.43 (m, 2H), 7.39 (d, 1H, J=1.5 Hz), 7.35-7.22 (m, 3H), 7.10 (td, 1H, J=9.3, 2.5 Hz). ESI (m/z) 273 (MH+), 271 (MH−).

### <Example 1-98> Preparation of 5-fluoro-N-(3-fluorophenyl)-1H-indole-2-carboxamide

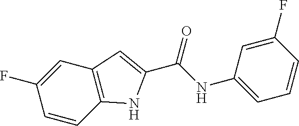 [SMILES: O=C(Nc1cccc(F)c1)c1cc2cc(F)ccc2[nH]1]

The target compound was obtained by reacting in the same manner as in Examples 1-3.

1H NMR (400 MHz, DMSO-d6) δ 11.91 (s, 1H), 10.43 (s, 1H), 7.79 (dt, 1H, J=11.8, 2.1 Hz), 7.59 (dd, J=8.2, 0.9 Hz, 1H), 7.52-7.38 (m, 4H), 7.11 (td, J=9.3, 2.5 Hz, 1H), 6.95 (td, J=8.4, 2.0 Hz, 1H). ESI (m/z) 273 (MH+), 271 (MH−).

### <Example 1-99> Preparation of N-(3,5-bis(trifluoromethyl)phenyl)-5-chloro-1H-indole-2-carboxamide

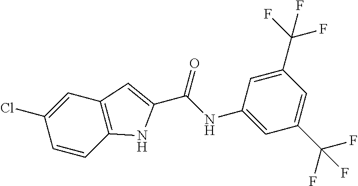 [SMILES: O=C(Nc1cc(C(F)(F)F)cc(C(F)(F)F)c1)c1cc2cc(Cl)ccc2[nH]1]

The target compound was obtained by reacting in the same manner as in Examples 1-3.

1H NMR (400 MHz, DMSO-d6) δ 12.07 (s, 1H), 10.87 (s, 1H), 8.54 (s, 2H), 7.84 (d, 2H, J=7.6 Hz), 7.52-7.45 (m, 2H), 7.27 (d, 1H, J=8.8 Hz). ESI (m/z) 407 (MH+), 405 (MH−).

### <Example 1-100> Preparation of methyl 2-((3,5-bis(trifluoromethyl)phenyl)carbamoyl)-1H-indole-5-carboxylate

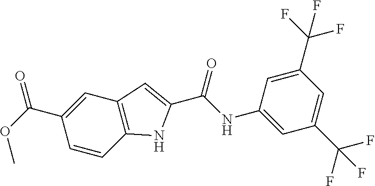 [SMILES: COC(=O)c1ccc2[nH]c(C(=O)Nc3cc(C(F)(F)F)cc(C(F)(F)F)c3)cc2c1]

The target compound was obtained by reacting in the same manner as in Examples 1-3.

1H NMR (400 MHz, DMSO-d6) δ 12.21 (s, 1H), 10.87 (s, 1H), 8.49 (s, 2H), 8.40 (s, 1H), 7.82 (d, J=8.7 Hz, 1H), 7.76 (s, 1H), 7.57 (s, 1H), 7.52 (d, J=8.7 Hz, 1H), 3.82 (s, 3H). ESI (m/z) 429 (MH−).

### <Example 1-101> Preparation of 2-((3,5-bis(trifluoromethyl)phenyl)carbamoyl)-1H-indole-5-carboxylic acid

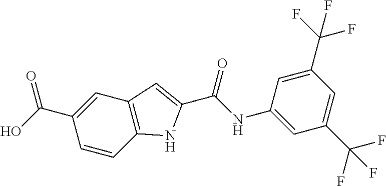 [SMILES: O=C(O)c1ccc2[nH]c(C(=O)Nc3cc(C(F)(F)F)cc(C(F)(F)F)c3)cc2c1]

The target compound was obtained by reacting in the same manner as in Example 1-3.

1H NMR (400 MHz, DMSO-d6) δ 12.63 (s, 1H), 12.22 (s, 1H), 10.93 (s, 1H), 8.55 (s, 2H), 8.43 (s, 1H), 7.89-7.81 (m, 2H), 7.62 (s, 1H), 7.55 (d, 1H, J=8.7 Hz). ESI (m/z) 415 (MH−).

### <Example 1-102> Preparation of N-(3,5-bis(trifluoromethyl)phenyl)-5-(trifluoromethyl)-1H-indole-2-carboxamide

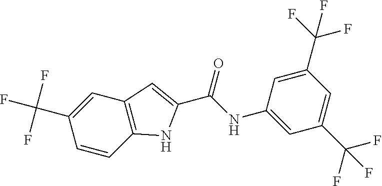 [SMILES: O=C(Nc1cc(C(F)(F)F)cc(C(F)(F)F)c1)c1cc2cc(C(F)(F)F)ccc2[nH]1]

The target compound was obtained by reacting in the same manner as in Example 1-3.

1H NMR (400 MHz, DMSO-d6) δ 12.32 (s, 1H), 10.97 (s, 1H), 8.55 (s, 2H), 8.23 (s, 1H), 7.84 (s, 1H), 7.70-7.63 (m, 2H), 7.55 (d, J=8.6 Hz, 1H). ESI (m/z) 439 (MH−).

### <Example 1-103> Preparation of N-(3,5-dimethylphenyl)-5-fluoro-1H-indole-2-carboxamide

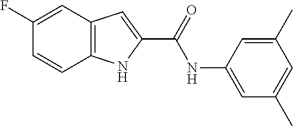 [SMILES: Cc1cc(C)cc(NC(=O)c2cc3cc(F)ccc3[nH]2)c1]

The target compound was obtained by reacting in the same manner as in Example 1-3.

1H NMR (400 MHz, DMSO-d6) δ 11.75 (s, 1H), 10.05 (s, 1H), 7.45-7.35 (m, 5H), 7.05 (t, J=9.2 Hz, 1H), 6.72 (s, 1H), 2.24 (s, 6H). ESI (m/z) 281 (MH−).

### <Example 1-104> Preparation of N-(3,5-dimethylphenyl)-1H-indole-2-carboxamide

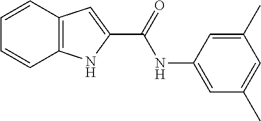 [SMILES: Cc1cc(C)cc(NC(=O)c2cc3ccccc3[nH]2)c1]

The target compound was obtained by reacting in the same manner as in Example 1-3.

1H NMR (400 MHz, DMSO-d6) δ 11.64 (s, 1H), 10.00 (s, 1H), 7.63 (d, J=8.0 Hz, 1H), 7.46-7.35 (m, 4H), 7.18 (t, J=7.6 Hz, 1H), 7.03 (t, J=7.5 Hz, 1H), 6.72 (s, 1H), 2.24 (s, 6H).

### <Example 1-105> Preparation of N-(3,4-dihydroxyphenyl)-1H-indole-3-carboxamide

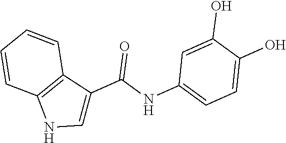 [SMILES: O=C(Nc1ccc(O)c(O)c1)c1c[nH]c2ccccc12]

The target compound was obtained by reacting in the same manner as in Example 1-3.

1H NMR (400 MHz, DMSO-d6) δ 11.60 (s, 1H), 9.35 (s, 1H), 8.86 (s, 1H), 8.50 (s, 1H), 8.18 (d, 1H, J=2.8 Hz), 8.14 (d, 1H, J=7.6 Hz), 7.41 (d, 1H, J=7.9 Hz), 7.28 (d, 1H, J=2.4 Hz), 7.16-7.06 (m, 2H), 6.90 (dd, 1H, J=8.5, 2.4 Hz), 6.63 (d, 1H, J=8.5 Hz). ESI (m/z) 267 (MH−).

### <Example 1-106> Preparation of N-(3,4-dihydroxyphenyl)-2-(1H-indol-3-yl)acetamide

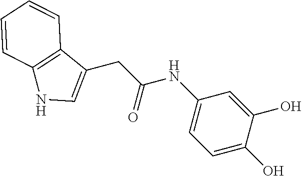 [SMILES: O=C(Cc1c[nH]c2ccccc12)Nc1ccc(O)c(O)c1]

The target compound was obtained by reacting in the same manner as in Example 1-3.

1H NMR (400 MHz, DMSO-d6) δ 10.85 (s, 1H), 9.69 (s, 1H), 8.89 (s, 1H), 8.53 (s, 1H), 7.58 (d, 1H, J=7.9 Hz), 7.32 (d, 1H, J=8.0 Hz), 7.20 (s, 1H), 7.13 (s, 1H), 7.04 (t, 1H, J=7.5 Hz), 6.95 (t, 1H, J=7.4 Hz), 6.76 (d, 1H, J=8.5 Hz), 6.59 (d, 1H, J=8.4 Hz), 3.63 (s, 2H). 13C NMR (400 MHz, DMSO-d6) δ 169.3, 145.3, 141.5, 136.5, 131.9, 127.6, 124.1, 121.3, 119.1, 118.7, 115.6, 111.7, 110.7, 109.3, 108.3, 34.1. ESI (m/z) 281 (MH−).

### <Example 1-107> Preparation of N-(3,4-dihydroxybenzyl)-2-(1H-indol-3-yl)acetamide

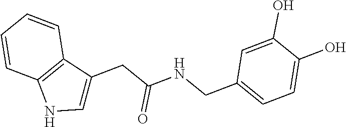 [SMILES: O=C(Cc1c[nH]c2ccccc12)NCc1ccc(O)c(O)c1]

The target compound was obtained by reacting in the same manner as in Example 1-3. 13C NMR (400 MHz, DMSO-d6) δ 170.8, 145.4, 144.5, 136.4, 130.7, 127.6, 124.1, 121.3, 119.1, 118.7, 118.6, 115.6, 115.5, 111.6, 109.3, 42.4, 33.0. ESI (m/z) 319 (MNa+).

### <Example 1-108> Preparation of N-(3,4-dihydroxyphenyl)-3-(1H-indol-3-yl)propanamide

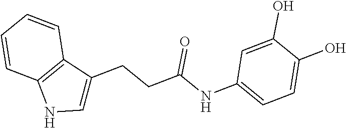 [SMILES: O=C(CCc1c[nH]c2ccccc12)Nc1ccc(O)c(O)c1]

The target compound was obtained by reacting in the same manner as in Example 1-3.

1H NMR (400 MHz, DMSO-d6) δ 10.72 (s, 1H), 9.51 (s, 1H), 8.87 (s, 1H), 8.50 (s, 1H), 7.53 (d, 1H, J=7.8 Hz), 7.29 (d, 1H, J=8.0 Hz), 7.12 (d, 1H, J=2.2 Hz), 7.08 (s, 1H), 7.03 (t, 1H, J=7.5 Hz), 6.94 (t, 1H, J=7.4 Hz), 6.74 (dd, 1H, J=8.4, 2.2 Hz), 6.57 (d, 1H, J=8.5 Hz), 2.95 (t, 2H, J=7.6 Hz), 2.57 (t, 2H, J=7.6 Hz). ESI (m/z) 295 (MNa+).

### <Example 1-109> Preparation of N-(3,4-dihydroxyphenyl)-1H-indole-2-carboxamide

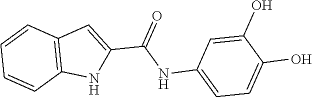 [SMILES: O=C(Nc1ccc(O)c(O)c1)c1cc2ccccc2[nH]1]

The target compound was obtained by reacting in the same manner as in Example 1-3.

1H NMR (400 MHz, DMSO-d6) δ 11.62 (s, 1H), 9.87 (s, 1H), 8.84 (s, 2H), 7.61 (d, 1H, J=8.0 Hz), 7.42 (d, 1H, J=8.2 Hz), 7.34-7.27 (m, 2H), 7.16 (t, 1H, J=7.6 Hz), 7.05-6.95 (m, 2H), 6.68 (d, 1H, J=8.5 Hz). 13C NMR (400 MHz, DMSO-d6) δ 159.5, 145.3, 142.1, 137.0, 132.3, 131.2, 127.5, 123.9, 122.0, 120.2, 115.6, 112.7, 112.0, 109.3, 103.6. ESI (m/z) 267 (MH−).

### <Example 1-110> Preparation of N-(3,4-dihydroxyphenyl)-5-fluoro-1H-indole-2-carboxamide

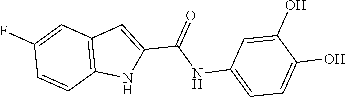 [SMILES: O=C(Nc1ccc(O)c(O)c1)c1cc2cc(F)ccc2[nH]1]

The target compound was obtained by reacting in the same manner as in Example 1-3.

1H NMR (400 MHz, DMSO-d6) δ 11.73 (s, 1H), 9.91 (s, 1H), 8.99 (s, 1H), 8.69 (s, 1H), 7.46-7.36 (m, 2H), 7.30 (d, 2H, J=12.3 Hz), 7.03 (td, 1H, J=9.3, 1.9 Hz), 6.98 (dd, 1H, J=8.5, 1.7 Hz), 6.68 (d, 1H, J=8.5 Hz). ESI (m/z) 285 (MH−).

### <Example 1-111> Preparation of N-(3,4-dihydroxybenzyl)-5-fluoro-1H-indole-2-carboxamide

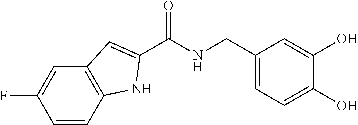 [SMILES: O=C(NCc1ccc(O)c(O)c1)c1cc2cc(F)ccc2[nH]1]

The target compound was obtained by reacting in the same manner as in Example 1-3.

1H NMR (400 MHz, DMSO-d6) δ 11.66 (s, 1H), 8.90 (t, 1H, J=6.0 Hz), 8.82 (s, 1H), 8.70 (s, 1H), 7.41-7.33 (m, 2H), 7.11 (s, 1H), 7.00 (td, 1H, J=9.2, 2.3 Hz), 6.69 (d, 1H, J=1.3 Hz), 6.64 (d, 1H, J=8.0 Hz), 6.55 (d, 1H, J=8.0 Hz), 4.30 (d, 2H, J=5.9 Hz). ESI (m/z) 301 (MH+).

### <Example 1-112> Preparation of N-(3,5-dihydroxyphenyl)-5-fluoro-1H-indole-2-carboxamide

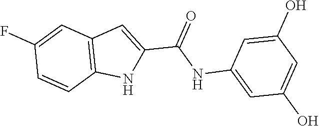 [SMILES: O=C(Nc1cc(O)cc(O)c1)c1cc2cc(F)ccc2[nH]1]

The target compound was obtained by reacting in the same manner as in Examples 1-3.

1H NMR (400 MHz, DMSO-d6) δ 11.75 (s, 1H), 9.92 (s, 1H), 9.21 (s, 2H), 7.44-7.38 (m, 2H), 7.35 (s, 1H), 7.04 (td, 1H, J=9.3, 2.3 Hz), 6.74 (d, 2H, J=1.9 Hz), 5.94 (s, 1H). ESI (m/z) 285 (MH−).

### <Example 1-113> Preparation of N-(3,5-bis(trifluoromethyl)phenyl)-5,6-difluoro-1H-indole-2-carboxamide

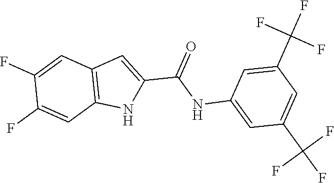 [SMILES: O=C(Nc1cc(C(F)(F)F)cc(C(F)(F)F)c1)c1cc2cc(F)c(F)cc2[nH]1]

The target compound was obtained by reacting in the same manner as in Examples 1-3.

1H NMR (400 MHz, DMSO-d6) δ 12.05 (s, 1H), 10.83 (s, 1H), 8.52 (s, 2H), 7.86-7.78 (m, 2H), 7.50 (s, 1H), 7.40 (dd, J=10.8, 7.1 Hz, 1H). ESI (m/z) 407 (MH−).

### <Example 1-114> Preparation of N-(3,5-bis(trifluoromethyl)phenyl)-5,6-difluoro-1-methyl-1H-indole-2-carboxamide

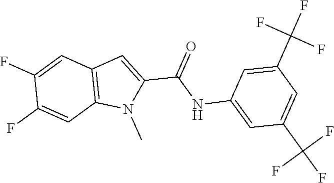 [SMILES: Cn1c(C(=O)Nc2cc(C(F)(F)F)cc(C(F)(F)F)c2)cc2cc(F)c(F)cc21]

The target compound was obtained by reacting in the same manner as in Examples 1-3.

1H NMR (400 MHz, DMSO-d6) δ 10.89 (s, 3H), 8.51 (s, 7H), 7.85-7.76 (m, 10H), 7.44 (s, 3H), 4.01 (s, 9H). ESI (m/z) 421 (MH−).

### <Example 1-115> Preparation of 4-nitrobenzyl 2-(1H-indol-3-yl)acetate

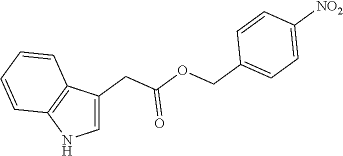 [SMILES: O=C(Cc1c[nH]c2ccccc12)OCc1ccc([N+](=O)[O-])cc1]

The target compound was obtained by reacting in the same manner as in Examples 1-3.

1H NMR (400 MHz, DMSO-d6) δ 10.94 (s, 1H), 8.14 (d, 2H, J=8.4 Hz), 7.54 (d, 2H, J=8.4 Hz), 7.48 (d, 1H, J=7.6 Hz), 7.34 (d, 1H, J=8.0 Hz), 7.26 (s, 1H), 7.06 (t, 1H, J=7.4 Hz), 6.96 (t, 1H, J=7.2 Hz), 5.22 (s, 2H), 3.84 (s, 2H). 13C NMR (400 MHz, DMSO-d6) δ 171.7, 147.4, 144.4, 136.5, 128.8, 128.8, 127.4, 124.6, 123.8, 123.8, 121.5, 118.9, 118.9, 111.9, 107.1, 64.8, 31.1. ESI (m/z) 309 (MH−).

### <Example 1-116> Preparation of 4-methoxybenzyl 1H-indole-2-carboxylate

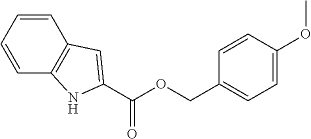 [SMILES: COc1ccc(COC(=O)c2cc3ccccc3[nH]2)cc1]

The target compound was obtained by reacting in the same manner as in Examples 1-3.

1H NMR (400 MHz, DMSO-d6) δ 11.91 (s, 1H), 7.63 (d, 1H, J=8.0 Hz), 7.46 (d, 1H, J=8.3 Hz), 7.42 (d, 2H, J=8.6 Hz), 7.28-7.21 (m, 1H), 7.16 (d, 1H, J=1.6 Hz), 7.05 (t, 1H, J=7.5 Hz), 6.97-6.92 (m, 2H), 5.30 (s, 2H), 3.74 (s, 3H). 13C NMR (400 MHz, DMSO-d6) δ 161.6, 159.6, 137.9, 130.4, 130.4, 128.4, 127.5, 127.1, 125.1, 122.5, 120.6, 114.3, 114.3, 113.0, 108.4, 66.1, 55.5.

### <Example 1-117> Preparation of 4-methoxybenzyl 4-(1H-indol-3-yl)butanoate

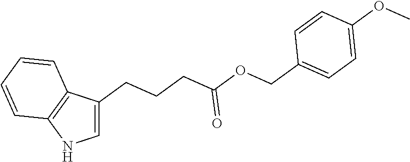 [SMILES: COc1ccc(COC(=O)CCCc2c[nH]c3ccccc23)cc1]

The target compound was obtained by reacting in the same manner as in Examples 1-3.

1H NMR (400 MHz, DMSO-d6) δ 10.76 (s, 1H), 7.43 (s, 1H), 7.32 (d, 1H, J=8.1 Hz), 7.28 (d, 2H, J=8.6 Hz), 7.07 (d, 1H, J=2.2 Hz), 7.03 (d, 1H, J=7.3 Hz), 6.93 (t, 1H, J=7.4 Hz), 6.90 (d, 2H, J=8.6 Hz), 5.00 (s, 2H), 3.72 (s, 3H), 2.67 (t, 2H, J=7.5 Hz), 2.35 (t, 2H, J=7.3 Hz), 1.93-1.83 (m, 2H). 13C NMR (400 MHz, DMSO-d6) δ 173.2, 159.5, 136.7, 130.4, 130.4, 128.6, 127.5, 122.8, 121.2, 118.6, 118.5, 114.2, 114.1, 114.1, 111.7, 65.6, 55.5, 33.7, 25.9, 24.3. ESI (m/z) 346 (MNa+).

### <Example 1-118> Preparation of 3,5-bis(trifluoromethyl)phenyl 5-fluoro-1H-indole-2-carboxylate

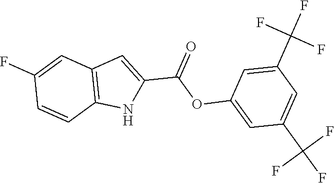 [SMILES: O=C(Oc1cc(C(F)(F)F)cc(C(F)(F)F)c1)c1cc2cc(F)ccc2[nH]1]

The target compound was obtained by reacting in the same manner as in Examples 1-3.

1H NMR (400 MHz, DMSO-d6) δ 12.33 (s, 1H), 8.20 (s, 2H), 8.08 (s, 1H), 7.52-7.45 (m, 2H), 7.41 (s, 1H), 7.18 (td, 1H, J=9.3, 2.5 Hz). ESI (m/z) 390 (MH−).

### <Example 1-119> Preparation of N-(3,5-bis(trifluoromethyl)phenyl)-5-(2-(5-((3aS,4S,6aR)-2-oxohexahydro-1H-thieno[3,4-d]imidazol-4-yl)pentanoyl)hydrazine-1-carbonyl)-1H-indole-2-carboxamide

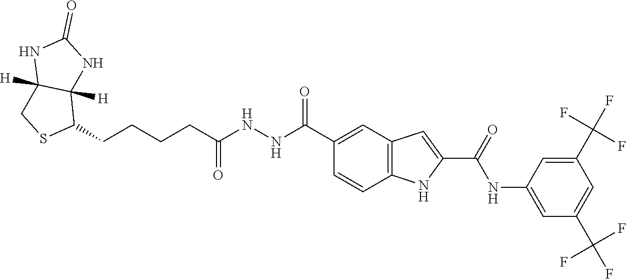

The target compound was obtained by reacting in the same manner as in Examples 1-3. ESI (m/z) 655 (MH−) 657 (MH+).

### Example 2. Evaluation of PLD Inhibitory Activity

To evaluate the inhibitory effect of the compounds according to the present invention on phospholipase D1 activity, experiments were conducted as follows.

For the analysis of PLD activity in cells, human colon cancer cell line (HCT116) was cultured in a 6-well plate at 37° C. for 24 hours, washed twice with phosphate-buffered saline (PBS), and then replaced with medium containing [³H]myristate and cultured for 24 hours. After the culture, the cells were washed three times with PBS, treated with 1-butanol, and the drug was added to a final concentration of 10 μM. The cells were trypsinized and suspended, and the amount of [³H]phosphatidylbutanol produced in the cell suspension was measured and evaluated for PLD activity using thin layer chromatography (TLC), and the results are shown in Table 1 below.

The Remaining activity % at 10 μM in Table 1 is calculated by setting the amount of [³H]phosphatidylbutanol produced in cells without the compound as 100% and calculating the amount of [³H]phosphatidylbutanol produced in cells treated with the compound as a percentage. Therefore, the lower the value of Remaining activity %, the higher the PLD inhibitory activity. Additionally, to confirm the reproducibility of the experiment, the known PLD1 inhibitor VU0155069 was treated at 10 μM in cells and PLD activity was measured in the same manner, and the value was shown next to each example value in the Remaining activity % at 10 μM.

TABLE 1

| | | Remaining |
|---|---|---|
| | Remaining | activity % at |
| | activity % at | 10 μM |
| Compound | 10 μM | (VU0155069) |
| Example 1-1 | 86.43 | 34.66 |
| Example 1-2 | 85.52 | 34.66 |
| Example 1-3 | 62.73 | 23.32 |
| Example 1-4 | 56.19 | 23.32 |
| Example 1-5 | 89.34 | 34.66 |
| Example 1-6 | 86.59 | 23.32 |
| Example 1-7 | 65.03 | 27.04 |
| Example 1-8 | 65.7 | 27.04 |
| Example 1-9 | 80.82 | 27.04 |
| Example 1-10 | 89.33 | 27.04 |
| Example 1-11 | not active | 27.04 |
| Example 1-12 | not active | 27.04 |
| Example 1-13 | 30.28 | 15.03 |
| Example 1-14 | not active | 27.04 |
| Example 1-15 | 30.42 | 15.03 |
| Example 1-16 | 97.25 | 27.04 |
| Example 1-17 | not active | 27.04 |
| Example 1-18 | 97.19 | 34.66 |
| Example 1-19 | not active | 34.66 |
| Example 1-20 | not active | 34.66 |
| Example 1-21 | 46.59 | 23.32 |
| Example 1-22 | 66.5 | 23.32 |
| Example 1-23 | not active | 27.04 |
| Example 1-24 | not active | 27.04 |
| Example 1-25 | 66.73 | 27.04 |
| Example 1-26 | 82.43 | 27.04 |
| Example 1-27 | 81.22 | 27.04 |
| Example 1-28 | not active | 34.66 |
| Example 1-29 | 76.8 | 23.51 |
| Example 1-30 | not active | 23.51 |
| Example 1-31 | not active | 23.51 |
| Example 1-32 | 40.95 | 23.51 |
| Example 1-33 | 83.43 | 23.51 |
| Example 1-34 | 66.07 | 23.51 |
| Example 1-35 | 83.61 | 27.04 |
| Example 1-36 | 76.23 | 27.04 |
| Example 1-37 | not active | 27.04 |
| Example 1-38 | 99.81 | 23.51 |
| Example 1-39 | 91.57 | 23.51 |
| Example 1-40 | 95.87 | 29.68 |
| Example 1-41 | 59.99 | 16.12 |
| Example 1-42 | 34.44 | 12.3 |
| Example 1-43 | 48.92 | 12.3 |
| Example 1-44 | 54.83 | 27.84 |
| Example 1-45 | 41.05 | 12.3 |
| Example 1-46 | 57.41 | 16.12 |
| Example 1-47 | 62.96 | 23.32 |
| Example 1-48 | 75.72 | 27.04 |
| Example 1-49 | 59.74 | 27.04 |
| Example 1-50 | 79.27 | 27.04 |
| Example 1-51 | 82.82 | 27.04 |
| Example 1-52 | 95.18 | 27.04 |
| Example 1-53 | not active | 27.04 |
| Example 1-54 | not active | 23.32 |
| Example 1-55 | 77.89 | 23.32 |
| Example 1-56 | 96.79 | 34.66 |
| Example 1-57 | not active | 34.66 |
| Example 1-58 | not active | 34.66 |
| Example 1-59 | not active | 27.04 |
| Example 1-60 | 82.15 | 29.68 |
| Example 1-61 | 45.47 | 23.51 |
| Example 1-62 | 49.36 | 29.68 |
| Example 1-63 | not active | 23.51 |
| Example 1-64 | not active | 23.51 |
| Example 1-65 | not active | 23.51 |
| Example 1-66 | 84.92 | 34.66 |
| Example 1-67 | 29.17 | 33.33 |
| Example 1-68 | not active | 34.66 |
| Example 1-69 | not active | 34.66 |
| Example 1-70 | not active | 34.66 |
| Example 1-71 | 51.45 | 16.12 |
| Example 1-72 | not active | 34.66 |
| Example 1-73 | 95.81 | 23.32 |
| Example 1-74 | 72.65 | 29.68 |
| Example 1-75 | not active | 23.51 |
| Example 1-76 | 79.65 | 29.68 |
| Example 1-77 | 50.79 | 27.84 |
| Example 1-78 | 42.24 | 23.32 |
| Example 1-79 | 61.61 | 23.32 |
| Example 1-80 | 56.02 | 16.12 |
| Example 1-81 | 59.84 | 33.19 |
| Example 1-82 | 94.09 | 34.66 |
| Example 1-83 | not active | 23.51 |
| Example 1-84 | 26.01 | 33.87 |
| Example 1-85 | 53.78 | 23.32 |
| Example 1-86 | not active | 23.32 |
| Example 1-87 | 71.67 | 65.2 |
| Example 1-88 | 37.79 | 16.12 |
| Example 1-89 | 83.22 | 23.32 |
| Example 1-90 | 48.64 | 27.84 |
| Example 1-91 | 20.62 | 23.32 |
| Example 1-92 | 56.88 | 33.87 |
| Example 1-93 | 58.09 | 33.87 |
| Example 1-94 | 53.46 | 33.87 |
| Example 1-95 | 52.08 | 33.33 |
| Example 1-96 | 98.38 | 33.33 |
| Example 1-97 | not active | 33.33 |
| Example 1-98 | 76.66 | 33.33 |
| Example 1-99 | not active | 33.33 |
| Example 1-100 | 46.12 | 33.87 |
| Example 1-101 | 57.61 | 33.87 |
| Example 1-102 | 35.11 | 23.32 |
| Example 1-103 | 75.43 | 23.32 |
| Example 1-104 | 66.73 | 23.32 |
| Example 1-105 | 39.17 | 23.32 |
| Example 1-106 | 62.00 | 34.66 |
| Example 1-107 | 88.12 | 23.32 |
| Example 1-108 | 76.29 | 23.32 |
| Example 1-109 | not active | 33.33 |
| Example 1-110 | 28.85 | 33.87 |
| Example 1-111 | 54.41 | 33.87 |
| Example 1-112 | not active | 33.33 |
| Example 1-113 | 42.48 | 33.87 |
| Example 1-114 | 48.24 | 33.33 |
| Example 1-115 | 55.24 | 27.84 |
| Example 1-116 | 52.66 | 27.84 |
| Example 1-117 | 72.65 | 44.29 |
| Example 1-118 | 49.17 | 33.87 |
| Example 1-119 | 27.26 | 33.87 |

### Example 3. Verification of In Vitro PLD Inhibitory Activity of Compounds

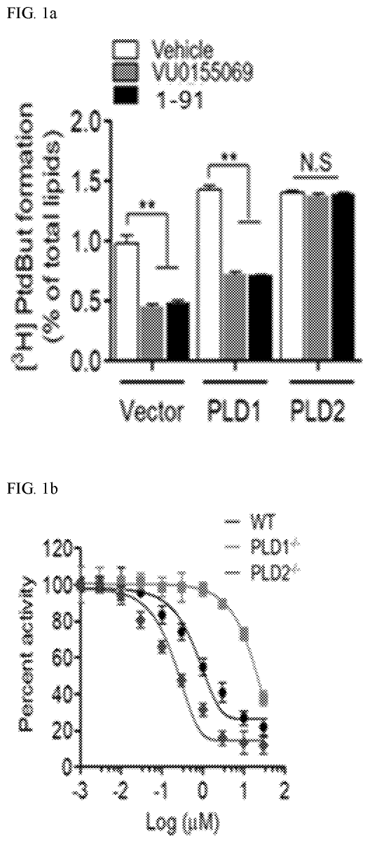

FIG. 1a shows the results of testing the cell activity of PLD using Example 1-91, which exhibited the highest PLD inhibitory activity based on the above results. Figure Ta shows that after overexpressing PLDT and PLD2 in human colon cancer cell line (HCT116) cells, the compound was treated at 2 μM, and PLD activity was measured using the method mentioned in Example 2. It was statistically confirmed that the PLDT inhibitory effect was equal to or greater than that of the positive control VU0155069, and both compounds showed no PLD2 inhibitory effect at a concentration of 2 μM.

FIG. 1b shows the results of measuring PLD activity by treating Example 1-91 compound at different concentrations using mouse embryonic fibroblasts isolated from normal mice, PLD1 knockout mice, and PLD2 knockout mice on the 14th day of embryo development. It was confirmed that the PLD activity decreased first in fibroblasts with PLD2 knockout, indicating that it specifically inhibits PLD1.

### Example 4. Confirmation of Cytotoxicity of Compounds on Cancer Cells

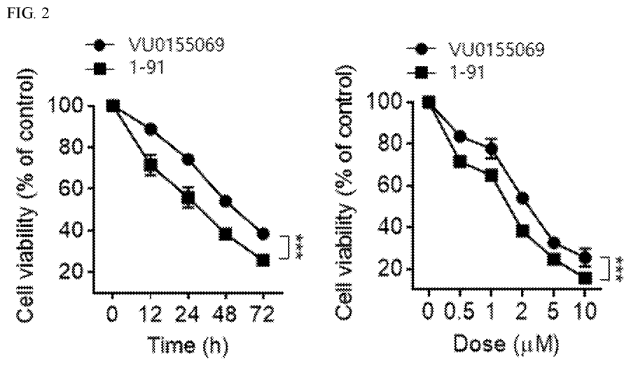

To confirm the cytotoxicity on cancer cells, human colon cancer cell line DLD1 cells were dispensed into a 96-well plate and cultured for 24 hours, then treated with the compound at 2 μM, and cell samples were prepared at 12 hours, 24 hours, 48 hours, and 72 hours. The cytotoxicity of the compound over time was confirmed using the MTT (3-(4,5-dimethylthiazol-2-yl)-2,5-diphenyltetrazolium bromide) method. Additionally, the compound was treated at concentrations of 0.5, 1, 2, 5, and 10 μM in the same cell line, and cytotoxicity was confirmed after 48 hours, showing statistically significant cytotoxicity on cancer cells compared to the positive control VU0155069 (FIG. 2).

### Example 5. Verification of Cell Cycle of Compounds on Normal and Cancer Cells

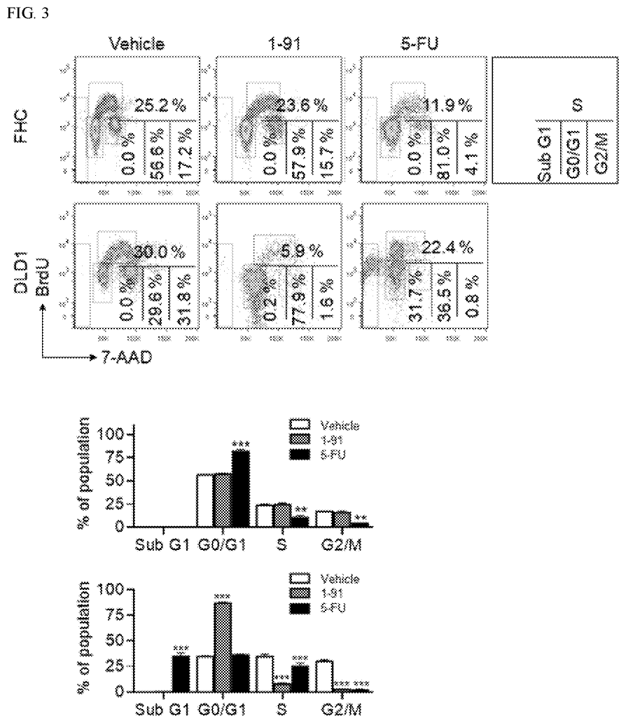

To observe cell cycle changes, normal colon epithelial cells (FHC) and human colon cancer cell line DLD1 were treated with compound 1-91 at 2 μM and the positive control 5-FU at 10 μM. After 48 hours, the cells were washed with PBS, harvested, and sequentially treated with bromodeoxyuridine (BrdU) and 7-aminoactinomycin D (7-AAD). The cells were then stained with an anti-BrdU antibody conjugated with fluorescein isothiocyanate (FITC) and analyzed using a flow cytometer. As shown in FIG. 3, the positive control 5-FU arrested the cell cycle at the G0/G1 phase in normal cells and at the Sub G1 phase in cancer cells, whereas compound 1-91 specifically arrested the cell cycle at the G0/G1 phase only in cancer cells (FIG. 3).

### Example 6. Confirmation of Apoptosis in Normal and Cancer Cells by the Compound

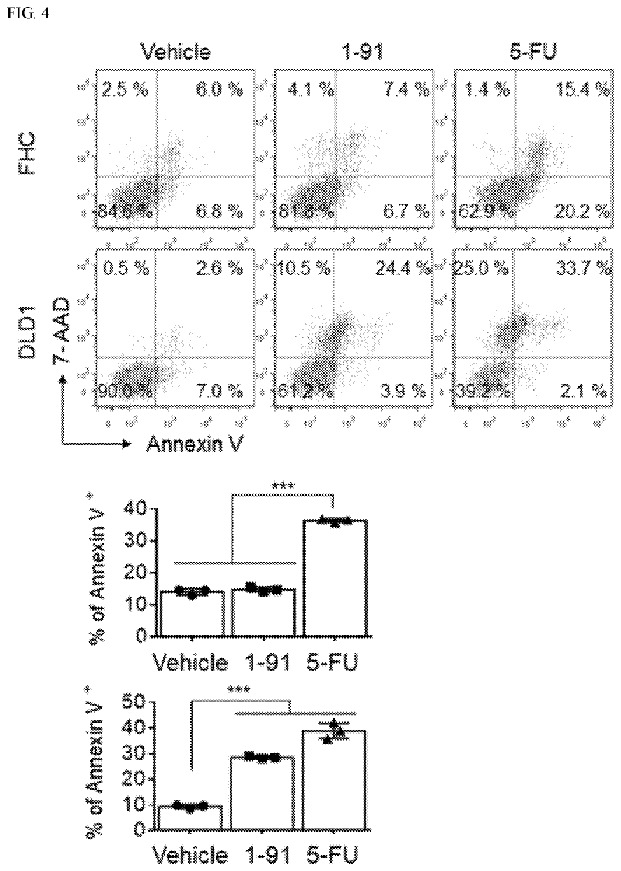

To observe changes in apoptosis, normal colon epithelial cells (FHC) and human colon cancer cell line (DLD1) were treated with compound 1-91 at 2 μM and the positive control 5-FU at 10 μM. After 48 hours, the cells were washed with PBS, harvested, and analyzed for apoptosis using an annexin V-FITC apoptosis detection kit. The positive control 5-FU induced apoptosis in both normal and cancer cells, whereas compound 1-91 specifically and significantly induced apoptosis only in cancer cells (FIG. 4).

### Example 7. Confirmation of Antitumor Effects in an Inflammatory Colon Cancer Animal Model (AOM/DSS Model)

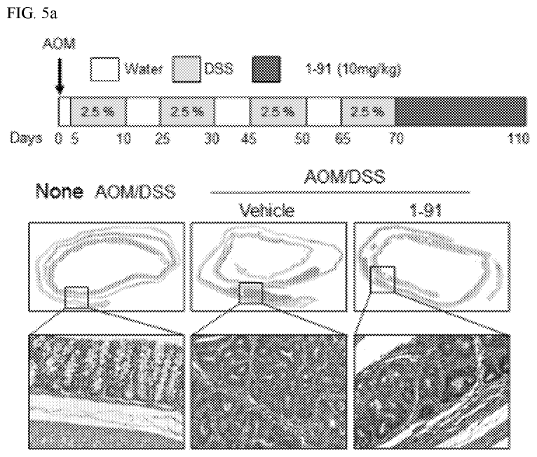

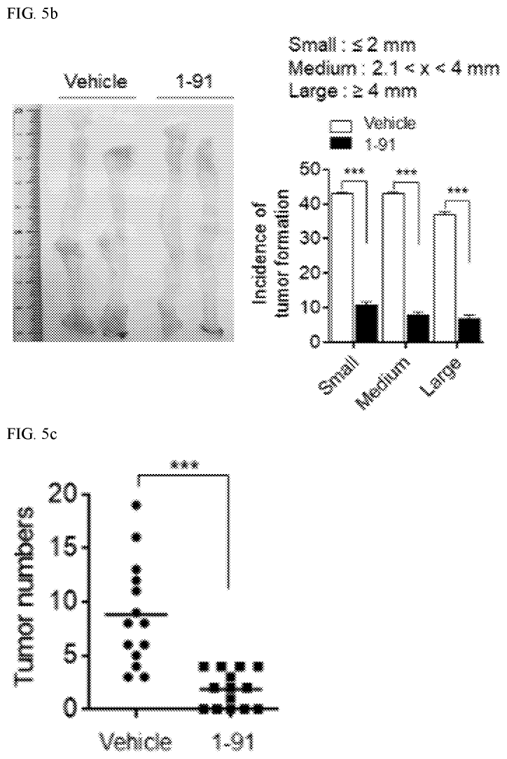

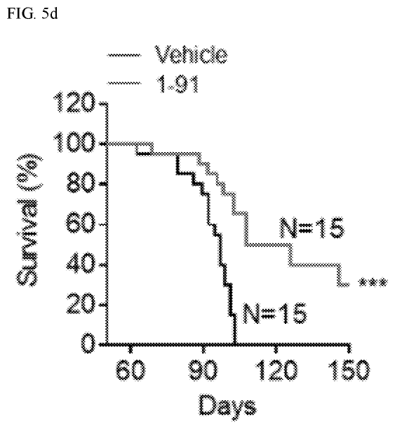

To establish an inflammatory colon cancer animal model, six-week-old C57BL6/N mice were intraperitoneally injected with azoxymethane (AOM) at 10 mg/kg on day 0, followed by administration of 2.5% dextran sulfate sodium (DSS) in drinking water for 5 days and a 15-day recovery period. This DSS cycle was repeated four times. To confirm the effects of the compound in the inflammatory colon cancer animal model, the compound was orally administered at 10 mg/kg every other day for 45 days. After the administration period, the animals were euthanized, and the small intestines were used to prepare paraffin blocks of the intestinal tissue using the Swiss rolling method. The sections were stained with hematoxylin and eosin (H&E). As shown in the lower part of FIG. 5a, cancer tissues were stained in the AOM/DSS model, but almost disappeared in the 1-91 treated group. Additionally, analysis of tumor size and frequency in the small intestines of the animals showed that both the incidence and size of tumors were significantly reduced in the 1-91 treated mice (FIGS. 5b, 5c). Furthermore, survival curve analysis during the experimental period revealed that the survival of the 1-91 treated animals was significantly increased.

### Example 8. Confirmation of Antitumor Effects in an Orthotopic Animal Model

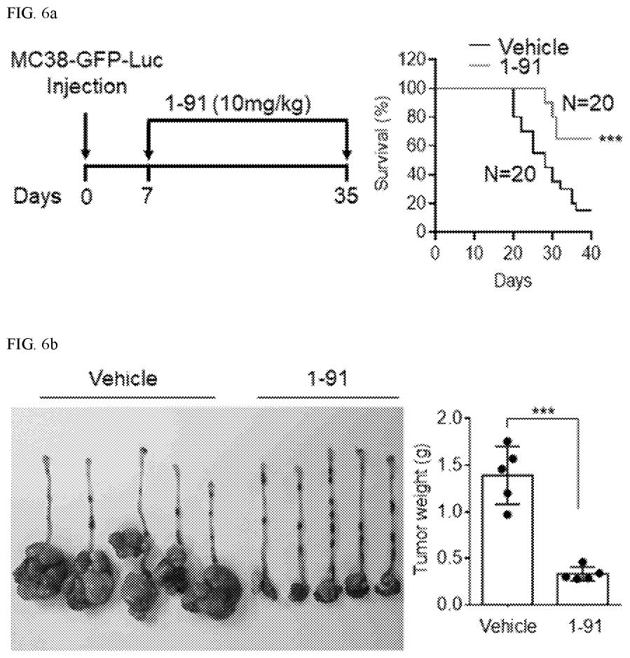

To establish an orthotopic animal model, MC38 mouse colon cancer cells were implanted into the cecum of six-week-old C57BL6/N mice, as shown on the left side of FIG. 6a. For cell implantation, the mice were anesthetized with intraperitoneal injection of 2,2,2-tribromoethanol (avertin), and the cecum was exposed through laparotomy. MC38 cells prepared at 2×10⁵ in 50 μL PBS were implanted. Starting from 7 days after cell implantation, compound 1-91 was orally administered at 10 mg/kg for 2 weeks to confirm the antitumor effects. The right side of FIG. 6a shows the survival curve of the animals during the compound administration period, indicating a significant increase in survival in the 1-91 treated animals. FIG. 6b shows the results of tumor confirmation in the cecum after euthanizing the animals, revealing that the tumor size was significantly reduced by the 1-91 treatment.

### INDUSTRIAL APPLICABILITY

The compound according to the present invention has excellent activity in inhibiting PLD, making it highly useful for the development of treatments for cancer and/or degenerative neurological diseases, so its industrial applicability is very high.

1. A compound represented by the following Chemical Formula 1 or a pharmaceutically acceptable salt thereof: 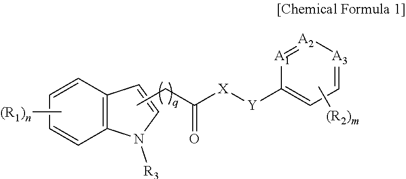
wherein,
R1 is each independently hydrogen, substituted or unsubstituted C1-C10 alkyl, halogen, C1-C10 alkoxycarbonyl, C1-C10 hydroxycarbonyl, or 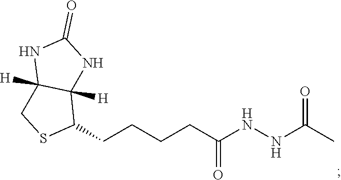
R2 is each independently hydrogen, nitro, halogen, C1-C10 alkoxy, hydroxy, C1-C10 alkoxycarbonyl, substituted or unsubstituted C1-C10 alkylene, or substituted or unsubstituted C1-C6 alkynyl;
R3 is substituted or unsubstituted C1-C10 alkyl;
X is N or 0;
Y is absent, substituted or unsubstituted C1-C10 alkylene or N;
A1, A2 and A3 are each independently C or N;
n is 1, 2 or 3;
m is 1, 2 or 3;
q is 0, 1, 2, 3, 4 or 5; and
when R1, R2, R3 and Y are substituted, they are each independently substituted with one or more substituents selected from the group consisting of halogen, C1-C10 alkyl, nitro, hydroxy, cyano, amino, thiol, carboxyl, amide, nitrile, sulfide, disulfide, sulfonyl, formyl, formyloxy and formylamino.

2. The compound or a pharmaceutically acceptable salt thereof according to claim 1, wherein R1 is each independently hydrogen, halogen, methoxycarbonyl, hydroxycarbonyl, —CF₃ or

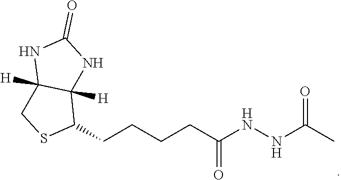

3. The compound or a pharmaceutically acceptable salt thereof according to claim 1, wherein R2 is each independently hydrogen, nitro, halogen, methoxy, hydroxy, methoxycarbonyl, —CF₃, t-butyl, ethoxycarbonyl, ethynyl, 2-propyl, 2-butyl or methyl.

4. The compound or a pharmaceutically acceptable salt thereof according to claim 1, wherein R3 is methyl.

5. The compound or a pharmaceutically acceptable salt thereof according to claim 1, wherein Y is absent, C1-C3 alkylene or N.

6. The compound or a pharmaceutically acceptable salt thereof according to claim 1, wherein one of A1, A2 and A3 is N and the remaining two are C.

7. The compound or a pharmaceutically acceptable salt thereof according to claim 1, wherein the compound of Chemical Formula 1 is selected from the group consisting of the following compounds:
N-(4-chloro-3-nitrophenyl)-1H-indole-3-carboxamide,
N-(4-bromophenyl)-1H-indole-3-carboxamide,
N-(3,4-dimethoxyphenyl)-1H-indole-3-carboxamide,
N-(3,4-dichlorophenyl)-1H-indole-3-carboxamide,
N-(3,4-dichlorobenzyl)-1H-indole-3-carboxamide,
N′-(3,4-dichlorophenyl)-1H-indole-2-carbohydrazide,
N-(2-hydroxyphenyl)-2-(1H-indol-3-yl)acetamide,
N-(3-hydroxyphenyl)-2-(1H-indol-3-yl)acetamide,
2-(1H-indol-3-yl)-N-(2-methoxyphenyl)acetamide,
2-(1H-indol-3-yl)-N-(3-methoxyphenyl)acetamide,
2-(1H-indol-3-yl)-N-(4-methoxyphenyl)acetamide,
N-(2-fluorophenyl)-2-(1H-indol-3-yl)acetamide,
N-(3-fluorophenyl)-2-(1H-indol-3-yl)acetamide,
N-(4-fluorophenyl)-2-(1H-indol-3-yl)acetamide,
N-(2-bromophenyl)-2-(1H-indol-3-yl)acetamide,
N-(3-bromophenyl)-2-(1H-indol-3-yl)acetamide,
N-(4-bromophenyl)-2-(1H-indol-3-yl)acetamide,
N-(3,5-difluorophenyl)-2-(1H-indol-3-yl)acetamide,
N-(4-chloro-3-nitrophenyl)-2-(1H-indol-3-yl)acetamide,
N-(3,4-dichlorophenyl)-2-(1H-indol-3-yl)acetamide,
N-(5-bromopyridin-2-yl)-2-(1H-indol-3-yl)acetamide,
N-(5-fluoropyridin-2-yl)-2-(1H-indol-3-yl)acetamide,
2-(1H-indol-3-yl)-N-phenylacetamide,
N-(4-hydroxyphenyl)-2-(1H-indol-3-yl)acetamide,
methyl 4-(2-(1H-indol-3-yl)acetamido)benzoate,
2-(1H-indol-3-yl)-N-(4-(trifluoromethyl)phenyl)acetamide,
N-(4-tert-butylphenyl)-2-(1H-indol-3-yl)acetamide,
N-(3,4-dimethoxyphenyl)-2-(1H-indol-3-yl)acetamide,
ethyl 4-(2-(1H-indol-3-yl)acetamido)benzoate,
2-(1H-indol-3-yl)-N-(3-nitrophenyl)acetamide,
N-(4-ethynylphenyl)-2-(1H-indol-3-yl)acetamide,
N-(3,5-bis(trifluoromethyl)phenyl)-2-(1H-indol-3-yl)acetamide,
2-(1H-indol-3-yl)-N-(4-isopropylphenyl)acetamide,
N-(4-sec-butylphenyl)-2-(1H-indol-3-yl)acetamide,
N-(2-fluorobenzyl)-2-(1H-indol-3-yl)acetamide,
N-(3-fluorobenzyl)-2-(1H-indol-3-yl)acetamide,
N-(4-fluorobenzyl)-2-(1H-indol-3-yl)acetamide,
methyl 4-((2-(1H-indol-3-yl)acetamido)methyl)benzoate,
2-(1H-indol-3-yl)-N-(4-methoxybenzyl)acetamide,
2-(1H-indol-3-yl)-N-(3-methylbenzyl)acetamide,
N-(4-tert-butylbenzyl)-2-(1H-indol-3-yl)acetamide,
2-(1H-indol-3-yl)-N-(3-(trifluoromethyl)benzyl)acetamide,
2-(1H-indol-3-yl)-N-(4-(trifluoromethyl)benzyl)acetamide,
N-(2,4-dimethoxybenzyl)-2-(1H-indol-3-yl)acetamide,
N-(3,4-dimethoxybenzyl)-2-(1H-indol-3-yl)acetamide,
2-(1H-indol-3-yl)-N-(4-nitrobenzyl)acetamide,
N-(2,3-dichlorobenzyl)-2-(1H-indol-3-yl)acetamide,
N-(2-chlorobenzyl)-2-(1H-indol-3-yl)acetamide,
N-(4-chlorobenzyl)-2-(1H-indol-3-yl)acetamide,
2-(1H-indol-3-yl)-N-(2-methoxybenzyl)acetamide,
N′-(2-chlorophenyl)-2-(1H-indol-3-yl)acetohydrazide,
N′-(3-chlorophenyl)-2-(1H-indol-3-yl)acetohydrazide,
N′-(4-chlorophenyl)-2-(1H-indol-3-yl)acetohydrazide,
N′-(3,4-dichlorophenyl)-2-(1H-indol-3-yl)acetohydrazide,
N′-(4-bromophenyl)-2-(1H-indol-3-yl)acetohydrazide,
2-(1H-indol-3-yl)-N-(pyridin-2-ylmethyl)acetamide,
2-(1H-indol-3-yl)-N-(pyridin-3-ylmethyl)acetamide,
2-(1H-indol-3-yl)-N-(pyridin-4-ylmethyl)acetamide,
2-(1H-indol-3-yl)-N-phenethylacetamide,
N-(4-bromophenethyl)-2-(1H-indol-3-yl)acetamide,
N-(2-fluorophenyl)-3-(1H-indol-3-yl)propanamide,
N-(3-fluorophenyl)-3-(1H-indol-3-yl)propanamide,
N-(4-fluorophenyl)-3-(1H-indol-3-yl)propanamide,
N-(3-bromophenyl)-3-(1H-indol-3-yl)propanamide,
N-(4-bromophenyl)-3-(1H-indol-3-yl)propanamide,
N-(3,5-difluorophenyl)-3-(1H-indol-3-yl)propanamide,
N-(3,5-bis(trifluoromethyl)phenyl)-3-(1H-indol-3-yl)propanamide,
N-(4-chloro-3-nitrophenyl)-3-(1H-indol-3-yl)propanamide,
N-(3,4-dichlorophenyl)-3-(1H-indol-3-yl)propanamide,
N-(3,4-dimethoxyphenyl)-3-(1H-indol-3-yl)propanamide,
3-(1H-indol-3-yl)-N-(4-methoxybenzyl)propanamide,
N-(3,4-dichlorobenzyl)-3-(1H-indol-3-yl)propanamide,
N-(3,4-dimethoxybenzyl)-3-(1H-indol-3-yl)propanamide,
N-(3-fluorophenyl)-4-(1H-indol-3-yl)butanamide,
N-(4-fluorophenyl)-4-(1H-indol-3-yl)butanamide,
N-(4-bromophenyl)-4-(1H-indol-3-yl)butanamide,
N-(3,5-bis(trifluoromethyl)phenyl)-4-(1H-indol-3-yl)butanamide,
N-(4-chloro-3-nitrophenyl)-4-(1H-indol-3-yl)butanamide,
N-(3,4-dichlorophenyl)-4-(1H-indol-3-yl)butanamide,
methyl 4-((4-(1H-indol-3-yl)butanamido)methyl)benzoate,
4-(1H-indol-3-yl)-N-(4-methoxybenzyl)butanamide,
N-(3,4-dichlorobenzyl)-4-(1H-indol-3-yl)butanamide,
N-(4-fluorophenyl)-1H-indole-2-carboxamide,
N-(3,5-bis(trifluoromethyl)phenyl)-1H-indole-2-carboxamide,
N-(4-chloro-3-nitrophenyl)-1H-indole-2-carboxamide,
N-(3,4-dichlorophenyl)-1H-indole-2-carboxamide,
N-(3,4-dimethoxyphenyl)-1H-indole-2-carboxamide,
methyl 4-((1H-indole-2-carboxamido)methyl)benzoate,
N′-(3,4-dichlorophenyl)-1H-indole-2-carbohydrazide,
5-fluoro-N-(4-fluorophenyl)-1H-indole-2-carboxamide,
N-(3,5-bis(trifluoromethyl)phenyl)-5-fluoro-1H-indole-2-carboxamide,
N-(3,5-difluorophenyl)-5-fluoro-1H-indole-2-carboxamide,
N-(3,4-dimethoxyphenyl)-5-fluoro-1H-indole-2-carboxamide,
N-(3,4-dimethoxybenzyl)-5-fluoro-1H-indole-2-carboxamide,
5-fluoro-N-(3-(trifluoromethyl)phenyl)-1H-indole-2-carboxamide,
N-(3,5-dimethoxyphenyl)-5-fluoro-1H-indole-2-carboxamide,
5-fluoro-N-(2-fluorophenyl)-1H-indole-2-carboxamide,
5-fluoro-N-(3-fluorophenyl)-1H-indole-2-carboxamide,
N-(3,5-bis(trifluoromethyl)phenyl)-5-chloro-1H-indole-2-carboxamide,
methyl 2-((3,5-bis(trifluoromethyl)phenyl)carbamoyl)-1H-indole-5-carboxylate,
2-((3,5-bis(trifluoromethyl)phenyl)carbamoyl)-1H-indole-5-carboxylic acid,
N-(3,5-bis(trifluoromethyl)phenyl)-5-(trifluoromethyl)-1H-indole-2-carboxamide,
N-(3,5-dimethylphenyl)-5-fluoro-1H-indole-2-carboxamide,
N-(3,5-dimethylphenyl)-1H-indole-2-carboxamide,
N-(3,4-dihydroxyphenyl)-1H-indole-3-carboxamide,
N-(3,4-dihydroxyphenyl)-2-(1H-indol-3-yl)acetamide,
N-(3,4-dihydroxybenzyl)-2-(1H-indol-3-yl)acetamide,
N-(3,4-dihydroxyphenyl)-3-(1H-indol-3-yl)propanamide,
N-(3,4-dihydroxyphenyl)-1H-indole-2-carboxamide,
N-(3,4-dihydroxyphenyl)-5-fluoro-1H-indole-2-carboxamide,
N-(3,4-dihydroxybenzyl)-5-fluoro-1H-indole-2-carboxamide,
N-(3,5-dihydroxyphenyl)-5-fluoro-1H-indole-2-carboxamide,
N-(3,5-bis(trifluoromethyl)phenyl)-5,6-difluoro-1H-indole-2-carboxamide,
N-(3,5-bis(trifluoromethyl)phenyl)-5,6-difluoro-1-methyl-1H-indole-2-carboxamide,
4-nitrobenzyl 2-(1H-indol-3-yl)acetate,
4-methoxybenzyl 1H-indole-2-carboxylate,
4-methoxybenzyl 4-(1H-indol-3-yl)butanoate,
3,5-bis(trifluoromethyl)phenyl 5-fluoro-1H-indole-2-carboxylate or
N-(3,5-bis(trifluoromethyl)phenyl)-5-(2-(5-((3aS,4S,6aR)-2-oxohexahydro-1H-thieno[3,4-d]imidazol-4-yl)pentanoyl)hydrazine-1-carbonyl)-1H-indole-2-carboxamide.

8. A pharmaceutical composition for preventing or treating cancer or degenerative neurological diseases, comprising a compound of claim 1 or a pharmaceutically acceptable salt thereof as an active ingredient.

9. The pharmaceutical composition according to claim 8, wherein the cancer is selected from the group consisting of blood cancer, multiple myeloma, acute myeloid leukemia, malignant lymphoma, aplastic anemia, thymic cancer, ovarian cancer, cervical cancer, breast cancer, colorectal cancer, liver cancer, stomach cancer, pancreatic cancer, colon cancer, peritoneal metastatic cancer, skin cancer, bladder cancer, prostate cancer, thyroid cancer, lung cancer, osteosarcoma, fibrous tumor, and brain tumor.

10. The pharmaceutical composition according to claim 8, wherein the neurodegenerative disease is selected from the group consisting of cognitive dysfunction, dementia, Alzheimer's disease, Parkinson's disease, Lou Gehrig's disease, Huntington's disease, amyotrophic lateral sclerosis, multiple sclerosis, immune system abnormality brain dysfunction, progressive neurodegenerative disease, metabolic encephalopathy, Niemann-Pick disease, Pick's disease, dementia due to cerebral ischemia, and dementia due to cerebral hemorrhage.

11. Use of a compound of claim 1 or a pharmaceutically acceptable salt thereof for preparing a pharmaceutical composition for treating cancer or a degenerative neurological disease.

12. A method for treating cancer or a degenerative neurological disease comprising administering an effective amount of a pharmaceutical composition comprising a compound of claim 1 or a pharmaceutically acceptable salt thereof as an active ingredient to a subject in need thereof.
